(19) United States
(12) Patent Application Publication
(10) Pub. No.: US 2021/0398682 A1
(43) Pub. Date: Dec. 23, 2021

(54) Methods for Evaluation of Gestational Progress and Preterm Abortion for Clinical Intervention and Applications Thereof

(71) Applicants: The Board of Trustees of the Leland Stanford Junior University, Stanford, CA (US); Statens Serum Institut, Copenhagen S (DK)

(72) Inventors: Liang Liang, Palo Alto, CA (US); Jijuan Gu Urban, Palo Alto, CA (US); Mads Melbye, Copenhagen S (DK); Michael P. Snyder, Stanford, CA (US)

(73) Assignees: The Board of Trustees of the Leland Stanford Junior University, Stanford, CA (US); Statens Serum Institut, Copenhagen S (DK)

(21) Appl. No.: 17/207,541

(22) Filed: Mar. 19, 2021

Related U.S. Application Data
(63) Continuation of application No. PCT/US2019/052515, filed on Sep. 23, 2019.
(60) Provisional application No. 62/734,725, filed on Sep. 21, 2018.

(51) Int. Cl. G16H 50/30; G16H 50/20; G16H 10/60

(52) U.S. Cl. CPC G16H 50/30; G16H 10/60; G16H 50/20

(57) ABSTRACT

Methods to compute gestational age and gestational health and applications thereof are described. Generally, systems utilize analyte measurements to determine a gestational age and gestational health, which can be used as a basis to perform interventions and treat individuals.

### CROSS REFERENCE TO RELATED APPLICATIONS

This application is a continuation of PCT Patent Application No. PCT/US2019/052515, filed Sep. 23, 2019, entitled “Methods for Evaluation of Gestational Progress and Preterm Abortion for Clinical Intervention and Applications Thereof” to Liang et al., which claims priority to U.S. Provisional Application Ser. No. 62/734,725, entitled “METHODS FOR ESTIMATING GESTATIONAL AGE, TIME TO DELIVERY AND LABOR ONSET USING METABOLOMIC PROFILING DURING PREGNANCY” to Liang et al., filed Sep. 21, 2018, which are incorporated herein by reference in their entireties.

### FIELD OF THE INVENTION

The invention is generally directed to processes to evaluate gestational progress and applications thereof, and more specifically to methods for evaluating gestational age, time to labor, preterm birth, and preterm abortion including diagnostics to be utilized for clinical interventions.

### BACKGROUND

Pregnancy is one of the most critical periods for mother and child. It involves a tremendous flow of physiological changes and metabolic adaptations week by week, and even small deviations from the norm may have detrimental consequences. There are 300,000 pregnancy and birth-related maternal deaths and 7.5 million perinatal deaths annually worldwide. In addition, 30% of all pregnancies end in miscarriage (<20 weeks), and preterm birth (<37 weeks). The latter is the leading cause of global neonatal morbidity and mortality and is observed for 7-17% of all pregnancies. With 170 million pregnancies yearly worldwide, even small improvements in obstetric health care, based on a better understanding of how pregnancy is regulated, may impact on the wellbeing of a large number of women and children.

Although ultrasound is used in clinics for estimating the gestational age, its accuracy is suboptimal with only 40% of the newborns delivered within 7 days of the predicted due dates. The accuracy is also decreased after the first trimester. Thus, there remains a need in the art for improved methods of estimating gestational age and predicting time to delivery and labor onset.

### SUMMARY OF THE INVENTION

In an embodiment for treating a suspected pregnant individual, panel of analytes derived from a sample obtained from an individual is measured. Gestational age of the individual is determined. The individual treated based on the gestational age. The treatment is one of: medication, dietary supplement, Caesarian delivery, or surgical procedure.

In another embodiment, the gestational age of the individual is determined by a computational model.

In yet another embodiment, the computational model is one of: ridge regression, K-nearest neighbors, LASSO regression, elastic net, least angle regression (LAR), random forest, or principal components analysis.

In a further embodiment, a feature in the model is a measurement of at least one of the following metabolites: N,N′-Dicarbobenzyloxy-L-omithine, 1-(1Z-Hexadecenyl)-sn-glycero-3-phosphoethanolamine (PE(P-16:0e/0:0)), delta4-Dafachronic acid, C29H3609, 7alpha,24-Dihydroxy-4-cholesten-3-one, C22H43O12P, C27H44O9, C19H28O7S, Androstane-3,17-diol, 21-Hydroxypregnenolone, Estriol-16-Glucuronide, C25H40O9, C27H44O4, C27H42O3, bilobol, [1-(3,5-dihydroxyphenyl)-12-hydroxytridecan-2-yl] acetate, C26H52NO8P, C27H42O8, Prolylphenylalanine, N,N,Diacetyl-Lys-DAla-DAla, C23H49N2O5P, C21H29O, C33H53O9, C22H35O3, C30H44NO3S, 1,1′-(1,8-dioxo-1,8-octanediyl)bis[glycyl-glycine], C27H42010, 6-ketoestriol sulfate, DAH-3-Keto-4-en, progesterone (m/z: 315, RT/min: 9.3), progesterone (m/z 337, RT/min 9.3), metabolite (m/z: 511, RT/min: 5.4), metabolite (m/z: 519, RT/min: 8.6), metabolite (m/z: 563, RT/min: 6.6), metabolite (m/z: 353, RT/min: 7.9), metabolite (m/z: 487, RT/min: 6.6), metabolite (m/z: 319, RT/min: 2.6), metabolite (m/z: 821, RT/min: 9.1), metabolite (m/z: 653, RT/min: 9.3), metabolite (m/z: 798, RT/min: 8.5), metabolite (m/z: 260, RT/min: 9.8), and metabolite (m/z: 823, RT/min: 9.3).

In still yet another embodiment, a feature in the model is a measurement of at least one of the following protein constituents: NTRK2, LAIR2, CD200R1, LXN, DRAXIN, ROBO2, CD93, NTRK3, MDGA1, CRTAM, IL12B/IL12A, RGMA, IL2RA, ESM1, FcRL2, UPAR, MCP2, IL5Ralpha, CLM1, uPA, CCL28, PCSK9, PDGFRalpha, SMPD1, SKR3, DLK1, NRP2, MSR1, GMCSFRalpha, CTSC, RET, SMOC2, PRTG, PVRL4, ST2, NrCAM, SYND1, TNFRSF12A, DDR1, CD200, GRN, or PAI1.

In yet a further embodiment, a feature in the model is a measurement of at least one of the following metabolites: THDOC, estriol-16-glucoronide, progesterone, PE(P-16:0e/0:0), or DHEA-S.

In an even further embodiment, the model predicts gestational age of 20 weeks.

A feature in the model is a measurement of at least one of the following metabolites: estriol-16-glucoronide or progesterone.

In yet an even further embodiment, the model predicts gestational age of 24 weeks. A feature in the model is a measurement of at least one of the following metabolites: THDOC, estriol-16-glucoronide, or progesterone.

In still yet an even further embodiment, the model predicts gestational age of 28 weeks. A feature in the model is a measurement of at least one of the following metabolites: THDOC or progesterone.

In still yet an even further embodiment, the model predicts gestational age of 32 weeks. A feature in the model is a measurement of at least one of the following metabolites: THDOC or estriol-16-glucoronide.

In still yet an even further embodiment, the model predicts gestational age of 37 weeks. A feature in the model is a measurement of at least one of the following metabolites: THDOC, estriol-16-glucoronide, or androstane-3,17-diol.

In still yet an even further embodiment, the model predicts 8 weeks to delivery. A feature in the model is a measurement of at least one of the following metabolites: THDOC or alpha-hydroxyprogesterone.

In still yet an even further embodiment, the model predicts 4 weeks to delivery. A feature in the model is a measurement of at least one of the following metabolites: THDOC, estriol-16-glucoronide, or PE(P-16:0e/0:0).

In still yet an even further embodiment, the model predicts 2 weeks to delivery. A feature in the model is a measurement of at least one of the following metabolites: THDOC, estriol-16-glucoronide, or androstane-3,17-diol.

In still yet an even further embodiment, the model utilizes a plurality of analyte measurement features. The analyte measurement features are determined by their contribution to the predictive power of the model.

In still yet an even further embodiment, the sample is one of: a blood sample, a stool sample, a urine sample, a saliva sample, or a biopsy of the individual.

In still yet an even further embodiment, the analytes are extracted and measured with periodicity.

In still yet an even further embodiment, the individual has been diagnosed as pregnant.

In still yet an even further embodiment, the individual has not been diagnosed as pregnant.

In still yet an even further embodiment, sonography is performed on the individual.

In an embodiment for performing a clinical assessment on a suspected pregnant individual, a panel of analytes derived from a sample obtained from an individual is measured. The gestational age of the individual is determined.

In another embodiment, A clinical assessment on the individual is performed based on the gestational age. The clinical assessment is one of: medical imaging, periodic medical checkups, fetal monitoring, blood tests, microbial culture tests, genetic screening, chorionic villus sampling, or amniocentesis.

In yet another embodiment, the gestational age of the individual is determined by a computational model.

In a further embodiment, the computational model is one of: ridge regression, K-nearest neighbors, LASSO regression, elastic net, least angle regression (LAR), random forest, or principal components analysis.

In still yet another embodiment, a feature in the model is a measurement of at least one of the following metabolites: N,N′-Dicarbobenzyloxy-L-omithine, 1-(1Z-Hexadecenyl)-sn-glycero-3-phosphoethanolamine (PE(P-16:0e/0:0)), delta4-Dafachronic acid, C29H3609, 7alpha,24-Dihydroxy-4-cholesten-3-one, C22H43012P, C27H4409, C19H2807S, Androstane-3,17-diol, 21-Hydroxypregnenolone, Estriol-16-Glucuronide, C25H4009, C27H4404, C27H4203, bilobol, [1-(3,5-dihydroxyphenyl)-12-hydroxytridecan-2-yl] acetate, C26H52NO8P, C27H4208, Prolylphenylalanine, N,N,Diacetyl-Lys-DAla-DAla, C23H49N205P, C21H290, C33H5309, C22H3503, C30H44NO3S, 1,1′-(1,8-dioxo-1,8-octanediyl)bis[glycyl-glycine], C27H42010, 6-ketoestriol sulfate, DAH-3-Keto-4-en, progesterone (m/z: 315, RT/min: 9.3), progesterone (m/z 337, RT/min 9.3), metabolite (m/z: 511, RT/min: 5.4), metabolite (m/z: 519, RT/min: 8.6), metabolite (m/z: 563, RT/min: 6.6), metabolite (m/z: 353, RT/min: 7.9), metabolite (m/z: 487, RT/min: 6.6), metabolite (m/z: 319, RT/min: 2.6), metabolite (m/z: 821, RT/min: 9.1), metabolite (m/z: 653, RT/min: 9.3), metabolite (m/z: 798, RT/min: 8.5), metabolite (m/z: 260, RT/min: 9.8), and metabolite (m/z: 823, RT/min: 9.3).

In yet a further embodiment, a feature in the model is a measurement of at least one of the following protein constituents: NTRK2, LAIR2, CD200R1, LXN, DRAXIN, ROBO2, CD93, NTRK3, MDGA1, CRTAM, IL12B/IL12A, RGMA, IL2RA, ESM1, FcRL2, UPAR, MCP2, IL5Ralpha, CLM1, uPA, CCL28, PCSK9, PDGFRalpha, SMPD1, SKR3, DLK1, NRP2, MSR1, GMCSFRalpha, CTSC, RET, SMOC2, PRTG, PVRL4, ST2, NrCAM, SYND1, TNFRSF12A, DDR1, CD200, GRN, or PAI1.

In an even further embodiment, a feature in the model is a measurement of at least one of the following metabolites: THDOC, estriol-16-glucoronide, progesterone, PE(P-16:0e/0:0), or DHEA-S.

In yet an even further embodiment, the model predicts gestational age of 20 weeks. A feature in the model is a measurement of at least one of the following metabolites: estriol-16-glucoronide or progesterone.

In still yet an even further embodiment, the model predicts gestational age of 24 weeks. A feature in the model is a measurement of at least one of the following metabolites: THDOC, estriol-16-glucoronide, or progesterone.

In still yet an even further embodiment, the model predicts gestational age of 28 weeks. A feature in the model is a measurement of at least one of the following metabolites: THDOC or progesterone.

In still yet an even further embodiment, the model predicts gestational age of 32 weeks. A feature in the model is a measurement of at least one of the following metabolites: THDOC or estriol-16-glucoronide.

In still yet an even further embodiment, the model predicts gestational age of 37 weeks. A feature in the model is a measurement of at least one of the following metabolites: THDOC, estriol-16-glucoronide, or androstane-3,17-diol.

In still yet an even further embodiment, the model predicts 8 weeks to delivery. A feature in the model is a measurement of at least one of the following metabolites: THDOC or alpha-hydroxyprogesterone.

In still yet an even further embodiment, the model predicts 4 weeks to delivery. A feature in the model is a measurement of at least one of the following metabolites: THDOC, estriol-16-glucoronide, or PE(P-16:0e/0:0).

In still yet an even further embodiment, the model predicts 2 weeks to delivery. A feature in the model is a measurement of at least one of the following metabolites: THDOC, estriol-16-glucoronide, or androstane-3,17-diol.

In still yet an even further embodiment, the model utilizes a plurality of analyte measurement features. The analyte measurement features are determined by their contribution to the predictive power of the model.

In still yet an even further embodiment, the sample is one of: a blood sample, a stool sample, a urine sample, a saliva sample, or a biopsy of the individual.

In still yet an even further embodiment, the analytes are extracted and measured with periodicity.

In still yet an even further embodiment, the individual has not been diagnosed as pregnant.

In still yet an even further embodiment, sonography is performed on the individual.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 provides a process for performing diagnostics and/or treating a pregnant individual based on their analyte data in accordance with an embodiment.

FIG. 2 provides a process to construct and train a computational model to determine a pregnant individual's gestational progress and/or gestational health in accordance with an embodiment.

FIG. 3 provides a process to perform a diagnostic and/or treat a pregnant individual based on the individual's computed indication of gestational progress and/or gestational health in accordance with an embodiment.

FIG. 4 provides data of prediction power of five analyte measurement features, utilized in accordance with various embodiments.

FIG. 5 provides various analyte measurement features that are predictive of a number of gestational time points, utilized in accordance with various embodiments of the invention.

FIG. 6 provides data of ElasticNet score of twenty protein constituent measurement features, utilized in accordance with various embodiments of the invention.

FIGS. 7 and 8 each provide a schematic of an experimental design to measure analytes of pregnant women, utilized in accordance with various embodiments.

FIGS. 9 to 11 each provide clustering data of metabolites measured of pregnant women, utilized in accordance with various embodiments.

FIG. 12 provides the top metabolites that increased during gestation, utilized in accordance with various embodiments.

FIG. 13 provides the top metabolites that decreased during gestation, utilized in accordance with various embodiments.

FIG. 14 provides a correlation matrix colored by the Pearson correlation coefficient of each pair of pregnancy-related compounds identified by in-house library across samples, utilized in accordance with various embodiments.

FIGS. 15 to 17 provides data graphs depicting average levels of the metabolite changes against the gestational progression for various metabolite groups, utilized in accordance with various embodiments.

FIG. 18 provides a KEGG pathway analysis of metabolites identified, utilized in accordance with various embodiments.

FIG. 19 provides a heatmap showing the temporal changes of pregnancy-related pathway activities during pregnancy and postpartum (PP), utilized in accordance with various embodiments.

FIG. 20 provides a depiction of the steroid hormone biosynthesis pathway, utilized in accordance with various embodiments.

FIG. 21 provides data on organs that produce metabolites, utilized in accordance with various embodiments.

FIG. 22 provides a depiction of the arachidonic acid metabolism pathway, utilized in accordance with various embodiments.

FIG. 23 provides data on medical conditions that correlated with pregnancy-related metabolites, utilized in accordance with various embodiments.

FIG. 24 provides gestational age (GA) predicted by five identified metabolites (y-axis) and its concordance to clinical values determined by standard of care (first-trimester ultrasound, x-axis), generated in accordance with various embodiments.

FIG. 25 provides results of metabolic measurement selection for GA prediction, utilized in accordance with various embodiments.

FIG. 26 provides gestational age (GA) predicted by five identified metabolites (y-axis) and its concordance to clinical values determined by standard of care (first-trimester ultrasound, x-axis), generated in accordance with various embodiments.

FIG. 27 provides data on correlated patterns of the predicted GA with the actual GA at the individual level in the cross validation, generated in accordance with various embodiments.

FIG. 28 provides a comparison of the accuracy of metabolite-predicted delivery (in red) to published general ultrasound accuracy, generated in accordance with various embodiments.

FIG. 29 provides results of feature selection for GA prediction using identified metabolites, utilized in accordance with various embodiments.

FIG. 30 provides data on correlated patterns of the predicted GA with the actual GA at the individual level in the cross validation, generated in accordance with various embodiments.

FIG. 31 provides data showing gestational age (GA) predicted by the five metabolites (y-axis) is highly concordant to clinical values determined by standard of care (first-trimester ultrasound, x-axis) in the validation-2 cohort, generated in accordance with various embodiments.

FIGS. 32, 33, and 34 provide measured MS/MS fragmentation profiles of the five highly predictive metabolites, utilized in accordance with various embodiments.

FIG. 35 provides data on a logistic regression model based on 3 metabolites can accurately distinguish the third trimester plasma samples before or after 37 weeks, generated in accordance with various embodiments.

FIG. 36 provides data on intensity range separations of THDOC and androstane-3,17-diol before/after the 37th week, utilized in accordance with various embodiments.

FIGS. 37 and 38 provide prediction results of models to predict gestational age of 20-weeks, 24-weeks, 28-weeks, and 32-weeks, generated in accordance with various embodiments.

FIG. 39 provides data on a logistic regression model based on 3 metabolites can accurately distinguish the third trimester plasma samples 2 weeks to delivery, generated in accordance with various embodiments.

FIG. 40 provides data on intensity range separations of androstane-3,17-diol and estriol-16-Glucuronide before/after 2-weeks to delivery, generated in accordance with various embodiments.

FIGS. 41 and 42 provide prediction results of models to predict 4-weeks to delivery and 8-weeks to delivery, generated in accordance with various embodiments.

FIGS. 43 and 44 provide measured MS/MS fragmentation profiles matching of androstane-3,17-diol and 17alpha-hydroxyprogesterone, utilized in accordance with various embodiments.

FIG. 45 provides a schematic diagram of targeted plasma proteomic profiling across pregnancy and postpartum time points, utilized in accordance with various embodiments.

FIG. 46 provides gene ontology analysis for various modules identified, utilized in accordance with various embodiments.

FIGS. 47 and 48 each provide data on the reproducibility of detecting protein targets in plasma samples using multiplex PEA, generated in accordance with various embodiments.

FIG. 49 provides performance results of an ElasticNet module, generated in accordance with various embodiments.

FIG. 50 provides fuzzy c-means clustering data for a number of proteins across all gestational months and the postpartum time point, utilized in accordance with various embodiments.

FIG. 51 provides data on the predictability of 40 protein constituents utilized in a model, generated in accordance with various embodiments.

FIG. 52 provides a heatmap showing the changes of levels of all proteins before and after labor using unsupervised hierarchical clustering, utilized in accordance with various embodiments.

FIG. 53 provides data on two distinct clusters that were plotted to show the separation of samples prior to (green triangle) and post (red dot) labor, utilized in accordance with various embodiments.

FIG. 54 provides data on two distinct clusters that were plotted to show the separation of samples, utilized in accordance with various embodiments.

FIG. 55 provides data correlation between protein constituents identified and chromosomal location, utilized in accordance with various embodiments.

FIG. 56 provides data on the levels of 20 proteins that differed significantly between spontaneous abortions (red box, cases) in the first trimester and normal pregnancies (blue box, controls) in the first trimester, utilized in accordance with various embodiments.

FIG. 57 provides measurements of a number of protein constituents over time, utilized in accordance with various embodiments.

FIGS. 58 and 59 provide expression levels of proteins, comparing abortive, normal, and prior to birth, utilized in accordance with various embodiments.

FIG. 60 provides data showing gestational age predicted by a combination of 4 metabolites and 4 protein constituents (y-axis) is highly concordant to clinical values determined by standard of care (first-trimester ultrasound, x-axis) in the validation-2 cohort, generated in accordance with various embodiments.

FIG. 61 provides data of prediction power of eight analyte measurement features (four metabolites and four protein constituents), utilized in accordance with various embodiments.

### DETAILED DESCRIPTION

Turning now to the drawings and data, methods to determine gestational progress and/or gestational health based on analyte measurements derived from a pregnant individual and applications thereof in accordance with various embodiments are described. In some embodiments, a panel of analyte measurements are used to compute gestational progress (i.e., gestational age and/or time to delivery) and provide an indication of an individual's pregnancy timeline. In some embodiments, a panel of analyte measurements are used to compute an indication of a pregnancy health including various complications, such as spontaneous abortion. Many embodiments utilize an individual's gestational age and/or health determination to perform further diagnostic testing and/or treat the individual. In some instances, a diagnostic can include medical imaging (e.g., ultrasonography), periodic medical checkups, fetal monitoring, blood tests (e.g., glucose), microbial culture tests, genetic screening, chorionic villus sampling, and amniocentesis. In some instances, a treatment can include a medication, a dietary supplement, Caesarian delivery, a surgical procedure, and any combination thereof.

Many treatment regimens and clinical decisions in obstetrics depend on an accurate estimation of the timing and progression of pregnancy. Current clinical determination of gestational age and due date are typically based on information about last menstruation date or ultrasound imaging, which can be imprecise. An accurate and cost-effective method for estimating gestational age and delivery time is in need.

The present disclosure is based on the discovery of analyte biomarkers that can be used in monitoring women during pregnancy to determine gestational age, time until delivery, indicate preterm labor, and diagnose spontaneous abortion. Untargeted analyte investigations were performed on weekly blood samples from a cohort of pregnant women (see Exemplary Embodiments). This study revealed analyte alterations during normal pregnancy. Many analyte measurements and the dynamics of the various analytes were shown to be timed precisely according to pregnancy progression and can be used to assess gestational progress, preterm labor and spontaneous abortion. In various embodiments, computational models utilize analyte measurements to determine gestational progress and health.

### Analytes Indicative of Gestational Progress and Health

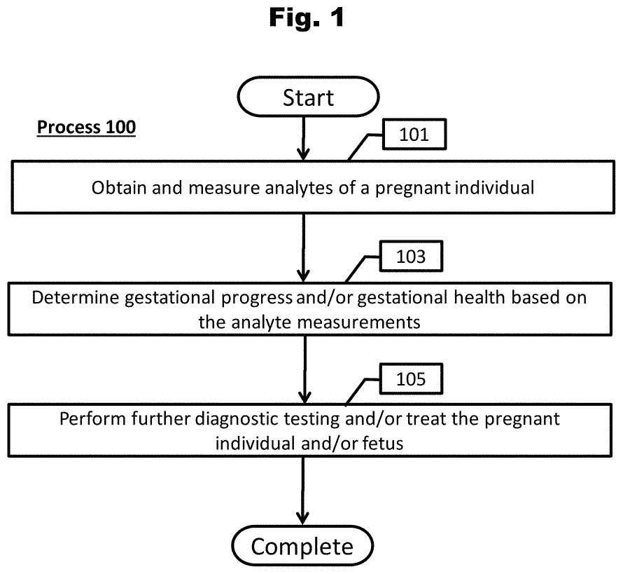

A process for determining pregnancy progress, gestational age, time to delivery, and/or a gestational health using analyte measurements, in accordance with an embodiment of the invention is shown in FIG. 1. This embodiment is directed to determining an indication of gestational progress and/or health of an individual and applies the knowledge garnered to perform further diagnostics and/or treat an individual. For example, this process can be used to identify an individual having a particular analyte constituency that is indicative of spontaneous abortion and treat that individual with estrogen and/or progesterone and further monitor the individual (e.g., weekly medical checkups).

In a number of embodiments, analytes and analyte measurements are to be interpreted broadly as clinical and molecular constituents and measurements that can be captured in medical and/or laboratory setting and are to include metabolites, protein constituents, genomic DNA, transcript expression, and lipids. In some embodiments, metabolites are to include intermediates and products of metabolism such as (for example) sugars, amino acids, nucleotides, antioxidants, organic acids, polyols, vitamins, and the like. In various embodiments, protein constituents are chains of amino acids which are to include (but not limited to) peptides, enzymes, receptors, ligands, antibodies, transcription factors, cytokines, hormones, growth factors and the like. In some embodiments, genomic DNA is DNA of an individual and includes (but is not limited to) copy number variant data, single nucleotide variant data, polymorphism data, mutation analysis, insertions, deletions, epigenetic data and partial and full genomes. In various embodiments, transcript expression is the evidence of RNA molecules of a particular gene or other RNA transcripts, and is to include (but is not limited to) analysis of expression levels of particular transcript targets, splicing variants, a class or pathway of gene targets, and partial and full transcriptomes. In some embodiments, lipids are a broad class of molecules that include (but are not limited to) fatty acid molecules, fat soluble vitamins, glycerolipids, phospholipids, sterols, sphingolipids, prenols, saccharolipids, polyketides, and the like.

In some embodiments, clinical data and/or personal data can be additionally used to indicate gestation age and/or health. In some embodiments, clinical data is to include medical patient data such as (for example) weight, height, heart rate, blood pressure, body mass index (BMI), clinical tests and the like. In various embodiments, personal data is to include data captured by an individual such as (for example) wearable data, physical activity, diet, substance abuse and the like.

Referring back to FIG. 1, process 100 begins with obtaining and measuring (101) analytes from a pregnant individual. In many instances, analytes are measured from a blood extraction, stool sample, urine sample, saliva or biopsy. In some embodiments, an individual's sample is extracted during fasting, or in a controlled clinical assessment. A number of methods are known to extract samples from an individual and can be used within various embodiments of the invention. In several embodiments, analytes are extracted over a period a time (e.g., across pregnancy timeline) and measured at each time point, resulting in a dynamic analysis of the analytes. In some of these embodiments, analytes are measured with periodicity (e.g., weekly, monthly, trimester).

In a number of embodiments, an individual is any individual that has their analytes extracted and measured, especially individuals that have an indication of pregnancy. In some embodiments, an individual has been diagnosed as being pregnant (e.g., as determined by urine test or ultrasound). Embodiments are also directed to an individual being one that has not yet been diagnosed as pregnant.

A number of analytes can be used to indicate gestation age and/or health, including (but not limited to) metabolites, protein constituents, genomic DNA, transcript expression, and lipids. In some embodiments, clinical data and/or personal data can be additionally used to indicate gestation age and/or health. Analytes can be detected and measured by a number of methods, including nucleic acid and protein sequencing, mass spectrometry, colorimetric analysis, immunodetection, and the like.

In several embodiments, analyte measurements are performed by taking a single time-point measurement. In many embodiments, the median and/or average of a number time points for participants with multiple time-point measurements are utilized. Various embodiments incorporate correlations, which can be calculated by a number of methods, such as the Spearman correlation method. A number of embodiments utilize a computational model that incorporates analyte measurements, such as linear regression and elastic net models. Significance can be determined by calculating p-values and/or contribution, which may be corrected for multiple hypotheses testing. It should be noted however, that there are several correlation, computational models, and statistical methods that can utilize analyte measurements and may also fall within some embodiments of the invention.

In a number of embodiments, dynamic correlations use a ratio of analyte measurements between two time points, a percent change of analyte measurements over a period of time, a rate of change of analyte measurements over a period of time, or any combination thereof. Several other dynamic measurements may also be used in the alternative or in combination in accordance with multiple embodiments.

Using static and/or dynamic measures of analytes, process 100 determines (103) gestational progress and/or gestational health based on the analyte measurements. In many embodiments, the correlations and/or computational models can be used to indicate gestational progress and/or gestational health. In several embodiments, determining analyte correlations or modeling gestational progress and/or gestational health is used to substitute other gestational tests, such as (for example) ultrasonography. In various embodiments, measurements of analytes can be used as a precursor indicator to determine whether to perform a further clinical test, such as (for example) ultrasonography.

Having determined an individual's gestational progress and/or gestational health, further diagnostic test can be performed or the pregnant individual and/or fetus can be treated (105). In some instances, a diagnostic can include medical imaging (e.g., ultrasonography), periodic medical checkups, fetal monitoring, blood tests (e.g., glucose), microbial culture tests, genetic screening, chorionic villus sampling, amniocentesis, and any combination thereof. In some instances, a treatment can include a medication, a dietary supplement, Caesarian delivery, a surgical procedure, and any combination thereof.

While specific examples of determining an individual's gestational progress and/or gestational health are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for determining an individual's gestational progress and/or gestational health appropriate to the requirements of a given application can be utilized in accordance with various embodiments of the invention.

Modeling Gestational Progress and Health with Analyte Measurements

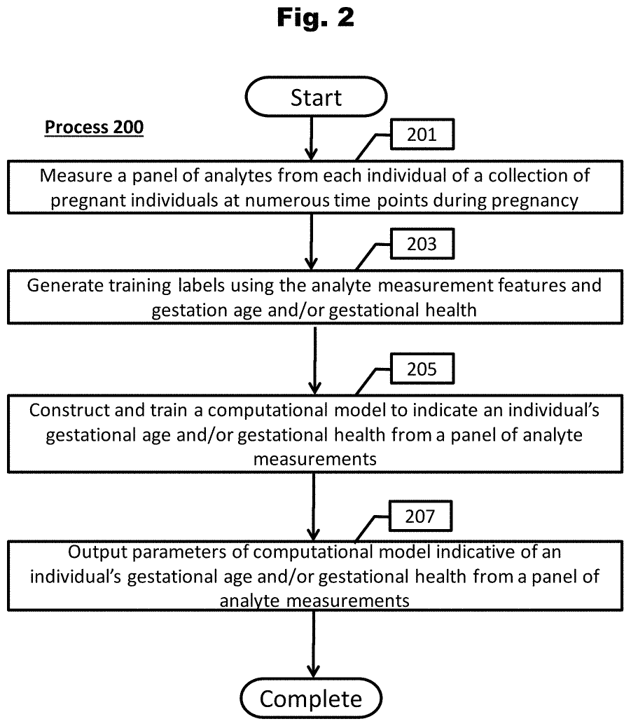

A process for constructing and training a computational model to indicate gestational progress and/or gestational health in accordance with an embodiment of the invention is shown in FIG. 2. Process 200 measures (201) a panel of analytes from each individual of a collection of pregnant individuals numerous times during pregnancy. In several embodiments, analytes are measured from a blood sample, stool sample, urine sample, saliva or biopsy of an individual. In some embodiments, an individual's sample is extracted during fasting. A number of methods are known to extract samples from an individual and can be used within various embodiments of the invention. In several embodiments, analytes are extracted and measured at each time point, resulting in a dynamic analysis of the analytes.

In several embodiments, analytes are collected with periodicity across the timeline of pregnancy and postpartum. Accordingly, in some embodiments, analyte measurements are performed weekly, bi-weekly, monthly, per trimester, pre- and post-health event, after delivery, and any combination thereof. The precise extraction timeline will depend on the data to be collected and the model to be constructed.

A number of analytes can be used to determine gestational progress and/or gestational health, including (but not limited to) metabolites, protein constituents, genomic DNA, transcript expression, and lipids. In some embodiments, clinical data and/or personal data can be additionally used to determine gestational progress and/or gestational health. Analytes can be detected and measured by a number of methods, including nucleic acid and protein sequencing, mass spectrometry, colorimetric analysis, immunodetection, and the like. It should be noted that static, median, average, and/or dynamic analyte measurements can be used in accordance with various embodiments of the invention.

In numerous embodiments, an individual for use to derive data has been diagnosed as being pregnant, as determined by any appropriate method (e.g., ultrasonography). Embodiments are also directed to an individual being one that has not been diagnosed as pregnant.

A collection of individuals, in accordance with many embodiments, is a group of pregnant individuals to be measured so that their data can be used to construct and train a computational model. A collection will typically include individuals that are diagnosed as pregnant such that their analytes can be extracted along the pregnancy timeline. The number of individuals in a collection can vary, and in some embodiments, having a greater number of individuals will increase the prediction power of a trained computer model. The precise number and composition of individuals will vary, depending on the model to be constructed and trained.

Using the analyte measurements and gestational progress and/or gestational health, process 200 generates (203) training labels that provide a correspondence between analyte measurement features and gestational progress and/or gestational health. In several embodiments, analyte measurements used to generate training labels are determinative of gestational progress and/or gestational health. In some embodiments, analyte measurements are standardized.

Based on studies performed, it has been found that several analyte measurements provide robust predictive ability, including (but not limited to) metabolites, protein constituents, genomic DNA, transcript expression, and lipids. A number of methods can be used to select analyte measurements to be used as features in the training model. In some embodiments, correlation measurements between analyte measurements and gestational progress and/or gestational health are used to select features. In various embodiments, a computational model is used to determine which analyte measurements are best predictors. For example, a linear regression model (e.g., LASSO) or elastic net model can be used to determine which analyte measurement features provide the best predictive power as determined by their contribution.

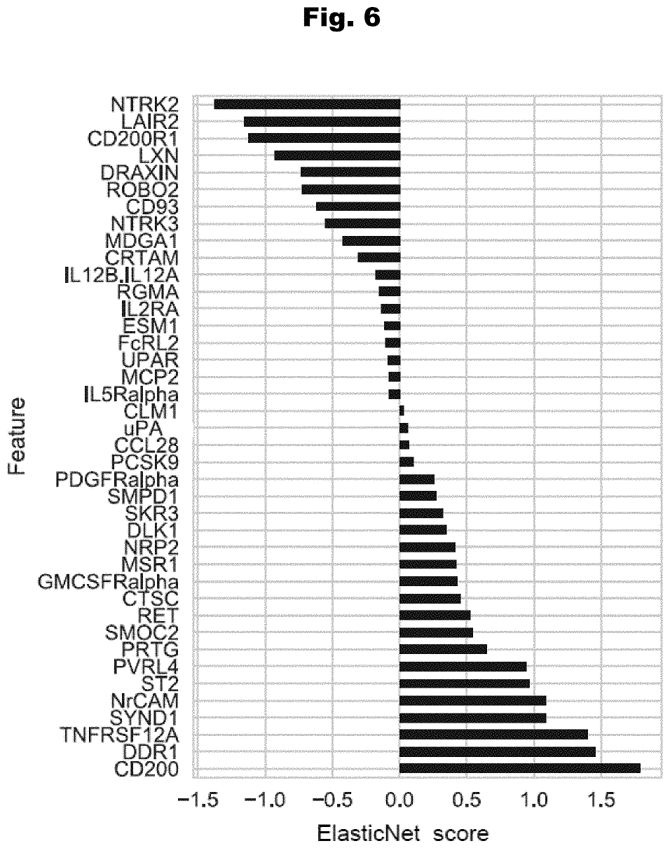

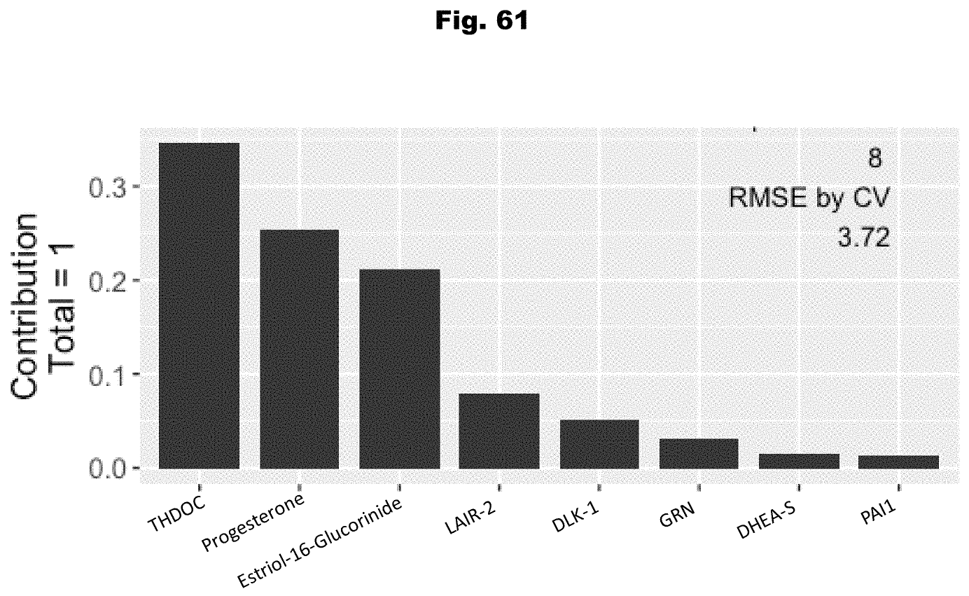

A selection of predictive analyte measurement features are described in the Exemplary Embodiments section (see Table 3 and FIG. 6). For instance, it has been found that the following 30 metabolites provide predictive power and can be utilized within a predictive model: N,N′-Dicarbobenzyloxy-L-ornithine, 1-(1Z-Hexadecenyl)-sn-glycero-3-phosphoethanolamine (PE(P-16:0e/0:0)), delta4-Dafachronic acid, C29H3609, 7alpha,24-Dihydroxy-4-cholesten-3-one, C22H43012P, C27H4409, C19H2807S, Androstane-3,17-diol, 21-Hydroxypregnenolone, Estriol-16-Glucuronide, C25H40O9, C27H44O4, C27H42O3, bilobol, [1-(3,5-dihydroxyphenyl)-12-hydroxytridecan-2-yl]acetate, C26H52NO8P, C27H4208, Prolylphenylalanine, N,N,Diacetyl-Lys-DAla-DAla, C23H49N2O5P, C21H29O, C33H53O9, C22H35O3, C30H44NO3S, 1,1′-(1,8-dioxo-1,8-octanediyl)bis[glycyl-glycine], C27H42010, 6-ketoestriol sulfate, DAH-3-Keto-4-en, and Progesterone. It is noted that two variations of progesterone, as detected mass spectrometry, were found to be predictive: progesterone (m/z: 315, RT/min: 9.3) and progesterone (m/z 337, RT/min 9.3) (see Table 3). In addition, 11 more metabolites unable to labeled by detectable by mass spectrometry were found to be predictive: (m/z: 511, RT/min: 5.4), (m/z: 519, RT/min: 8.6), (m/z: 563, RT/min: 6.6), (m/z: 353, RT/min: 7.9), (m/z: 487, RT/min: 6.6), (m/z: 319, RT/min: 2.6), (m/z: 821, RT/min: 9.1), (m/z: 653, RT/min: 9.3), (m/z: 798, RT/min: 8.5), (m/z: 260, RT/min: 9.8), and (m/z: 823, RT/min: 9.3). Likewise, it has been found that the following 42 protein constituents provide predictive power and can used in a predictive model: NTRK2, LAIR2, CD200R1, LXN, DRAXIN, ROBO2, CD93, NTRK3, MDGA1, CRTAM, IL12B/IL12A, RGMA, IL2RA, ESM1, FcRL2, UPAR, MCP2, IL5Ralpha, CLM1, uPA, CCL28, PCSK9, PDGFRalpha, SMPD1, SKR3, DLK1, NRP2, MSR1, GMCSFRalpha, CTSC, RET, SMOC2, PRTG, PVRL4, ST2, NrCAM, SYND1, TNFRSF12A, DDR1, CD200, GRN, and PAI1 (see FIGS. 6 and 61). Based on the foregoing, it should be understood that a number of combinations of analyte features can be used solitarily or combined in any fashion to be used to train a predictive computational model.

Training labels associating analyte measurement features and gestational progress and/or gestational health are used to construct and train (205) a computational model to determine an individual's gestational progress and/or gestational health. Various embodiments construct and train a model to determine the individual's pregnancy progression, time to delivery, and/or experiencing spontaneous abortion. A number of models can be used in accordance with various embodiments, including (but not limited to) ridge regression, K-nearest neighbors, LASSO regression, elastic net, least angle regression (LAR), random forest, and principal components analysis.

In several embodiments, computational models are built for dynamic observation. Accordingly, some embodiments of models incorporate analyte data of individuals at multiple time points across a pregnancy timeline such that the model can determine gestational progress across a pregnancy timeline selected. In some embodiments of models, a timeline is a full gestational timeline (i.e., from first missed menstruation or fertilization to birth) or a partial gestational timeline (e.g., first trimester, second trimester, third trimester). Various embodiments include postpartum analyte data and thus a timeline would include postpartum periods as well. It should be understood that any appropriate time period can be utilized in accordance with various embodiments of the invention.

In several embodiments, computational models can be built for static observation. Accordingly, some embodiments of models incorporate analyte data of individuals at a particular time point (or particular time points) of a pregnancy timeline (e.g., 4 weeks, 6 weeks, 8 weeks, 10 weeks, 12 weeks 16 weeks, 24 weeks, 28 weeks, 32 weeks, 36 weeks or 40 weeks). In some embodiments of models, a time point to be analyzed is related to time to birth (e.g., 1 week, 2 weeks, 3 weeks, 4 weeks, 6 weeks, or 8 weeks to birth). In some embodiments, a model incorporates analyte data related to a gestational event, especially events related to gestational health. Gestational events that can be modeled include delivery, spontaneous abortion, postpartum depression, gestational diabetes, gestational hypertension, gestational trophoblastic disease, preeclampsia, hyperemesis gravidarum (i.e., morning sickness), preterm labor or any other event that is related to gestation.

Models and sets of training labels used to train a model can be evaluated for their ability to accurately determine gestational progress and/or gestational health. By evaluating models, predictive abilities of analyte measurements can be confirmed. In some embodiments, a portion of the cohort data is withheld to test the model to determine its efficiency and accuracy. A number of accuracy evaluations can be performed, including (but not limited to) area under the receiver operating characteristics (AUROC), R-square error analysis, and mean square error analysis. In some embodiments, the contribution of each feature to the ability to predict outcome is determined. In some embodiments, top contributing features are utilized to construct the model. Accordingly, an optimized model can be identified.

Process 200 also outputs (207) the parameters of a computational model indicative of an individual's gestational age and/or gestational health from a panel of analyte measurements. Computational models can be used to determine an individual's gestational progress and/or gestational health, provide diagnoses, and treat an individual accordingly, as will be described in detail below.

While specific examples of processes for constructing and training a computational model to determine an individual's gestational progress and/or gestational health are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for constructing and training a computational model appropriate to the requirements of a given application can be utilized in accordance with various embodiments of the invention.

### Determination of an Individual's Pregnancy Progression and Potential Complications Using Analyte Measurements

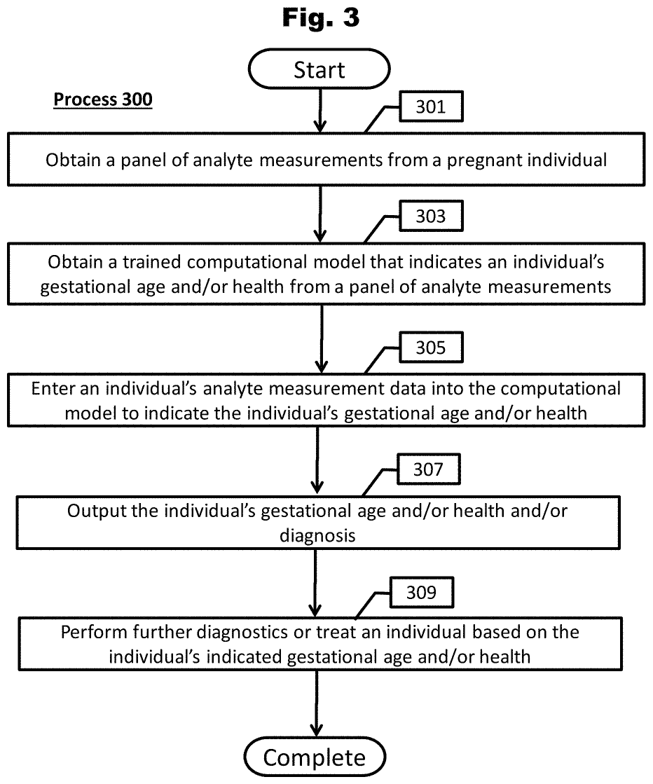

Once a computational model has been constructed and trained, it can be used to compute a determination of an individual's gestational progress and/or gestational health. As shown in FIG. 3, a method to determine an individual's gestational progress and/or gestational health using a trained computational model is provided in accordance with an embodiment of the invention. Process 300 obtains (301) a panel of analyte measurements from a pregnant individual.

In several embodiments, analytes are measured from a blood sample, stool sample, urine sample, saliva or biopsy of an individual. In some embodiments, an individual's sample is extracted during fasting. A number of methods are known to extract a sample from an individual and can be used within various embodiments of the invention. In several embodiments, analytes are extracted and measured at numerous time points, resulting in a dynamic analysis of the analytes. In some of these embodiments, analytes are measured with periodicity (e.g., weekly, monthly, trimester).

A number of analytes can be used to determine gestational progress and/or gestational health, including (but not limited to) metabolites, protein constituents, genomic DNA, transcript expression, and lipids. In some embodiments, clinical data and/or personal data can be additionally used to determine gestational progress and/or gestational health. Analytes can be detected and measured by a number of methods, including nucleic acid and protein sequencing, mass spectrometry, colorimetric analysis, immunodetection, and the like. It should be noted that static, median, average, and/or dynamic analyte measurements can be used in accordance with various embodiments of the invention. In many embodiments, the precise panel of analytes to be measured depends on the constructed and trained computational model to be used, as the input analyte measurement data that will be needed to at least partially overlap with the features used to train the model. That is, there should be enough overlap between the feature measurements used to train the model and the individual's analyte measurements obtained such that gestational progress and/or gestational health can be determined.

In numerous embodiments, an individual has been diagnosed as being pregnant, as determined by any appropriate method (e.g., ultrasonography or urine test). Embodiments are also directed to an individual being one that has not been diagnosed as pregnant, especially in situations in which the individual is unaware of her pregnancy.

Process 300 also obtains (303) a trained computational model that indicates an individual's gestational progress and/or gestational health from a panel of analyte measurements. Any computational model that can compute an indicator of an individual's gestational progress and/or gestational health from a panel of analyte measurements can be used. In some embodiments, the computational model is constructed and trained as described in FIG. 2. The computational model, in accordance with various embodiments, has been optimized to accurately and efficiently indicate gestational progress and/or gestational health.

A number of models can be used in accordance with various embodiments, including (but not limited to) ridge regression, K-nearest neighbors, LASSO regression, elastic net, least angle regression (LAR), random forest, and principal components analysis.

Process 300 also enters (305) an individual's analyte measurement data into a computational model to indicate the individual's gestational progress and/or gestational health. In some embodiments, the analyte measurement data is used to compute an individual's gestational progress and/or gestational health in lieu of performing a traditional gestational analysis (e.g., ultrasonography). Various embodiments utilize the analyte measurement data and computational model in combination with a clinical diagnostic methods.

Based on studies performed, it has been found that several analyte measurements provide robust predictive ability, including (but not limited to) particular metabolites, protein constituents, genomic DNA, transcript expression, and lipids. A number of methods can be used to select analyte measurements to be used as features in the training model. In some embodiments, correlation measurements between analyte measurements and gestational progress and/or gestational health are used to select features. In various embodiments, a computational model is used to determine which analyte measurements are best predictors. For example, a linear regression model (e.g., LASSO) or elastic net model can be used to determine which analyte measurement features provide the best predictive power as determined by their contribution.

A selection of predictive analyte measurement features are described in the Exemplary Embodiments section. For instance, it has been found that the following 30 metabolites provide predictive power and can be utilized within a predictive model: N,N′-Dicarbobenzyloxy-L-ornithine, 1-(1Z-Hexadecenyl)-sn-glycero-3-phosphoethanolamine (PE(P-16:0e/0:0)), delta4-Dafachronic acid, C29H36O9, 7alpha,24-Dihydroxy-4-cholesten-3-one, C22H43O12P, C27H44O9, C19H28O7S, Androstane-3,17-diol, 21-Hydroxypregnenolone, Estriol-16-Glucuronide, C25H40O9, C27H44O4, C27H42O3, bilobol, [1-(3,5-dihydroxyphenyl)-12-hydroxytridecan-2-yl] acetate, C26H52NO8P, C27H42O8, Prolylphenylalanine, N,N,Diacetyl-Lys-DAla-DAla, C23H49N2O5P, C21H29O, C33H5309, C22H3503, C30H44NO3S, 1,1′-(1,8-dioxo-1,8-octanediyl)bis[glycyl-glycine], C27H42010, 6-ketoestriol sulfate, DAH-3-Keto-4-en, and progesterone. It is noted that two variations of progesterone, as detected mass spectrometry, were found to be predictive: progesterone (m/z: 315, RT/min: 9.3) and progesterone (m/z 337, RT/min 9.3) (see Table 3). In addition, 11 more metabolites unable to labeled by detectable by mass spectrometry were found to be predictive: (m/z: 511, RT/min: 5.4), (m/z: 519, RT/min: 8.6), (m/z: 563, RT/min: 6.6), (m/z: 353, RT/min: 7.9), (m/z: 487, RT/min: 6.6), (m/z: 319, RT/min: 2.6), (m/z: 821, RT/min: 9.1), (m/z: 653, RT/min: 9.3), (m/z: 798, RT/min: 8.5), (m/z: 260, RT/min: 9.8), and (m/z: 823, RT/min: 9.3). In some embodiments, a gestation age prediction model includes measurements of at least one of the listed metabolites. In some embodiments, a gestation age prediction model includes measurements of at least two of the listed metabolites. In some embodiments, a gestation age prediction model includes measurements of at least three of the listed metabolites. In some embodiments, a gestation age prediction model includes measurements of at least four of the listed metabolites. In some embodiments, a gestation age prediction model includes measurements of at least five of the listed metabolites. In some embodiments, a gestation age prediction model includes measurements of at least six of the listed metabolites. In some embodiments, a gestation age prediction model includes at least measurements of seven of the listed metabolites. In some embodiments, a gestation age prediction model includes measurements of at least eight of the listed metabolites. In some embodiments, a gestation age prediction model includes measurements of at least nine of the listed metabolites. In some embodiments, a gestation age prediction model includes measurements of at least 10 of the listed metabolites. In some embodiments, a gestation age prediction model includes measurements of at least 15 of the listed metabolites. In some embodiments, a gestation age prediction model includes measurements of at least 20 of the listed metabolites. In some embodiments, a gestation age prediction model includes measurements of at least 25 of the listed metabolites. In some embodiments, a gestation age prediction model includes measurements of at least 30 of the listed metabolites. In some embodiments, a gestation age prediction model includes measurements of at least 35 of the listed metabolites. In some embodiments, a gestation age prediction model includes measurements of at least 40 of the listed metabolites. In some embodiments, a gestation age prediction model includes measurements of at least 42 of the listed metabolites.

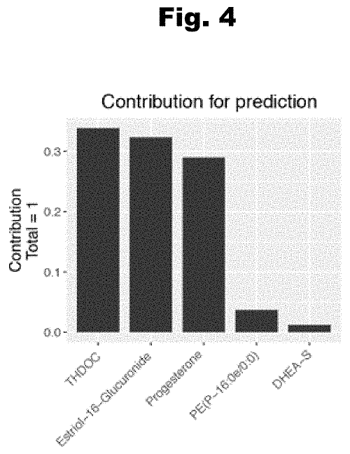

In one study, it was determined that tetrahydrodeoxycorticosterone (THDOC), estriol-16-glucoronide, progesterone, PE(P-16:0e/0:0), and dehydroepiandrosterone sulfate (DHEA-S) are high contributors for determining gestational age (FIG. 4; see Exemplary Embodiments). Accordingly, various embodiments are directed to models to predict gestational age (between 5 and 42 weeks) that utilize measurements one or more of the following analytes: THDOC, estriol-16-glucoronide, progesterone, PE(P-16:0e/0:0), DHEA-S, or any combination thereof.

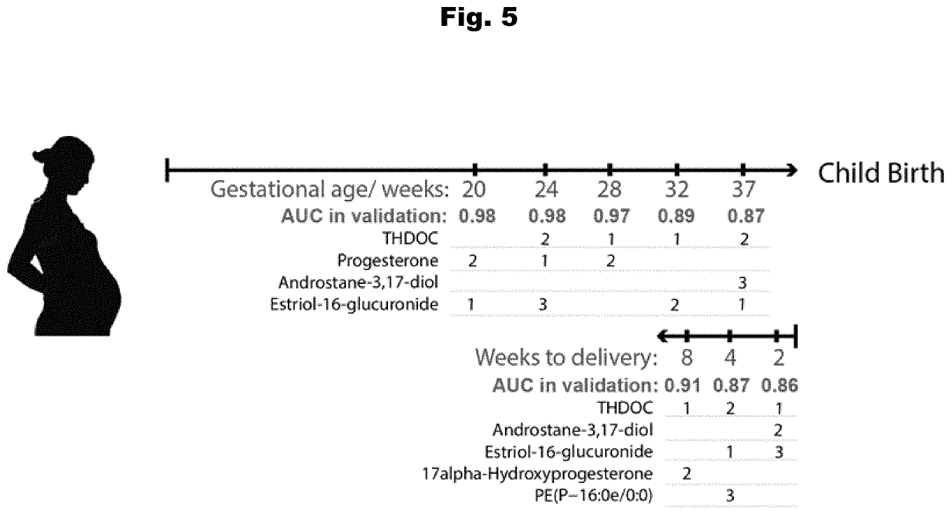

A number of analytes have been found to be predictive of particular gestational age time points (FIG. 5; see Exemplary Embodiments). Accordingly, various embodiments are directed to models to predict gestational age of 20 weeks that utilize measurements of one or more of the following analytes: estriol-16-glucoronide, progesterone, or any combination thereof. Various embodiments are directed to models to predict gestational age of 24 weeks that utilize measurements of one or more of the following analytes: THDOC, estriol-16-glucoronide, progesterone, or any combination thereof. Various embodiments are directed to models to predict gestational age of 28 weeks that utilize measurements of one or more of the following analytes: THDOC, progesterone, or any combination thereof. Various embodiments are directed to models to predict gestational age of 32 weeks that utilize measurements of one or more of the following analytes: THDOC, estriol-16-glucoronide or any combination thereof. Various embodiments are directed to models to predict gestational age of 37 weeks that utilize measurements of one or more of the following analytes: THDOC, estriol-16-glucoronide, androstane-3,17-diol, or any combination thereof. Various embodiments are directed to models to predict 8 weeks to delivery that utilize measurements of one or more of the following analytes: THDOC, alpha-hydroxyprogesterone, or any combination thereof. Various embodiments are directed to models to predict 4 weeks to delivery that utilize measurements of one or more of the following analytes: THDOC, estriol-16-glucoronide, PE(P-16:0e/0:0), or any combination thereof. Various embodiments are directed to models to predict 2 weeks to delivery that utilize measurements of one or more of the following analytes: THDOC, estriol-16-glucoronide, androstane-3,17-diol, or any combination thereof.

Likewise, a number of protein constituents have been found to be predictive of gestational (FIG. 6). Accordingly, various embodiments are directed to models to predict gestational age (between 5 and 42 weeks) that utilize measurements one or more of the following protein constituents: NTRK2, LAIR2, CD200R1, LXN, DRAXIN, ROBO2, CD93, NTRK3, MDGA1, CRTAM, IL12B/IL12A, RGMA, IL2RA, ESM1, FcRL2, UPAR, MCP2, IL5Ralpha, CLM1, uPA, CCL28, PCSK9, PDGFRalpha, SMPD1, SKR3, DLK1, NRP2, MSR1, GMCSFRalpha, CTSC, RET, SMOC2, PRTG, PVRL4, ST2, NrCAM, SYND1, TNFRSF12A, DDR1, CD200, GRN, PAI1 or any combination thereof. In some embodiments, a gestation age prediction model includes measurements of at least two of the listed protein constituents. In some embodiments, a gestation age prediction model includes measurements of at least three of the listed protein constituents. In some embodiments, a gestation age prediction model includes measurements of at least four of the listed protein constituents. In some embodiments, a gestation age prediction model includes measurements of at least five of the listed protein constituents. In some embodiments, a gestation age prediction model includes measurements of at least six of the listed protein constituents. In some embodiments, a gestation age prediction model includes measurements of at least seven of the listed protein constituents. In some embodiments, a gestation age prediction model includes measurements of at least eight of the listed protein constituents. In some embodiments, a gestation age prediction model includes measurements of at least nine of the listed protein constituents. In some embodiments, a gestation age prediction model includes measurements of at least 10 of the listed protein constituents. In some embodiments, a gestation age prediction model includes measurements of at least 15 of the listed protein constituents. In some embodiments, a gestation age prediction model includes measurements of at least 20 of the listed protein constituents. In some embodiments, a gestation age prediction model includes measurements of at least 25 of the listed protein constituents. In some embodiments, a gestation age prediction model includes measurements of at least 30 of the listed protein constituents. In some embodiments, a gestation age prediction model includes measurements of at least 35 of the listed protein constituents. In some embodiments, a gestation age prediction model includes measurements of at least 40 of the listed protein constituents. In some embodiments, a gestation age prediction model includes measurements of at least 42 of the listed protein constituents.

In addition, combining metabolite and protein constituent features have been found to be predictive of gestational (FIG. 61). Accordingly, various embodiments are directed to models to predict gestational age (between 5 and 42 weeks) that utilize measurements one or more of the metabolite and protein constituent analytes described above. Various embodiments are directed to models to predict gestational age (between 5 and 42 weeks) that utilize measurements one or more of the following analytes: THDOC, progesterone, estriol-16-glucoronide, LAIR2, DLK-1, GRN, DHEA-S, PAI1 or any combination thereof. In some embodiments, a gestation age prediction model includes measurements of at least two of the listed analytes. In some embodiments, a gestation age prediction model includes measurements of at least three of the listed analytes. In some embodiments, a gestation age prediction model includes measurements of at least four of the listed analytes. In some embodiments, a gestation age prediction model includes measurements of at least five of the listed analytes. In some embodiments, a gestation age prediction model includes measurements of at least six of the listed analytes. In some embodiments, a gestation age prediction model includes measurements of at least seven of the listed analytes. In some embodiments, a gestation age prediction model includes measurements of at least all eight of the listed analytes.

Process 300 also outputs (307) a report containing an individual's gestational age, weeks to delivery, and/or gestational health result and/or diagnosis. Furthermore, based on an individual's indicated gestational progress and/or gestational health, the individual is further examined and/or treated (309) to ameliorate a symptom related to the result and/or diagnosis. In several embodiments, an individual is provided with a personalized treatment plan. Further discussion of treatments that can be utilized in accordance with this embodiment are described in detail below, which may include various medications, dietary supplements, and surgical procedures.

While specific examples of processes for determining an individual's gestational progress and/or gestational health are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for computing an individual's gestational progress and/or gestational health appropriate to the requirements of a given application can be utilized in accordance with various embodiments of the invention.

### Feature Selection

As explained in the previous sections, analyte measurements are used as features to construct a computational model that is then used to indicate an individual's gestational progress and/or gestational health. Analyte measurement features used to train the model can be selected by a number of ways. In some embodiments, analyte measurement features are determined by which measurements provide strong correlation with gestational progress and/or gestational health. In various embodiments, analyte measurement features are determined using a computational model, such as Bayesian network, which can determine which analyte measurements influence or are influenced by an individual's gestational progress and/or gestational health. Embodiments also consider practical factors, such as (for example) the ease and/or cost of obtaining the analyte measurement, patient comfort when obtaining the analyte measurement, and current clinical protocols are also considered when selecting features.

Correlation analysis utilizes statistical methods to determine the strength of relationships between two measurements. Accordingly, a strength of relationship between an analyte measurement and gestational progress and/or gestational health can be determined. Many statistical methods are known to determine correlation strength (e.g., correlation coefficient), including linear association (Pearson correlation coefficient), Kendall rank correlation coefficient, and Spearman rank correlation coefficient. Analyte measurements that correlate strongly with gestational progress and/or gestational health can then be used as features to construct a computational model to determine an individual's gestational progress and/or gestational health.

In a number of embodiments, analyte measurement features are identified by a computational model, including (but not limited to) a Bayesian network model, LASSO, and elastic net. In some embodiments, the contribution of a feature to the predictive ability of the model is determined and features are selected based on their contribution. In some embodiments, the top contributing features are utilized. In some embodiments, the features that contribute over a percentage are selected (e.g., each feature that contributes at least 1% or the combination of top features that provide 90% contribution). In various embodiments, features that contribute at least 0.1%, 0.5%, 1%, 2%, 3%, 4%, 5%, or 10% to outcome prediction are selected. In various embodiments, the top features that in combination provide at least 50%, 75%, 80%, 90%, 95%, 99%, 99.5%, or 99.9% to outcome prediction are selected. The precise number of contributing features will depend on the results of the model and each feature's contribution. Various embodiments utilize an appropriate computational model that results in a number of features that is manageable. For instance, constructing predictive models from hundreds to thousands of analyte measurement features may have overfitting issues. Likewise, too few features can result in less prediction power.

### Biomarkers as Indicators of Gestation Age and Health

In several embodiments, biomarkers are detected and measured, and based on the ability to be detected and/or level of the biomarker, gestational progress and/or gestational health can be determined directly or via a computational model. Biomarkers that can be used in the practice of the invention include (but are not limited to) metabolites, protein constituents, genomic DNA, transcript expression, and lipids. As discussed in the Exemplary embodiments, a number of biomarkers have been found to be useful to determine gestational progress and/or gestational health, including (but not limited to) N,N′-Dicarbobenzyloxy-L-omithine, 1-(1Z-Hexadecenyl)-sn-glycero-3-phosphoethanolamine (PE(P-16:0e/0:0)), delta4-Dafachronic acid, C29H3609, 7alpha,24-Dihydroxy-4-cholesten-3-one, C22H43012P, C27H4409, C19H2807S, Androstane-3,17-diol, 21-Hydroxypregnenolone, Estriol-16-Glucuronide, C25H4009, C27H4404, C27H4203, bilobol, [1-(3,5-dihydroxyphenyl)-12-hydroxytridecan-2-yl]acetate, C26H52NO8P, C27H4208, Prolylphenylalanine, N,N,Diacetyl-Lys-DAla-DAla, C23H49N205P, C21H290, C33H5309, C22H3503, C30H44NO3S, 1,1′-(1,8-dioxo-1,8-octanediyl)bis[glycyl-glycine], C27H42010, 6-ketoestriol sulfate, DAH-3-Keto-4-en, and Progesterone. It is noted that two variations of progesterone, as detected mass spectrometry, were found to be predictive: progesterone (m/z: 315, RT/min: 9.3) and progesterone (m/z 337, RT/min 9.3) (see Table 3). In addition, 11 more metabolites unable to labeled by detectable by mass spectrometry were found to be predictive: (m/z: 511, RT/min: 5.4), (m/z: 519, RT/min: 8.6), (m/z: 563, RT/min: 6.6), (m/z: 353, RT/min: 7.9), (m/z: 487, RT/min: 6.6), (m/z: 319, RT/min: 2.6), (m/z: 821, RT/min: 9.1), (m/z: 653, RT/min: 9.3), (m/z: 798, RT/min: 8.5), (m/z: 260, RT/min: 9.8), and (m/z: 823, RT/min: 9.3). In addition, a number of protein constituent biomarkers have been found to be useful to determine gestational progress and/or gestational health, including (but not limited to) NTRK2, LAIR2, CD200R1, LXN, DRAXIN, ROBO2, CD93, NTRK3, MDGA1, CRTAM, IL12B/IL12A, RGMA, IL2RA, ESM1, FcRL2, UPAR, MCP2, IL5Ralpha, CLM1, uPA, CCL28, PCSK9, PDGFRalpha, SMPD1, SKR3, DLK1, NRP2, MSR1, GMCSFRalpha, CTSC, RET, SMOC2, PRTG, PVRL4, ST2, NrCAM, SYND1, TNFRSF12A, DDR1, CD200, GRN and PAI1.

### Detecting and Measuring Levels of Biomarkers

Analyte biomarkers in a biological sample (e.g., blood extraction, stool sample, urine sample, saliva, or biopsy) can be determined by a number of suitable methods. Suitable methods include chromatography (e.g., high-performance liquid chromatography (HPLC), gas chromatography (GC), liquid chromatography (LC)), mass spectrometry (e.g., MS, MS-MS), NMR, enzymatic or biochemical reactions, immunoassay, and combinations thereof. For example, mass spectrometry can be combined with chromatographic methods, such as liquid chromatography (LC), gas chromatography (GC), or electrophoresis to separate the metabolite being measured from other components in the biological sample. See, e.g., Hyotylainen (2012) Expert Rev. Mol. Diagn. 12(5):527-538; Beckonert et al. (2007) Nat. Protoc. 2(11):2692-2703; O'Connell (2012) Bioanalysis 4(4):431-451; and Eckhart et al. (2012) Clin. Transl. Sci. 5(3):285-288; the disclosures of which are herein incorporated by reference. Alternatively, analytes can be measured with biochemical or enzymatic assays. For example, glucose can be measured with a hexokinase-glucose-6-phosphate dehydrogenase coupled enzyme assay. In another example, biomarkers can be separated by chromatography and relative levels of a biomarker can be determined from analysis of a chromatogram by integration of the peak area for the eluted biomarker.

Immunoassays based on the use of antibodies that specifically recognize a biomarker may be used for measurement of biomarker levels. Such assays include (but are not limited to) enzyme-linked immunosorbent assay (ELISA), radioimmunoassays (RIA), “sandwich” immunoassays, fluorescent immunoassays, enzyme multiplied immunoassay technique (EMIT), capillary electrophoresis immunoassays (CEIA), immunoprecipitation assays, western blotting, immunohistochemistry (IHC), flow cytometry, and cytometry by time of flight (CyTOF).

Antibodies that specifically bind to a biomarker can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). A biomarker antigen can be used to immunize a mammal, such as a mouse, rat, rabbit, guinea pig, monkey, or human, to produce polyclonal antibodies. If desired, a biomarker antigen can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface-active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to a biomarker antigen can be prepared using any technique which provides for the production of antibody molecules by continuous cell lines in culture. These techniques include, but are not limited to, the hybridoma technique, the human B cell hybridoma technique, and the EBV hybridoma technique (Kohler et al., Nature 256, 495-97, 1985; Kozbor et al., J. Immunol. Methods 81, 31 42, 1985; Cote et al., Proc. Natl. Acad. Sci. 80, 2026-30, 1983; Cole et al., Mol. Cell Biol. 62, 109-20, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81, 6851-55, 1984; Neuberger et al., Nature 312, 604-08, 1984; Takeda et al., Nature 314, 452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grating of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332. Human monoclonal antibodies can be prepared in vitro as described in Simmons et al., PLoS Medicine 4(5), 928-36, 2007.

Alternatively, techniques described for the production of single chain antibodies can be adapted using methods known in the art to produce single chain antibodies which specifically bind to a particular antigen. Antibodies with related specificity, but of distinct idiotypic composition, can be generated by chain shuffling from random combinatorial immunoglobin libraries (Burton, Proc. Natl. Acad. Sci. 88,11120-23, 1991).

Single-chain antibodies also can be constructed using a DNA amplification method, such as PCR, using hybridoma cDNA as a template (Thirion et al., Eur. J. Cancer Prev. 5, 507-11, 1996). Single-chain antibodies can be mono- or bispecific, and can be bivalent or tetravalent. Construction of tetravalent, bispecific single-chain antibodies is taught, for example, in Coloma & Morrison, Nat. Biotechnol. 15, 159-63, 1997. Construction of bivalent, bispecific single-chain antibodies is taught in Mallender & Voss, J. Biol. Chem. 269,199-206,1994.

A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., Int. J Cancer 61, 497-501, 1995; Nicholls et al., J. Immunol. Meth. 165, 81-91, 1993).

Antibodies which specifically bind to a biomarker antigen also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared.

Antibodies can be purified by methods well known in the art. For example, antibodies can be affinity purified by passage over a column to which the relevant antigen is bound. The bound antibodies can then be eluted from the column using a buffer with a high salt concentration.

Antibodies may be used in diagnostic assays to detect the presence or for quantification of the biomarkers in a biological sample. Such a diagnostic assay may comprise at least two steps; (i) contacting a biological sample with the antibody, wherein the sample is blood or plasma, a microchip (e.g., See Kraly et al. (2009) Anal Chim Acta 653(1):23-35), or a chromatography column with bound biomarkers, etc.; and (ii) quantifying the antibody bound to the substrate. The method may additionally involve a preliminary step of attaching the antibody, either covalently, electrostatically, or reversibly, to a solid support, before subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The antibodies used in the diagnostic assays can be labeled with a detectable moiety. The detectable moiety should be capable of producing, either directly or indirectly, a detectable signal. For example, the detectable moiety may be a radioisotope, such as 2H, 14C, 32P, or 1251, a florescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating the antibody to the detectable moiety may be employed, including those methods described by Hunter et al., Nature, 144:945 (1962); David et al., Biochem. 13:1014 (1974); Pain et al., J. Immunol. Methods 40:219 (1981); and Nygren, J. Histochem. and Cytochem. 30:407 (1982).

Immunoassays can be used to determine the presence or absence of a biomarker in a sample as well as the quantity of a biomarker in a sample. First, a test amount of a biomarker in a sample can be detected using the immunoassay methods described above. If a biomarker is present in the sample, it will form an antibody-biomarker complex with an antibody that specifically binds the biomarker under suitable incubation conditions, as described above. The amount of an antibody-biomarker complex can be determined by comparing to a standard. A standard can be, e.g., a known compound or another protein known to be present in a sample. As noted above, the test amount of a biomarker need not be measured in absolute units, as long as the unit of measurement can be compared to a control.

In various embodiments, biomarkers in a sample can be separated by high-resolution electrophoresis, e.g., one or two-dimensional gel electrophoresis. A fraction containing a biomarker can be isolated and further analyzed by gas phase ion spectrometry. Preferably, two-dimensional gel electrophoresis is used to generate a two-dimensional array of spots for the biomarkers. See, e.g., Jungblut and Thiede, Mass Spectr. Rev. 16:145-162 (1997).

Two-dimensional gel electrophoresis can be performed using methods known in the art. See, e.g., Deutscher ed., Methods In Enzymology vol. 182. Typically, biomarkers in a sample are separated by, e.g., isoelectric focusing, during which biomarkers in a sample are separated in a pH gradient until they reach a spot where their net charge is zero (i.e., isoelectric point). This first separation step results in one-dimensional array of biomarkers. The biomarkers in the one-dimensional array are further separated using a technique generally distinct from that used in the first separation step. For example, in the second dimension, biomarkers separated by isoelectric focusing are further resolved using a polyacrylamide gel by electrophoresis in the presence of sodium dodecyl sulfate (SDS-PAGE). SDS-PAGE allows further separation based on molecular mass. Typically, two-dimensional gel electrophoresis can separate chemically different biomarkers with molecular masses in the range from 1000-200,000 Da, even within complex mixtures.

Biomarkers in the two-dimensional array can be detected using any suitable methods known in the art. For example, biomarkers in a gel can be labeled or stained (e.g., Coomassie Blue or silver staining). If gel electrophoresis generates spots that correspond to the molecular weight of one or more biomarkers of the invention, the spot can be further analyzed by densitometric analysis or gas phase ion spectrometry. For example, spots can be excised from the gel and analyzed by gas phase ion spectrometry. Alternatively, the gel containing biomarkers can be transferred to an inert membrane by applying an electric field. Then a spot on the membrane that approximately corresponds to the molecular weight of a biomarker can be analyzed by gas phase ion spectrometry. In gas phase ion spectrometry, the spots can be analyzed using any suitable techniques, such as MALDI or SELDI.

In a number of embodiments, high performance liquid chromatography (HPLC) can be used to separate a mixture of biomarkers in a sample based on their different physical properties, such as polarity, charge and size. HPLC instruments typically consist of a reservoir, the mobile phase, a pump, an injector, a separation column, and a detector. Biomarkers in a sample are separated by injecting an aliquot of the sample onto the column. Different biomarkers in the mixture pass through the column at different rates due to differences in their partitioning behavior between the mobile liquid phase and the stationary phase. A fraction that corresponds to the molecular weight and/or physical properties of one or more biomarkers can be collected. The fraction can then be analyzed by gas phase ion spectrometry to detect biomarkers.

After preparation, biomarkers in a sample are typically captured on a substrate for detection. Traditional substrates include antibody-coated 96-well plates or nitrocellulose membranes that are subsequently probed for the presence of biomarkers. Alternatively, metabolite-binding molecules attached to microspheres, microparticles, microbeads, beads, or other particles can be used for capture and detection of biomarkers. The metabolite-binding molecules may be antibodies, peptides, peptoids, aptamers, small molecule ligands or other metabolite-binding capture agents attached to the surface of particles. Each metabolite-binding molecule may comprise a “unique detectable label,” which is uniquely coded such that it may be distinguished from other detectable labels attached to other metabolite-binding molecules to allow detection of biomarkers in multiplex assays. Examples include, but are not limited to, color-coded microspheres with known fluorescent light intensities (see e.g., microspheres with xMAP technology produced by Luminex (Austin, Tex.); microspheres containing quantum dot nanocrystals, for example, having different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.); chemiluminescent dyes, combinations of dye compounds; and beads of detectably different sizes. See, e.g., U.S. Pat. Nos. 5,981,180, 7,445,844, 6,524,793, Rusling et al. (2010) Analyst 135(10): 2496-2511; Kingsmore (2006) Nat. Rev. Drug Discov. 5(4): 310-320, Proceedings Vol. 5705 Nanobiophotonics and Biomedical Applications II, Alexander N. Cartwright; Marek Osinski, Editors, pp. 114-122; Nanobiotechnology Protocols Methods in Molecular Biology, 2005, Volume 303; herein incorporated by reference in their entireties).

Mass spectrometry, and particularly SELDI mass spectrometry, is useful for detection of biomarkers. Laser desorption time-of-flight mass spectrometer can be used in embodiments of the invention. In laser desorption mass spectrometry, a substrate or a probe comprising biomarkers is introduced into an inlet system. The biomarkers are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of markers of specific mass to charge ratio.

Matrix-assisted laser desorption/ionization mass spectrometry (MALDI-MS) can also be used for detecting biomarkers. MALDI-MS is a method of mass spectrometry that involves the use of an energy absorbing molecule, frequently called a matrix, for desorbing proteins intact from a probe surface. MALDI is described, for example, in U.S. Pat. No. 5,118,937 (Hillenkamp et al.) and U.S. Pat. No. 5,045,694 (Beavis and Chait). In MALDI-MS, the sample is typically mixed with a matrix material and placed on the surface of an inert probe. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in this art. The matrix dries, forming crystals that encapsulate the analyte molecules. Then the analyte molecules are detected by laser desorption/ionization mass spectrometry.

Biomarkers on the substrate surface can be desorbed and ionized using gas phase ion spectrometry. Any suitable gas phase ion spectrometer can be used as long as it allows biomarkers on the substrate to be resolved. Preferably, gas phase ion spectrometers allow quantitation of biomarkers. In one embodiment, a gas phase ion spectrometer is a mass spectrometer. In a typical mass spectrometer, a substrate or a probe comprising biomarkers on its surface is introduced into an inlet system of the mass spectrometer. The biomarkers are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector. The detector then translates information of the detected ions into mass-to-charge ratios. Detection of the presence of biomarkers or other substances will typically involve detection of signal intensity. This, in turn, can reflect the quantity and character of biomarkers bound to the substrate. Any of the components of a mass spectrometer (e.g., a desorption source, a mass analyzer, a detector, etc.) can be combined with other suitable components described herein or others known in the art in embodiments of the invention.

The methods for detecting biomarkers in a sample have many applications. For example, the biomarkers are useful in monitoring women during pregnancy, for example to determine gestational age, predict time until delivery, or assess risk of spontaneous abortion.

### Kits

In several embodiments, kits are utilized for monitoring women during pregnancy, wherein the kits can be used to detect analyte biomarkers as described herein. For example, the kits can be used to detect any one or more of the analyte biomarkers described herein, which can be used to determine gestational age, predict time until delivery, and/or assess risk of spontaneous abortion. The kit may include one or more agents for detection of one or more metabolite biomarkers, a container for holding a biological sample (e.g., blood or plasma) obtained from a subject; and printed instructions for reacting agents with the biological sample to detect the presence or amount of one or more biomarkers in the sample. The agents may be packaged in separate containers. The kit may further comprise one or more control reference samples and reagents for performing a biochemical assay, enzymatic assay, immunoassay, or chromatography. In various embodiments, a kit may include an antibody that specifically binds to a biomarker. In some embodiments, a kit may contain reagents for performing liquid chromatography (e.g., resin, solvent, and/or column).

A kit can include one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of monitoring women during pregnancy, e.g., to determine gestational age, predict time until delivery, and/or predict imminent spontaneous abortion.

### Applications and Treatments Related to Gestational Progress and Health

Various embodiments are directed to performing further diagnostics and or treatments based on a determination of gestational progress and/or gestational health. As described herein, a pregnant individual's gestational progress and/or gestational health is determined by various methods (e.g., computational methods, biomarkers). Based on one's gestational progress and/or gestational health, an individual can be subjected to further diagnostic testing and/or treated with various medications, dietary supplements, and surgical procedures.

### Clinical Diagnostics, Medications and Supplements

Several embodiments are directed to the use of medications and/or dietary supplements to treat an individual based on their gestational progress and/or gestational health determination. In some embodiments, medications and/or dietary supplements are administered in a therapeutically effective amount as part of a course of treatment. As used in this context, to “treat” means to ameliorate at least one symptom of the disorder to be treated or to provide a beneficial physiological effect. For example, one such amelioration of a symptom could be improvement in gestational health. Assessment of gestational progress and/or gestational health can be performed in many ways, including (but not limited to) the use of analyte measurements and sonography.

A therapeutically effective amount can be an amount sufficient to prevent reduce, ameliorate or eliminate the symptoms of diseases or pathological conditions susceptible to such treatment, such as, for example, spontaneous abortion or other gestational disorders. In some embodiments, a therapeutically effective amount is an amount sufficient to improve gestational health or reduce the risk of spontaneous abortion.

Various embodiments are directed towards getting an indication of gestational progress and performing an intervention and/or treatment thereupon. In some embodiments, when a pregnant individual is experiencing various symptoms at various points of gestational age or timeline to pregnancy (as determined by methods described herein), an intervention and/or treatment is performed. In some embodiments, treatments are performed when an individual exhibits symptoms that occur early and/or late according a determined gestational age or timeline to delivery. For example, a pregnant individual experiencing regular contractions prior to 37 weeks is considered to be in premature (preterm) labor, and a number of interventions and/or treatments can be performed. Likewise, gestation periods of longer than 42 weeks is considered to be a postterm pregnancy, additional monitoring, induction of labor, and/or Caesarian delivery is performed to avoid complications.

In a number of embodiments, when a pregnant individual is experiencing regular contractions, a gestational age can be determined, which would indicate whether the individual is experiencing preterm labor. In some embodiments, a gestational age is determined prior to any experienced contractions (e.g., as determined during the course of pregnancy) and based on the determined gestational age, an indication of preterm labor is determined. In accordance with various embodiments, it may be desirable to confirm that an individual is in preterm labor, and thus confirmation of labor can be performed by a number of means, including (but not limited to) cervical exam, sonography, testing for amniotic fluid, testing for fetal fibronectin, or any combination thereof. Treatments for preterm labor include (but not limited to) intravenous fluids, antibiotics (to treat infection), tocolytic medications (to slow or stop contractions), antenatal corticosteroids (to help mature fetus), cervical cerclage (to close up cervix), delivery of the baby, or any appropriate combination thereof. Tocolytic medications include (but not limited to) indomethacin, magnesium sulfate, orciprenaline, ritodrine, terbutaline, salbutamol, nifedipine, fenoterol, nylidrin, isoxsuprine, hexoprenaline, and atosiban. Antenatal corticosteroids include (but not limited to) dexamethasone and betamethasone. For more on treatment and care of preterm labor, see J. N. Robinson and E. R. Norwitz. Ed.: V. A. Barss. UpToDate, retrieved September 2019 (https://www.uptodate.com/contents/preterm-birth-risk-factors-interventions-for-risk-reduction-and-maternal-prognosis); C. J. Lockwood. Ed.: V. A. Barss. UpToDate, retrieved September 2019 (https://www.uptodate.com/contents/preterm-labor-clinical-findings-diagnostic-evaluation-and-initial-treatment); and H. N. Simhan and S. Caritis. Ed.: V. A. Barss. UpToDate, retrieved September 2019 (https://www.uptodate.com/contents/inhibition-of-acute-preterm-labor): the disclosure of which are each incorporated herein by reference).

In several embodiments, a pregnancy may go beyond a gestational age of 42 weeks, as determined by various methods described herein. As gestational age exceeds 42 weeks, the placenta may age, begin deteriorating, or fail. Accordingly, a number of embodiments are directed towards determining a gestational age and determine whether the individual is in a postterm pregnancy. In some embodiments, when a postterm pregnancy is indicated, additional monitoring can be performed, including (but not limited to) fetal movement recording (to monitor regular movements of fetus), doppler fetal monitor (to measure fetal heart rate), nonstress test (to monitor fetal heartbeat) and Doppler flow study (to monitor blood flow in and out of placenta). In some embodiments, when a postterm pregnancy is indicated, labor is induced and/or Caesarian delivery is performed.

In many embodiments, the gestational age and time to delivery are determined and used concurrently to determine whether an individual will experience preterm labor or a postterm pregnancy. In some embodiments, a time to delivery equal to or less than a gestational age of 37 weeks is determined, indicating that preterm labor is likely and thus interventions and treatments for preterm labor are performed. Likewise, in some embodiments, a time to delivery equal to or more than a gestational age of 42 weeks is determined, indicating that a postterm pregnancy is likely and thus monitoring, induced labor, or Casesarian delivery are performed.

In a similar manner, interventions and/or treatments can be performed at various other time points, as would be understood in the art. Accordingly, various methods described herein can determine gestational progress and based on symptoms, can perform an interventions and/or treatments. Critical time points include gestational ages of 20 weeks for determination of successful pregnancy and mitigating miscarriage, 24 weeks for determination age of viability, 28 weeks for determination of extreme preterm labor, 32 weeks for very preterm labor, 37 weeks for preterm labor, and 42 weeks for postterm pregnancy. At each time point, various interventions include prenatal checkups and monitoring, including measuring blood pressure, checking for urinary tract infection, checking for signs of preeclampsia, checking for signs of gestational hypertension, checking for signs of gestational diabetes, checking for signs of preterm labor, checking for signs of preterm rupture of membranes, measure heartbeat of fetus, measure fundal height, look for swelling in hands or feet, sampling for chorionic villus, check for risk of genetic disorders (e.g., Down syndrome and spina bifida), perform amniocentesis test, sonography, determine baby gender, and performing blood tests (e.g., glucose screening, anemia, status of Rh-positive or -negative).

A number of medications are available to treat spontaneous abortion and include (but are not limited to) estrogens, and progestogens (e.g., progesterone, dydrogesterone), or a combination thereof.

Numerous dietary supplements may also help to treat risk of spontaneous abortion. Various dietary supplements, such as folic acid, iron, calcium, vitamin D, docosahexaenoic acid (DHA), and iodine have been shown to have beneficial effects on pregnancy and reducing gestational disorders including spontaneous abortion. Thus, embodiments are directed to the use of dietary supplements, included those listed herein, to be used to treat an individual based on one's gestational progress and/or gestational health result.

### Exemplary Embodiments

Bioinformatic and biological data support the methods and systems of assessing gestational progress and applications thereof. In the ensuing sections, exemplary methods and exemplary applications related to gestation that incorporate analyte panels, correlations, and computational models are provided.

### Example 1: Metabolomics and Human Pregnancy

Metabolomics, which profiles compounds constituting a biological system closest to a phenotype, is appreciated for its roles in making biomarker and mechanistic discoveries. For pregnancy-associated diseases, profiling of blood and urinary metabolites has uncovered novel biochemical molecules and pathways associated with preeclampsia, gestational diabetes and premature labor. However, to date, most profiling approaches have typically examined only small subsets of biomolecules at only one or a few time points during pregnancy. Within this example, untargeted metabolomics were used to systematically profile metabolites throughout pregnancy with an unprecedented weekly sampling of maternal blood. The total number of pregnancy-related metabolites and metabolic pathways identified offer a comprehensive view of the maternal-fetal metabolic adaptation. Panels including a small number of metabolic features from maternal blood that can predict the timing of pregnancy with high precision were identified.

### Research Design and Cohort

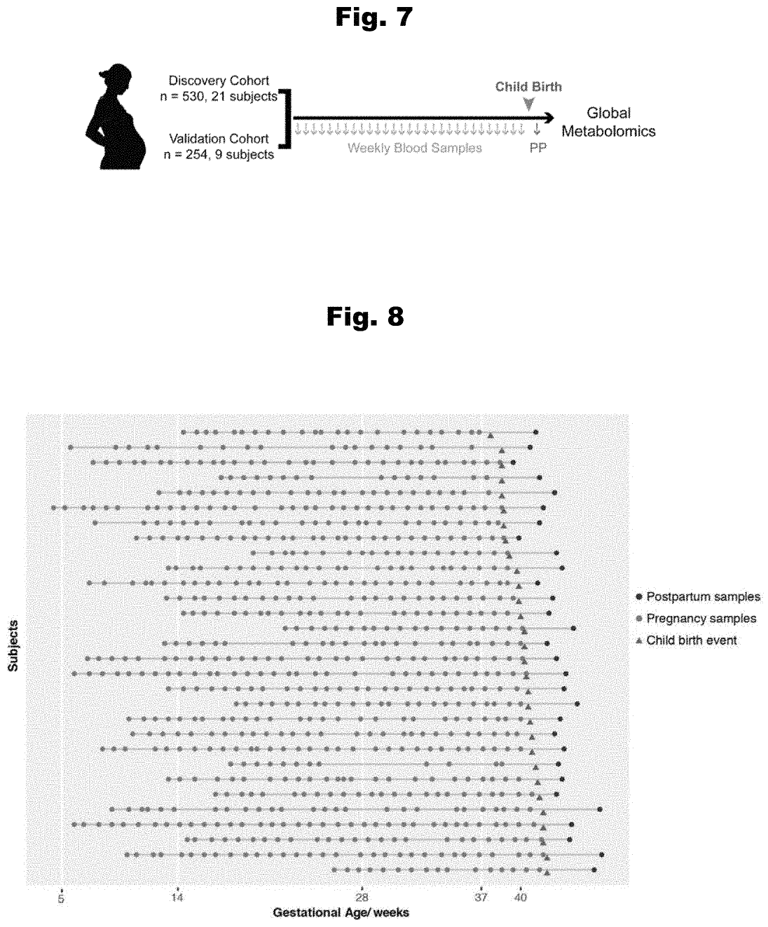

To capture the highly dynamic pregnancy process, a multi-year single-center Danish normal pregnancy cohort was established with a unique design of high-density blood sampling. Consented female participants submitted weekly blood draws beginning week 5 in pregnancy until postpartum. A total of 30 women with weekly blood sampling were assigned to a discovery (N=21) and a validation (N=9) cohort (Table 1, FIGS. 7 and 8), whose samples were analyzed in two separated years. In addition, another separate set of women (N=8) were included as the second validation cohort, in which samples were analyzed independently three years apart from the discovery cohort.

### Weekly Pregnancy Progression is Precisely Ordered by Metabolites

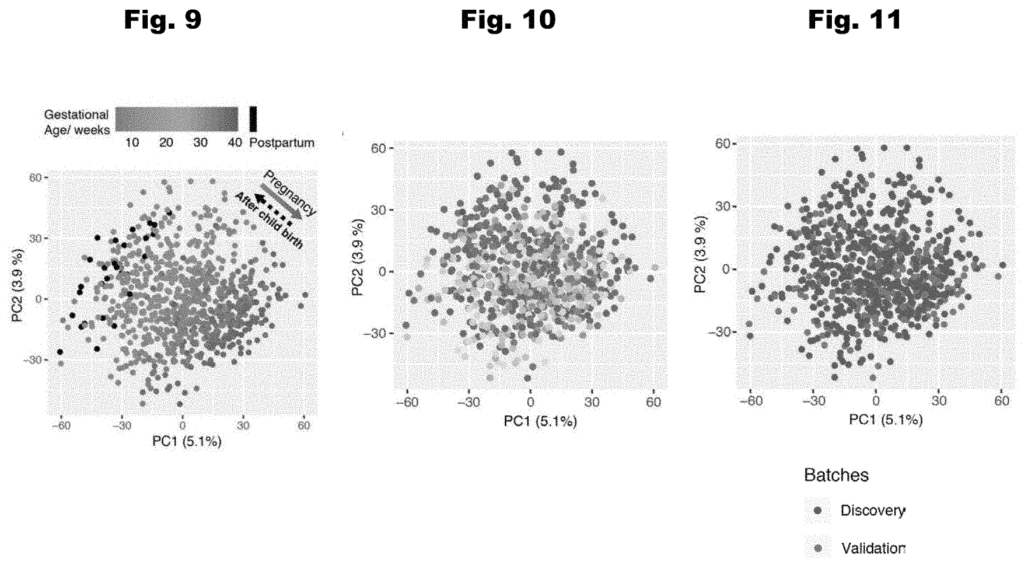

The 784 samples from 30 subjects were randomized within each cohort (discovery and validation), processed following a standard protocol, and analyzed by liquid chromatography-mass spectrometry (LC-MS) for untargeted metabolomics across two separate years (For protocol, see K. Contrepois, L. Jiang, and M. Snyder Mol. Cell Proteomics 14, 1684-1695 (2015), the disclosure of which is incorporated herein by refrence). After quality control, data filtering and normalization, 9,651 metabolic features were identified across the different samples, with 4,995 features (51.7%) altered during pregnancy and/or at postpartum (FDR<0.05). The data was globally examined with principal component analysis (PCA), in which the samples were distributed based on the first two principal components according to their gestational stages (FIG. 9), regardless of individual variation and batches (FIGS. 10 and 11). FIG. 9 provides PCA analysis of metabolite results according to gestational age (each data point represents a metabolite and colored by gestational age). FIG. 10 provides PCA metabolite results according to participant (each data point represents a metabolite and colored by individual). FIG. 11 provides PCA metabolite results according to batch testing (each data point represents a metabolite and colored by whether data was in discovery cohort or validation cohort).

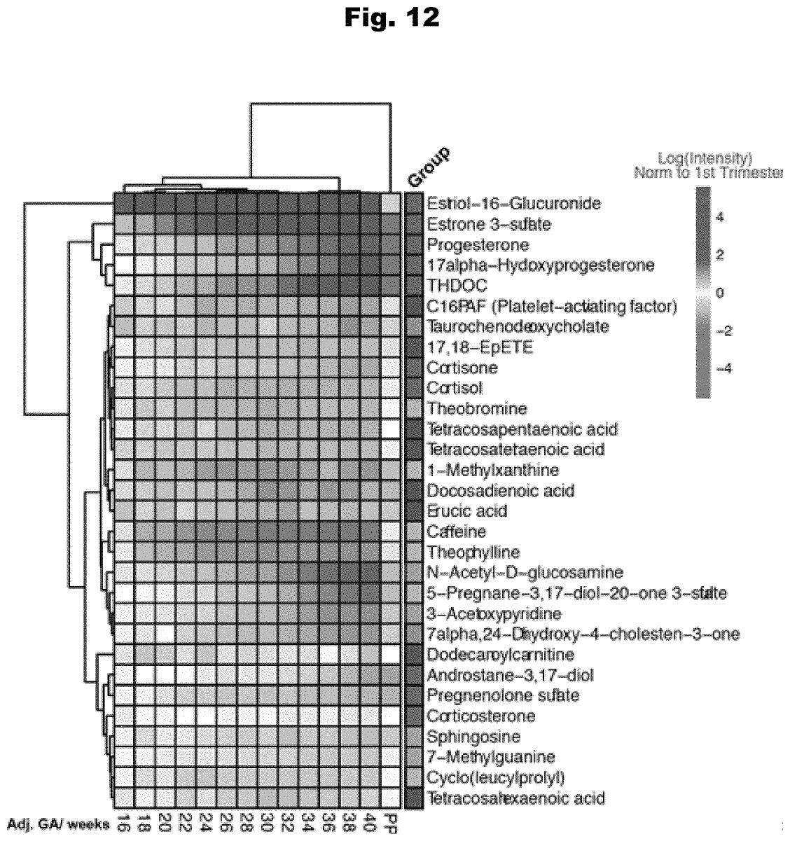

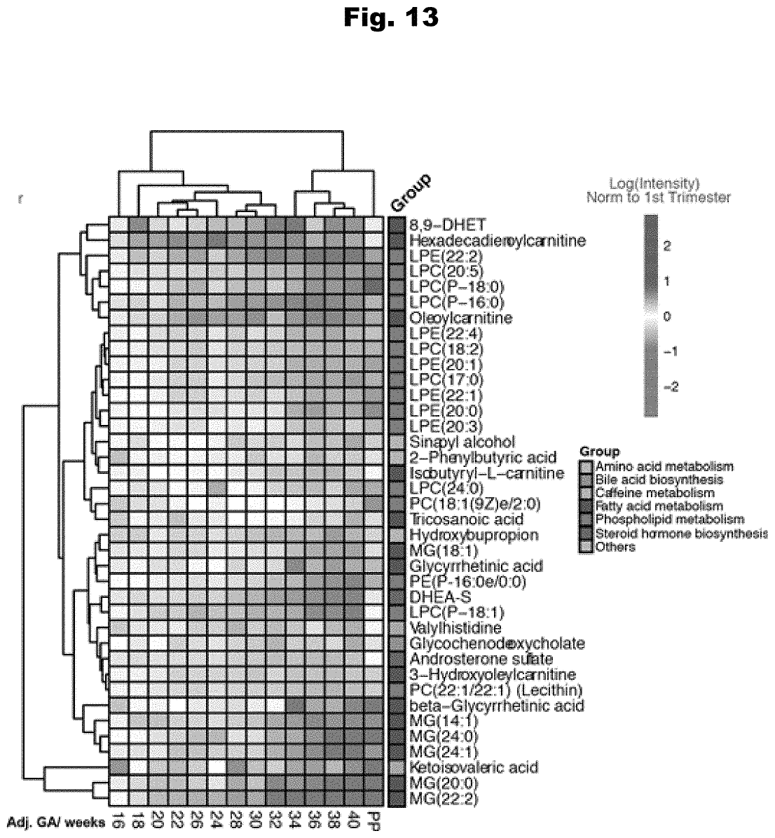

To understand the potential function of pregnancy-related metabolites, metabolic features were annotated using an in-house library and a combined public spectral databases. A total of 952 metabolic features were mapped to 687 compounds, which include plasma metabolites carrying out important functions in human. Among them, 460 compounds were significantly associated with pregnancy (70%, FDR<0.05, SAM). In addition, 264 compounds were identified with a MSI level 1 or 2, including 176 compounds (66.7%) that were significantly associated with pregnancy as determined by linear regression with gestational age, including well-known pregnancy-related metabolites such as progesterone and 17alpha-hydoxyprogesterone (FDR<0.05, SAM, FIGS. 12 and 13, Table 2). Hierarchical clustering of the weekly samples revealed a week order consistent with the actual gestational age progression (FIGS. 12 and 13). Together these results suggest a dramatic and programmed change of human blood metabolites at a system level during pregnancy.

### Metabolite Groups Altered During Pregnancy

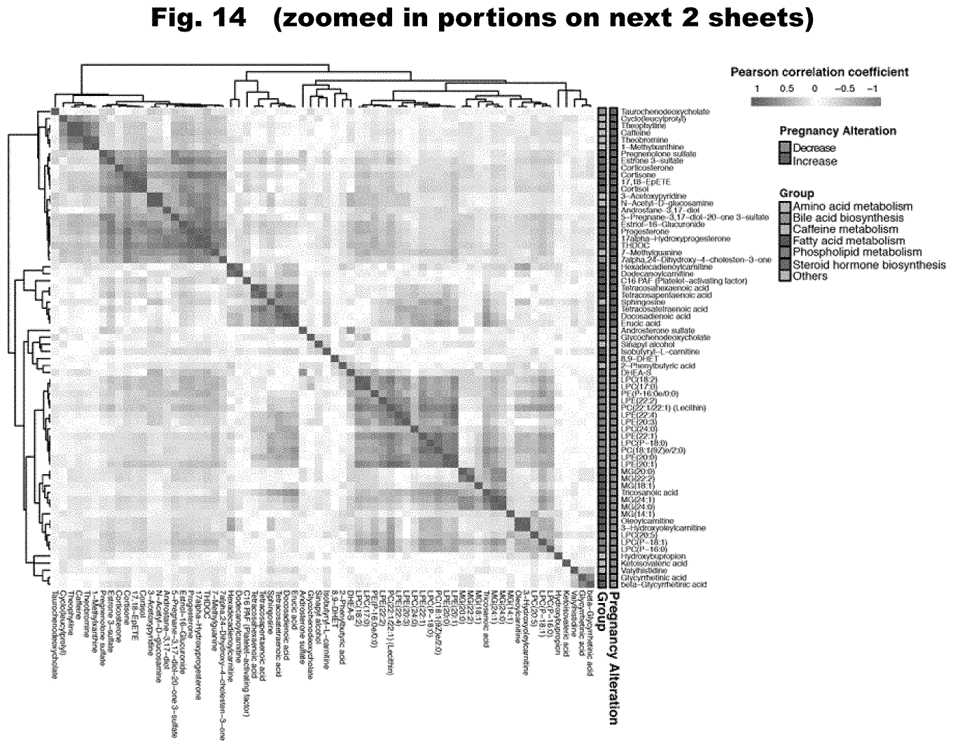

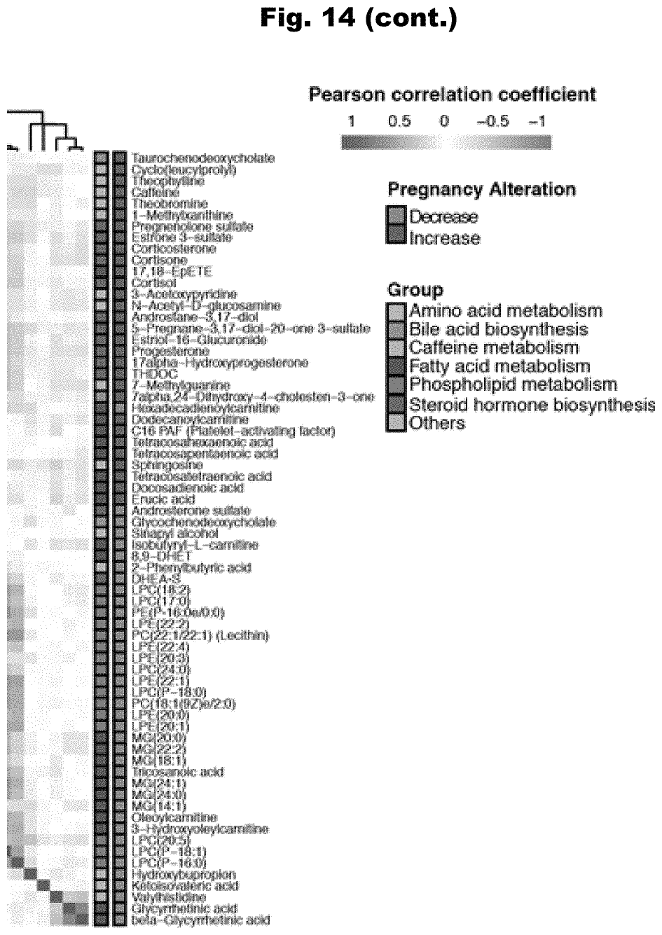

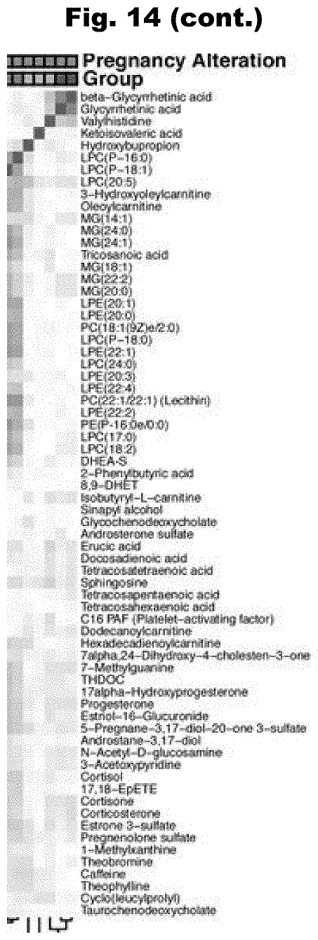

In order to detect the functional groups of metabolites altered during pregnancy, correlation analysis was performed on the intensities of the 68 top pregnancy-related compounds across all samples. In FIG. 14, metabolites that were significantly elevated (N=30) or decreased (n=38) tended to cluster together. Among them, known pregnancy-related steroid hormones were recognized, including progesterone, 17alpha-hydroxyprogesterone, and dehydroepiandrosterone sulfate (DHEA-S, FIG. 14).

Using existing structural and functional information, the pregnancy-related compounds were categorized into seven groups. These findings highlighted that even though the level of each compound is dynamically changing during pregnancy, a highly coordinated metabolite regulation existed underlying the pregnancy process.

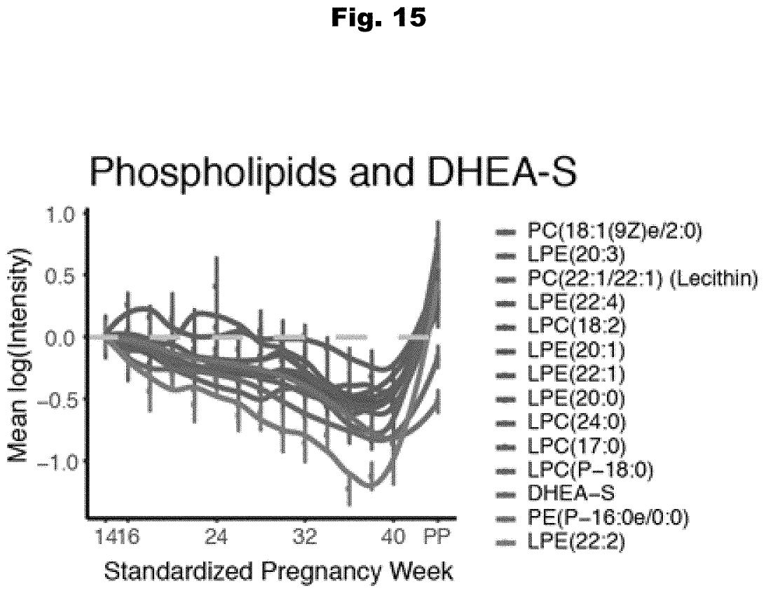

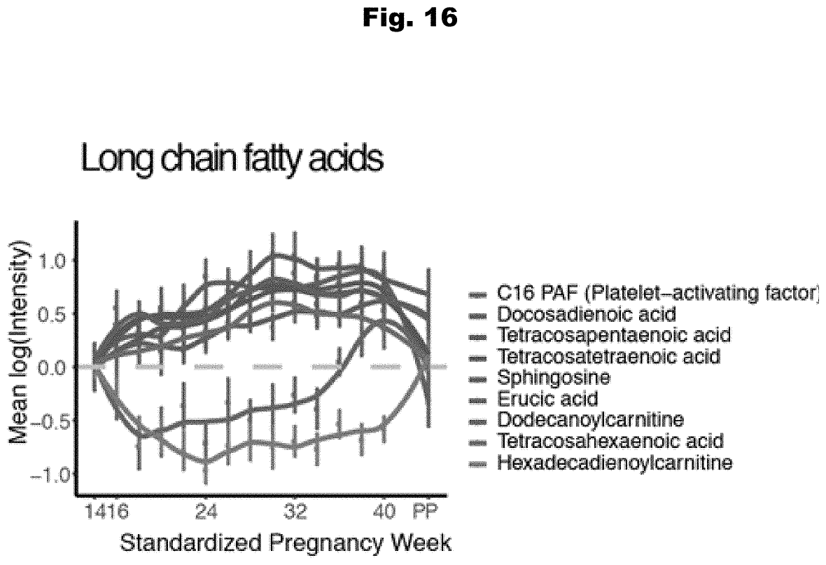

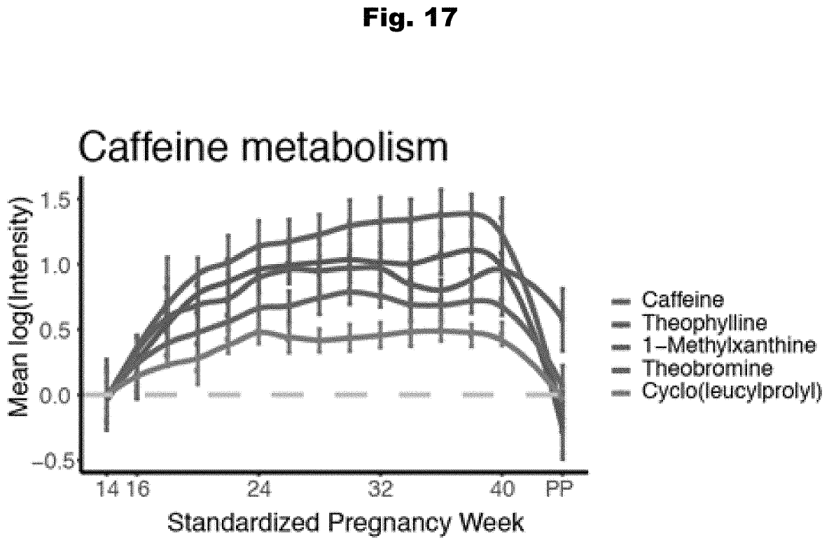

Within the lipid block, the intra-correlation was relatively high. The largest cluster was composed of lysophosphatidylcholines (LysoPCs), a subset of phospholipids, which gradually decreased during pregnancy and increased after childbirth (FIG. 15). LysoPCs are bioactive proinflammatory lipids that have been linked with organismal oxidative stress and inflammation. The second largest cluster of metabolites included a number of free fatty acids that were highly correlated (FIG. 16). Many long chain fatty acids showed dynamic changes in their level revealed by the dense sampling, with one wave of increase in the second and the third trimesters (FIG. 16). After childbirth, the levels of many long chain fatty acids decreased (FIG. 16). Within the non-lipid block, the intra-correlation was relatively weak. One cluster included five highly correlated metabolites belonging to the same caffeine metabolism pathway (FIG. 17). All five metabolites were consistently elevated during pregnancy, with caffeine reaching a level of concentration three times higher at the end than beginning of pregnancy (FIG. 17). Overall, among 89 pregnancy-related compounds identified, functional metabolite groups such as LysoPCs, fatty acids, and caffeine metabolism were altered in an orchestrated manner during pregnancy, with individual compounds within each group showing strong inter-correlation to each other.

### Orchestrated Metabolome Reconfigurations Span Multiple Pathways During Pregnancy

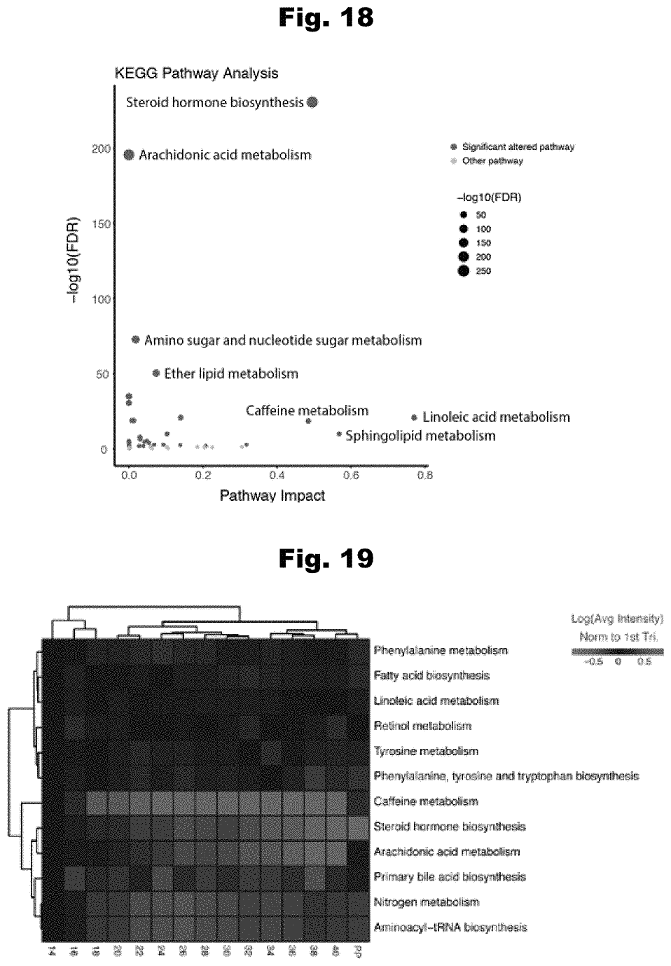

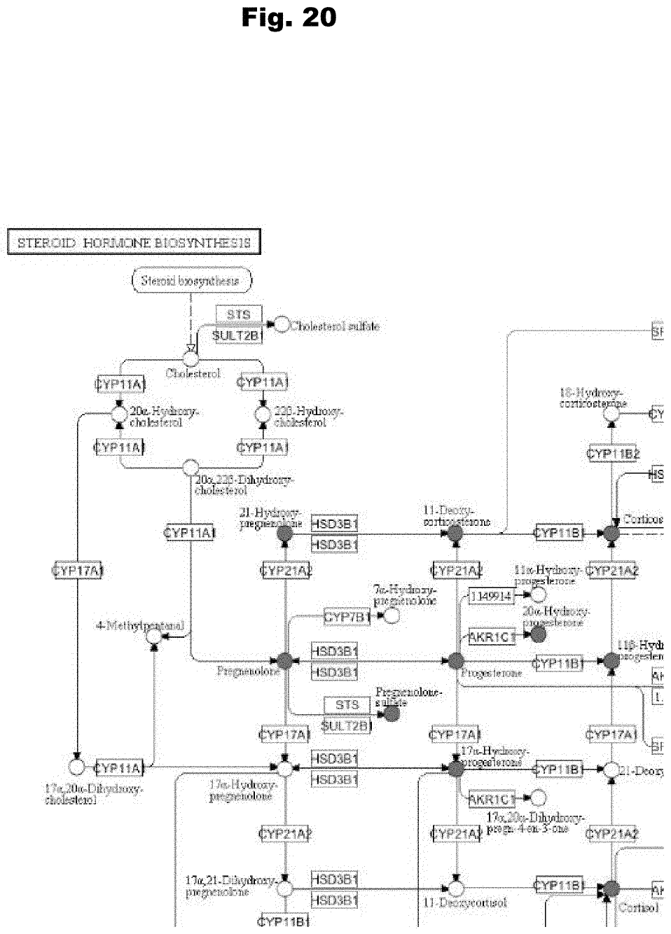

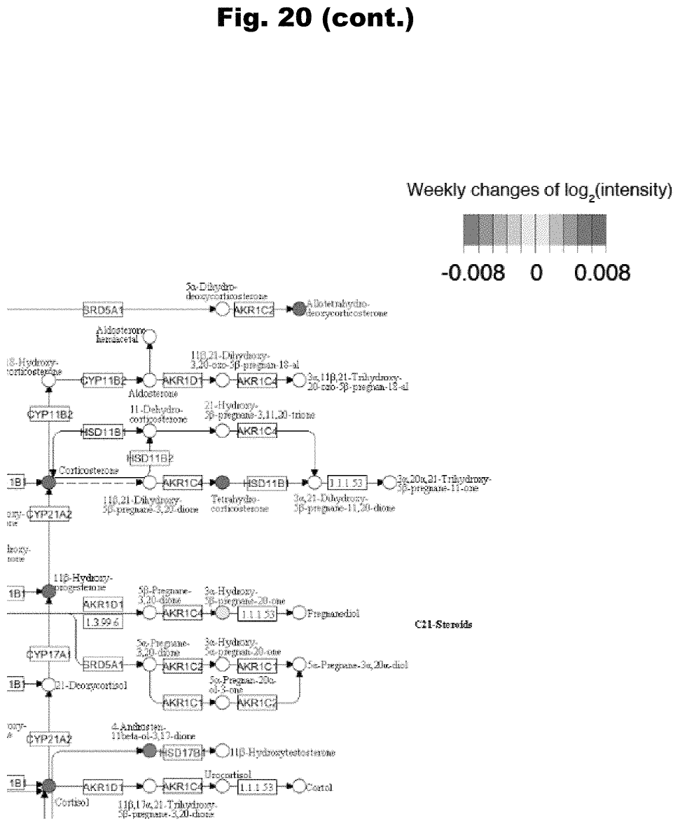

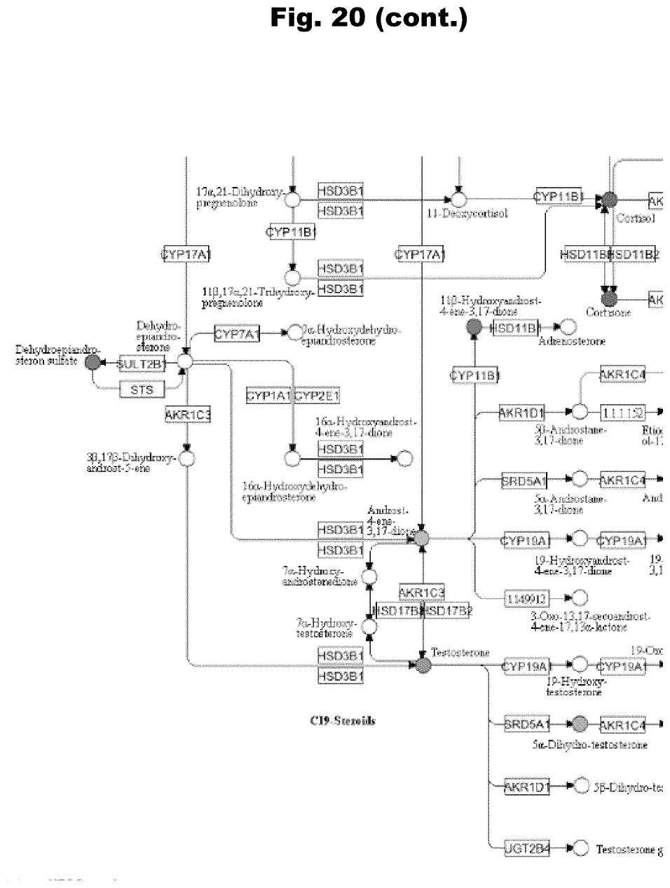

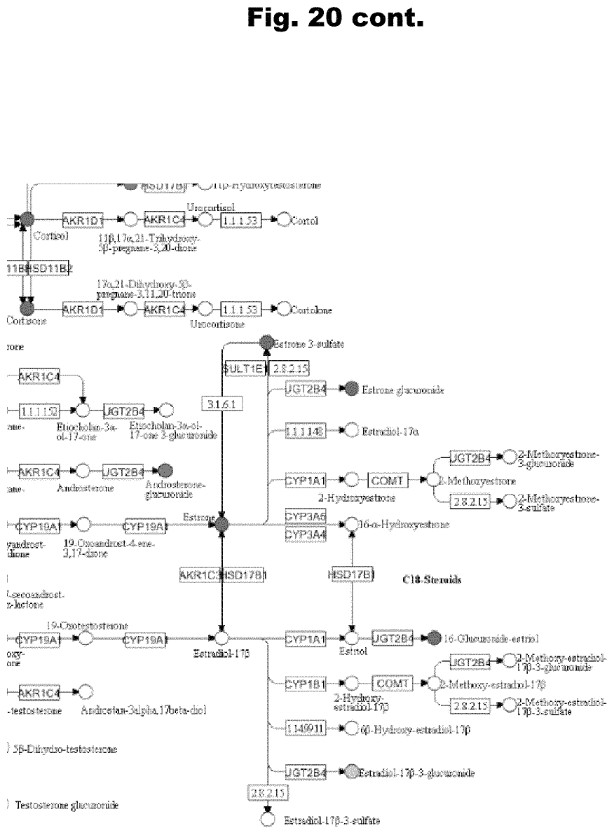

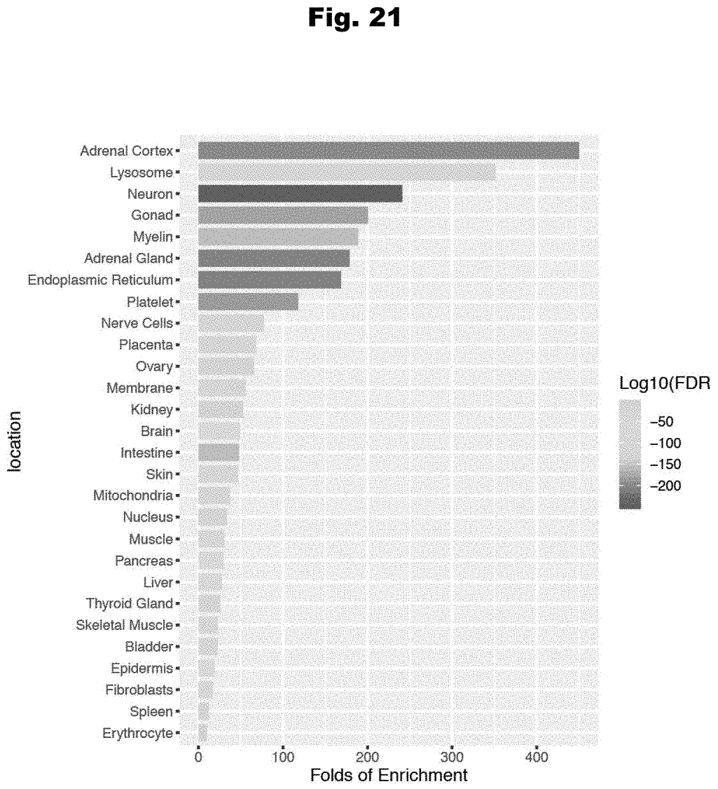

Next, the global pathway changes were examined during normal pregnancy. Among the 48 mapped KEGG pathways, 34 showed significant changes (70.8%, adjusted FDR<0.05, global test, FIG. 18), suggesting large scale pathway alterations of metabolism in pregnancy. To quantify the pathway activities through gestational age, the pathway-wise average intensity of metabolites was calculated. The analysis revealed the high-resolution dynamics of energy metabolism processes during pregnancy (FIG. 19). In addition, steroid hormone biosynthesis is the top altered pathways (FIG. 18). Along with the essential roles of steroid hormones in maintaining pregnancy and later inducing parturition, an orchestrated elevation of many components centered on progesterone in the pathway was observed (FIG. 20). Metabolite set enrichment analysis (MSEA) revealed that placenta and gonads were among the top origins of the pregnancy-related metabolites (FIG. 21). The ability to recognize numerous steroid hormones well-documented to change during pregnancy validates our approach.

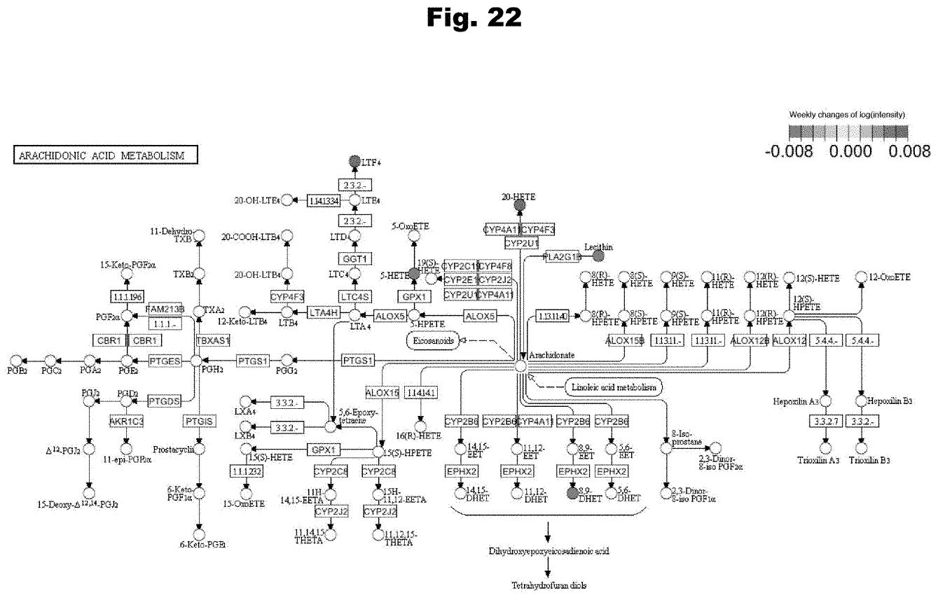

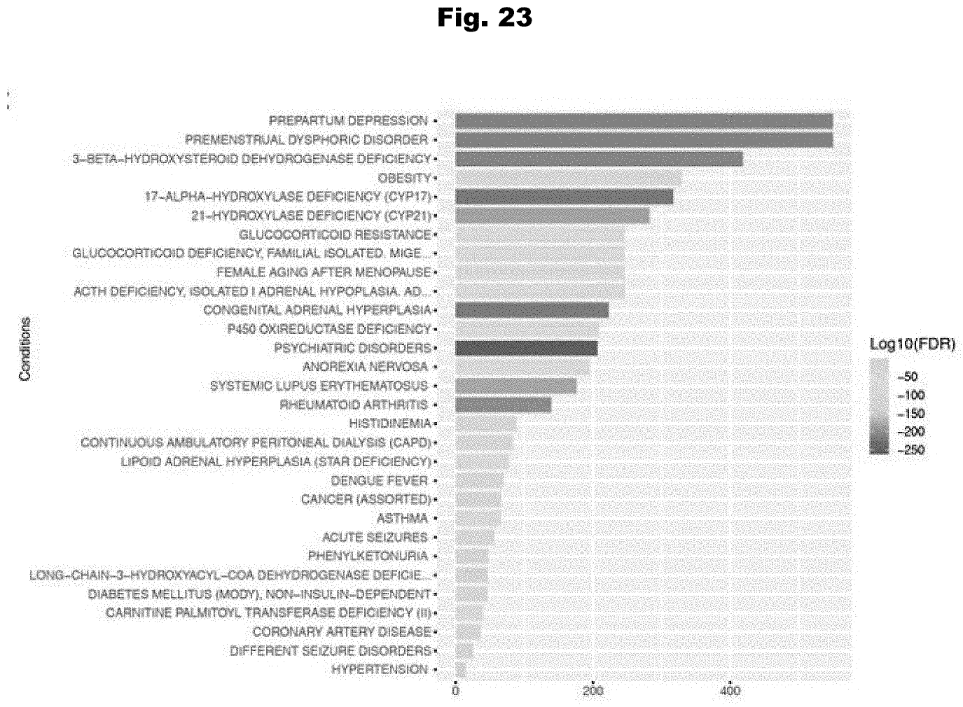

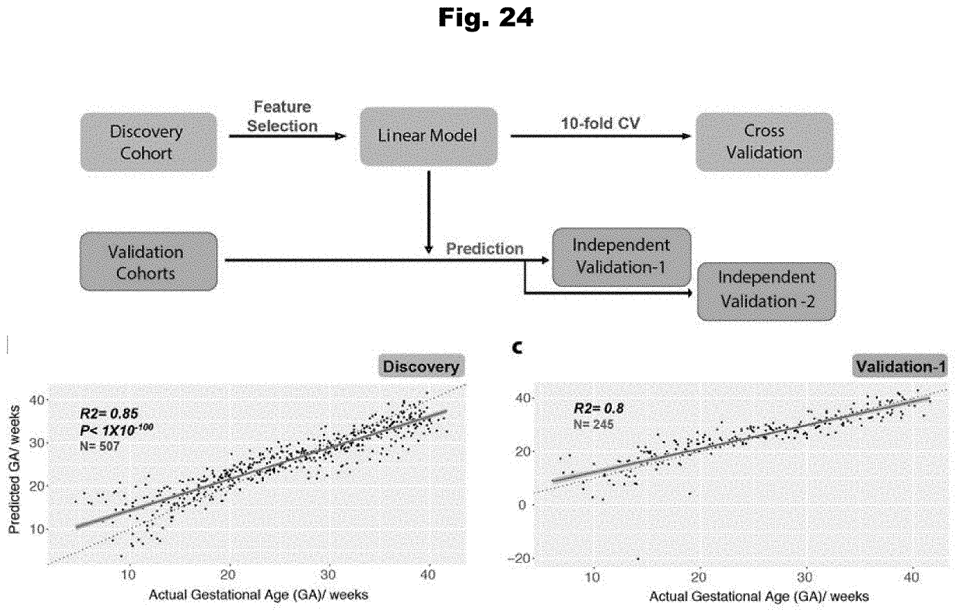

In addition to steroid pathway, dynamic pattern of metabolite changes was observed in other pathways, such as arachidonic acid metabolism pathway (FIG. 22). Specifically, an elevation of 20-HETE was observed, which links with the regulation of blood pressure and renal function. In contrast, 5-HETE showed a general decrease during pregnancy, potentially associated with its function in labor. Thus, beyond energy metabolism and hormones, a system-wide reconfiguration of the metabolome occurs in the mother during its adaptation to pregnancy. In addition, pregnancy-related metabolites are associated with medical conditions including prepartum depression and obesity (FIG. 23).

### The Metabolomic Clock of Pregnancy Revealed by Machine Learning

It was next determined whether metabolomic profiles can be used to predict gestational age for individual plasma samples. In the discovery cohort (sample N=507, subject N=21), feature selection (lasso) with all 9651 features was applied to build the linear regression model that shows optimal cross validation performance for prediction of a given phenotype in this cohort. The validation cohort data (sample N=245, subject N=9) was run through the model established in the discovery cohort, to measure the independent performance of our model (FIG. 24).

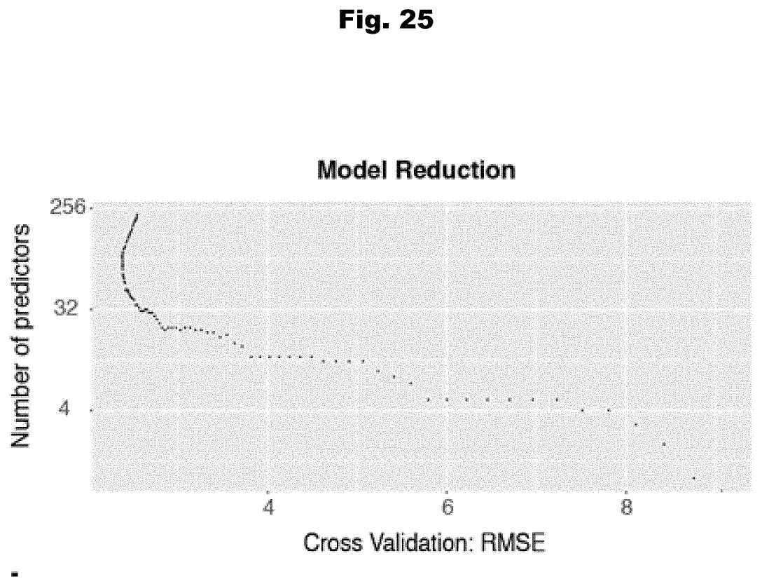

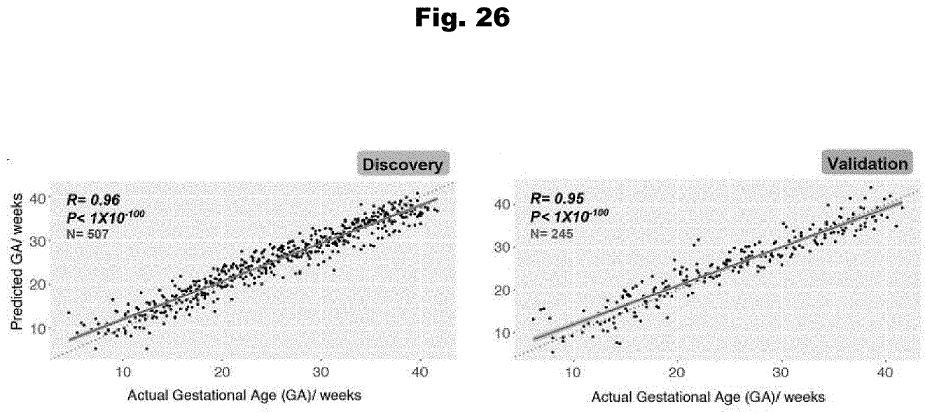

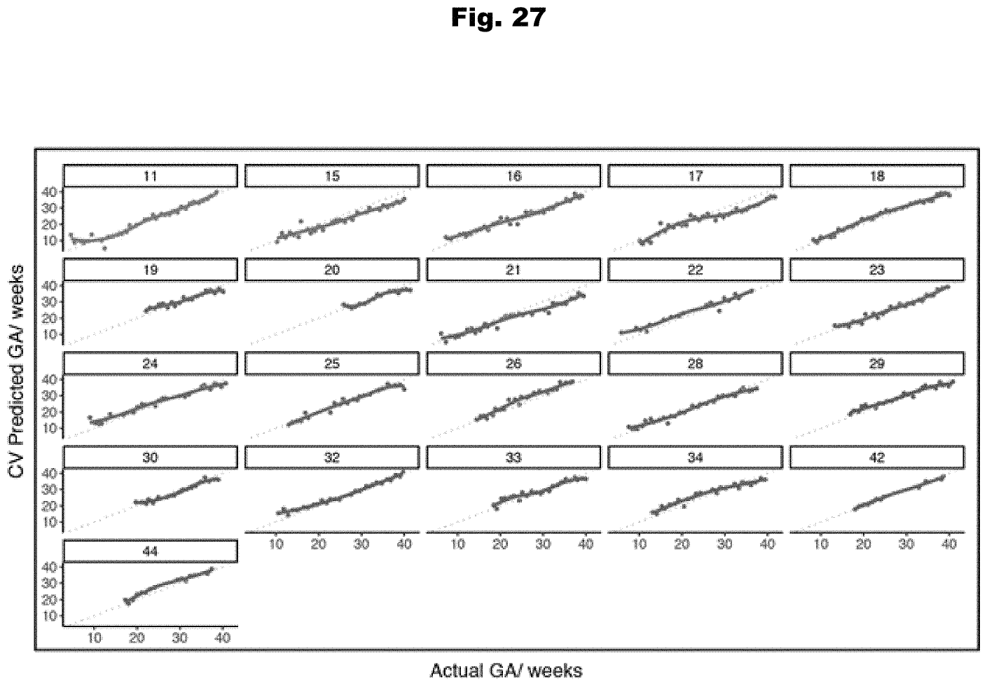

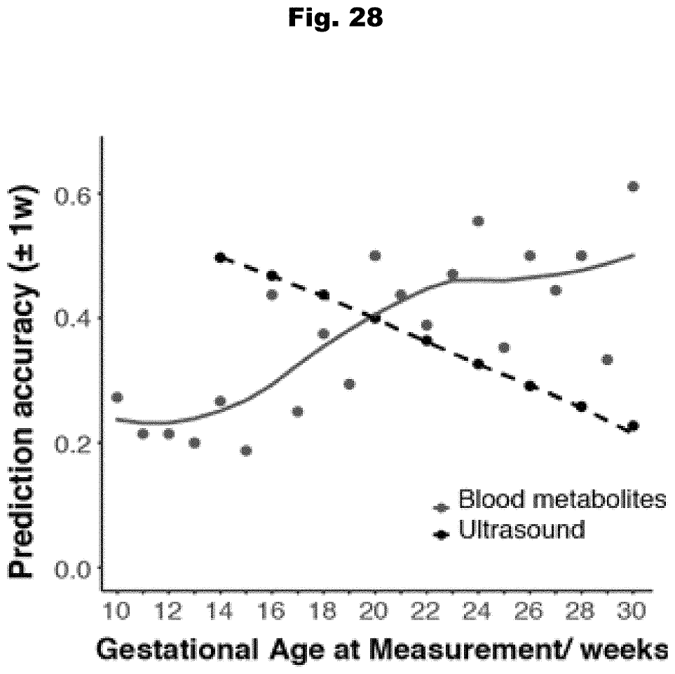

It was tested whether the metabolome alterations can quantitatively determine the GA in normal pregnant women. Feature selection in the discovery cohort yielded a linear model that included 42 metabolic features (FIG. 25, Table 3). In the cross-validation test of 507 samples in the discovery cohort, the model predicted GA weeks correlating to the actual GA weeks (determined by the first trimester ultrasound in compliance with the clinical standard-of-care) with a Pearson correlation coefficient (R) of 0.96 (P<1×10⁻¹⁰⁰, FIGS. 26 and 27). In the validation cohort, the model yielded a similar R of 0.95 (P<1×10⁻¹⁰⁰, FIG. 26). We further tested whether we can predict the timing for normal deliveries using this model for 18 women with spontaneous onset of labor. As shown in FIG. 28 (percentages of actual deliveries within +/−1 week of prediction; 18 women) although standard care ultrasound was better at predicting delivery time than the metabolic-feature model in early pregnancy, the situation reversed as pregnancy progressed, such that the metabolic prediction of delivery time was superior to ultrasound from the middle of the second trimester on to delivery. This indicates the two modes of gestational age estimation may complement each other.

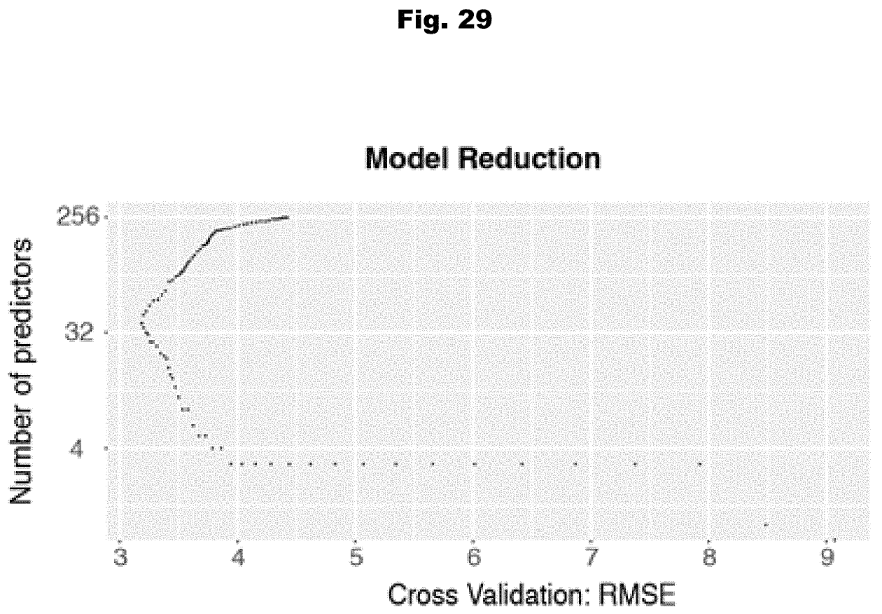

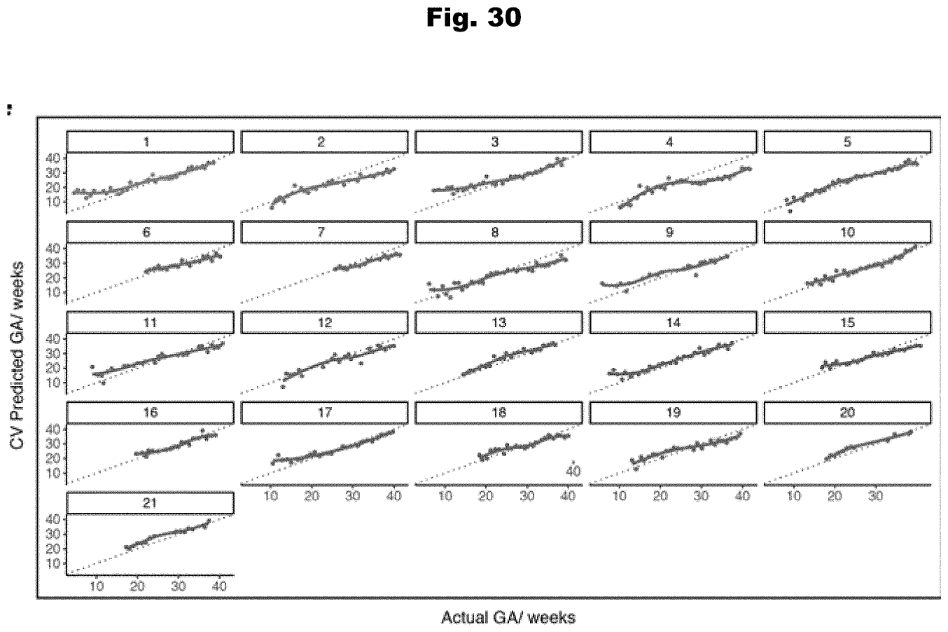

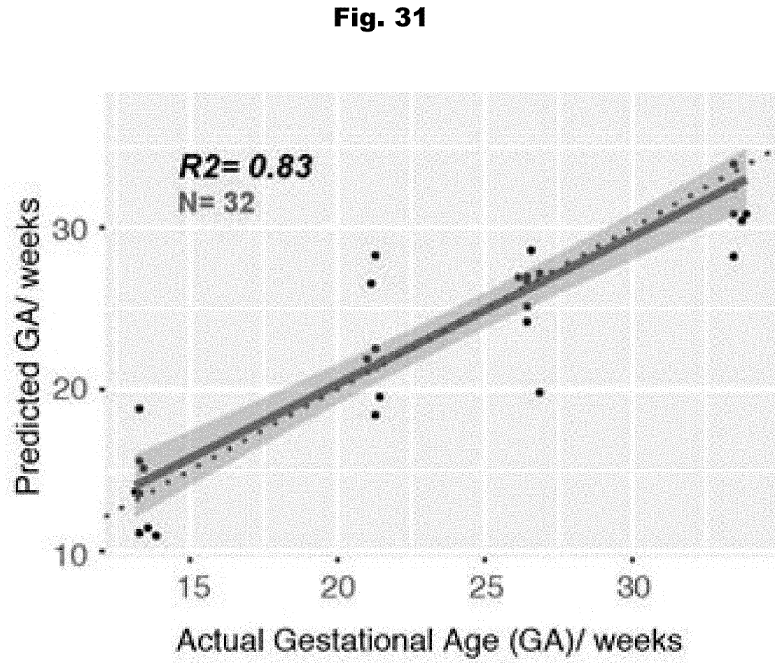

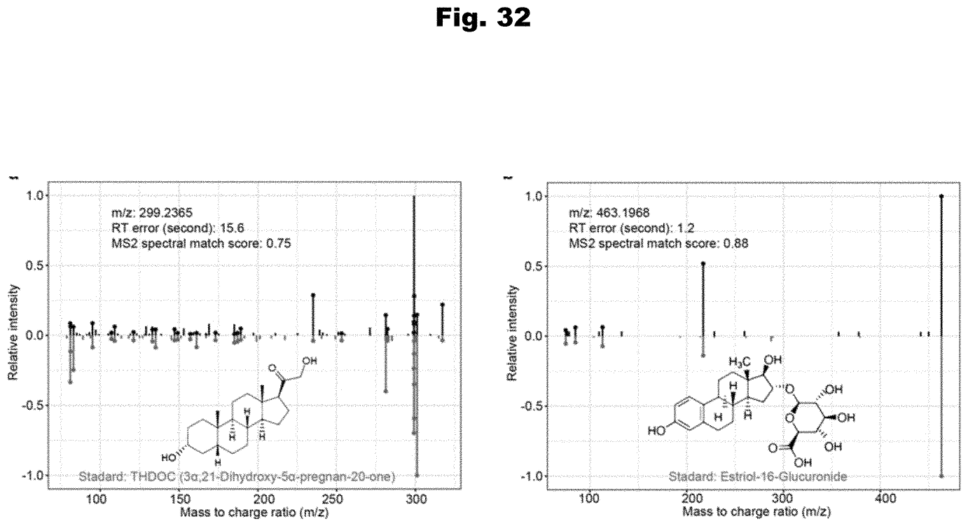

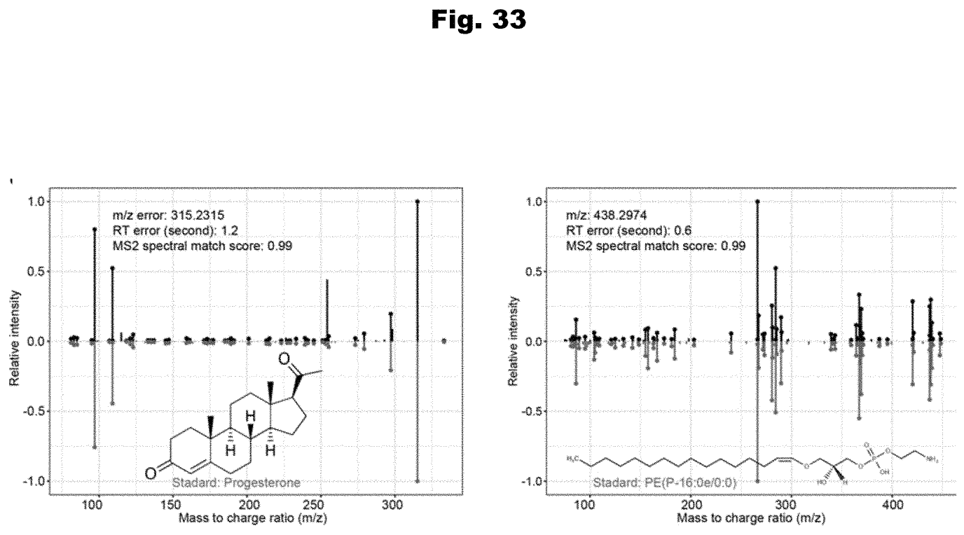

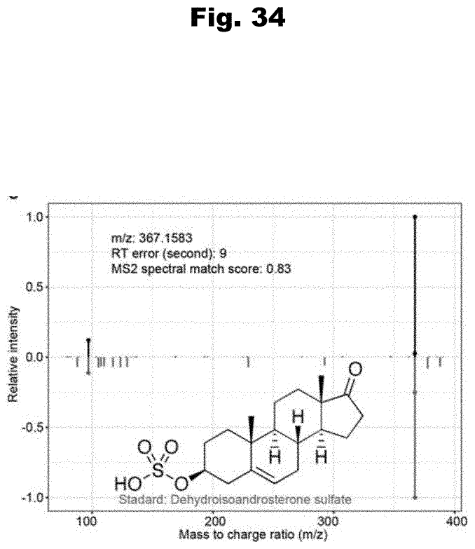

Next, it was tested whether we can use the identified metabolites in blood to quantitatively determine the gestational age (GA) in pregnant women. Feature selection using the 264 level 1 and level 2 identified HMDB compounds in the discovery cohort yielded a linear model including five compounds (FIG. 29) that together are highly predictive. In the cross-validation test in the discovery cohort, the model produced a result correlating to the actual GA (determined by the first trimester ultrasound) with a Pearson correlation coefficient (R²) of 0.85 (P<0.001, FIG. 26, FIG. 30). In the first validation cohort, the model yielded a correlation coefficient of 0.8 (P<0.001, FIG. 26). The model, including 4 steroids and one lipid (FIG. 4), was further verified in a second independent validation cohort (R²=0.83, N=32, Table 1, FIG. 31). The identifications were confirmed by their fragmentation spectra matching to MS/MS database (FIGS. 32-34). Thus, although the 42-feature model performed better, this five-compound model offers a simple alternative test, which may be preferred in a clinical setting.

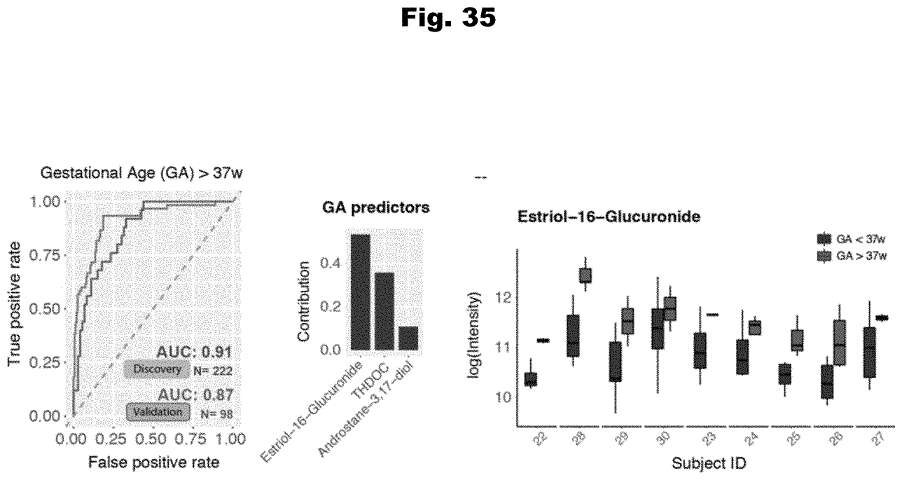

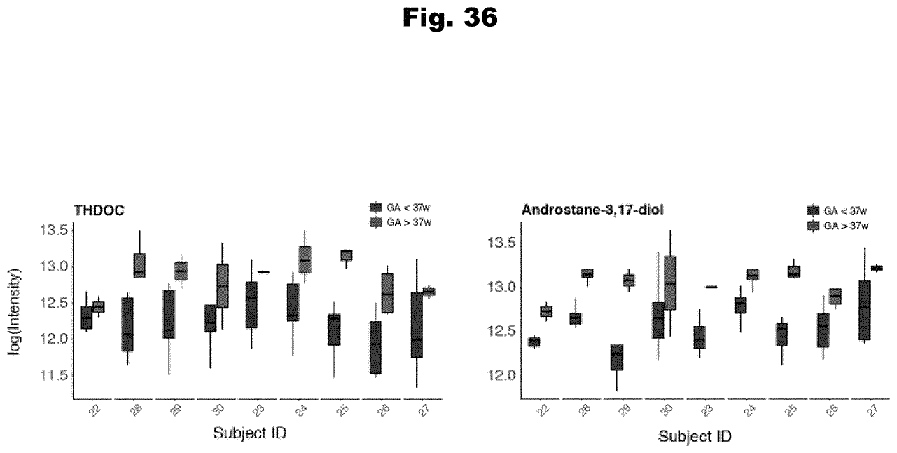

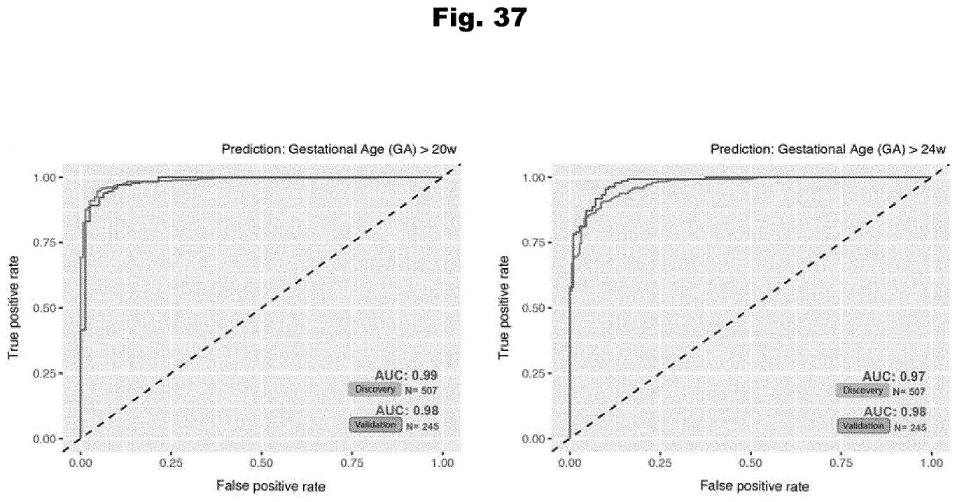

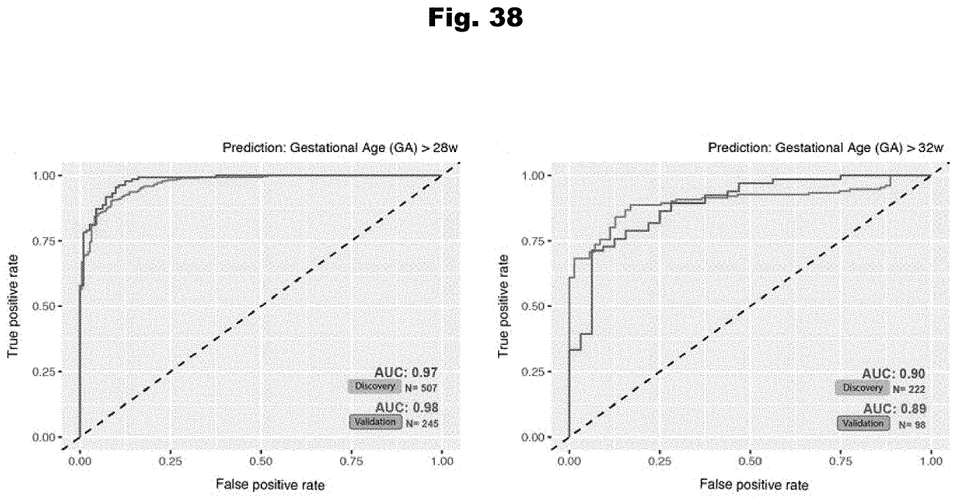

As pregnancy progresses towards term, a number of clinical classifications and decisions need to be made based on timing (e.g., <37 weeks for preterm birth). Therefore, as a proof of principle, the metabolome data was used to classify the normal pregnancy samples as before or after 20, 24, 28, 32, and 37 gestational weeks, and measured from the time of sampling to be 2, 4, and 8 weeks from delivery (FIG. 5). First, using the third trimester samples (>28 weeks of gestation) the maternal blood metabolites were assayed to distinguish the sampling GA as before or after 37 weeks. Both the discovery as well as the validation prediction yielded an AUROC over or close to 0.90 (FIG. 35). Remarkably, the prediction model contained only three metabolites, all of which showed intensity range separations for sample series derived from all but 1 to 2 validation subjects (FIGS. 35 and 36). Similarly, it was found that metabolites can also be used to distinguish pregnancy samples before or after other gestational age cutoffs, such as 20, 24, 28, and 32 gestational weeks (FIGS. 5, 37, and 38).

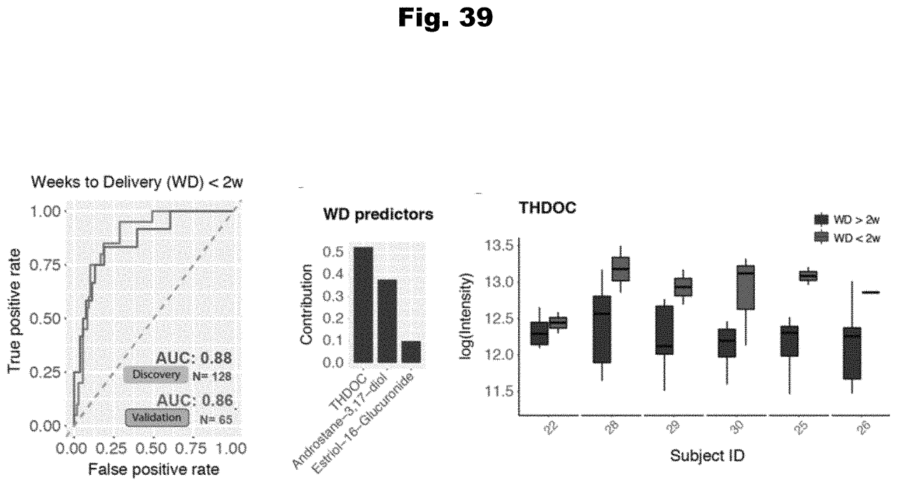

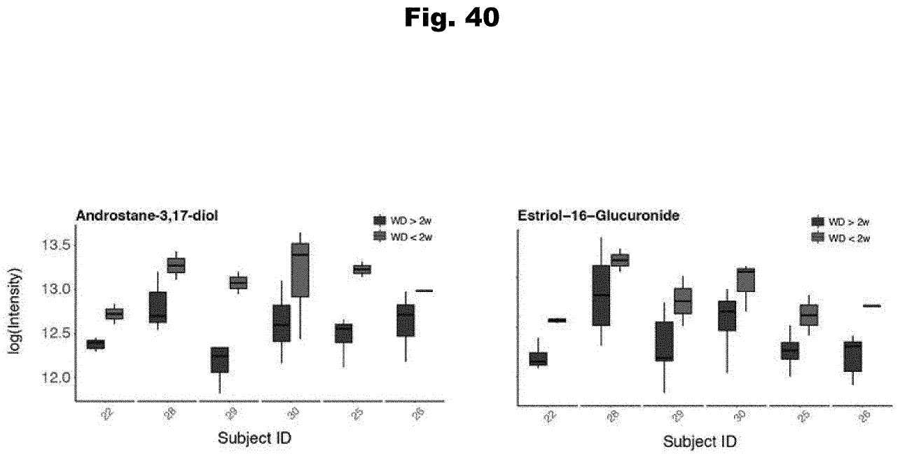

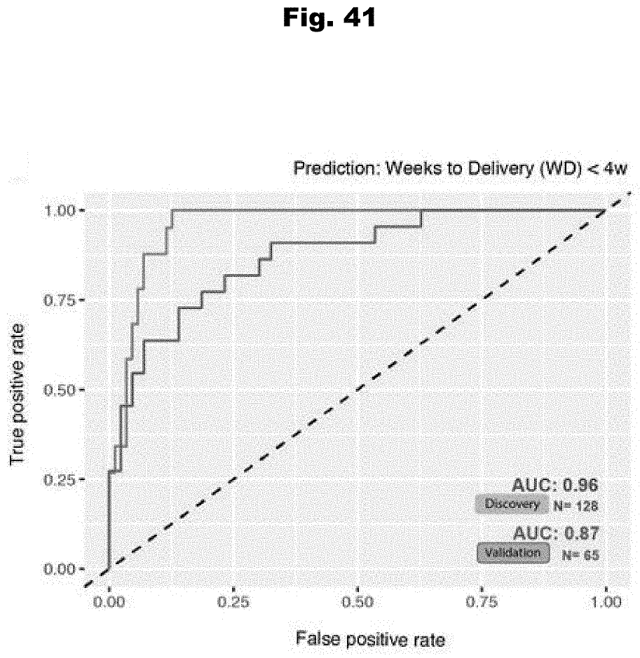

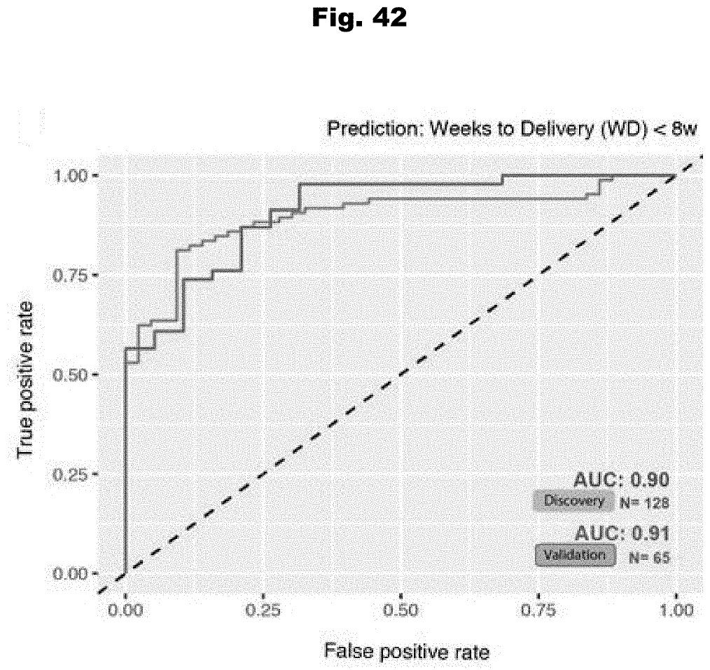

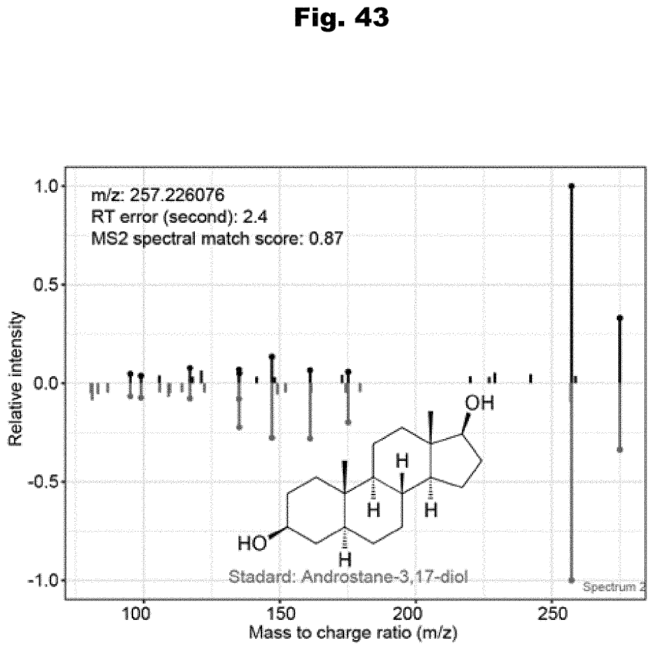

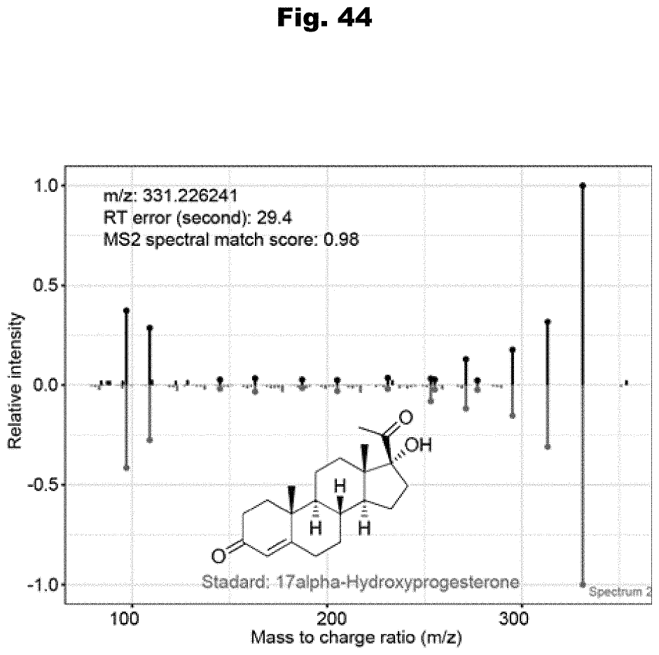

It was then tested whether the maternal blood metabolites can also predict the timing of a normal delivery event within 2 weeks (weeks to delivery, WD<2w) in the third trimester. In this test, naturally triggered delivery events were only included (subject N=18, sample N=193). The metabolome can also accurately predict the approaching of a delivery event within 2 weeks in both discovery and validation cohorts with AUROC around 0.9, using merely three metabolites (FIGS. 39 and 40). Of note, the metabolites overlapped with the metabolites that was used to predict GA<37 weeks but with different importance of contribution (FIG. 5). Similarly, metabolites can also be used to predict the timing of a normal delivery event within 4 and 8 weeks (FIGS. 5, 41, and 42). Intriguingly, the panels of metabolites are partially overlapped between models and they are all identified to be steroids except one phospholipid PE(P-16:0e/0:0) (FIGS. 5, 43, and 44). These results demonstrate that the models precisely categorizes critical pregnancy stages in normal subjects using a small number of maternal blood metabolites.

### Methods and Measurements

### Pregnancy Cohort

Pregnant women were recruited through family doctors and via advertisements (Danish IRB number H-3-2014-004). At enrollment, all women were screened to ensure that they were healthy at baseline, without chronic conditions, and without medication intake of any kind. From each woman, weekly non-fasting blood samples were collected during pregnancy and one sample was collected after pregnancy (2×9 mL EDTA tube and 1×PaxGene RNA tube).

### Plasma Sample Preparation

784 normal pregnancy samples were analyzed in 12 batches across two years. 200 μL plasma was extracted by mixing 800 μL 1:1:1 acetone:acetonitrile:methanol with internal standard mixture. The extraction mixture was vortexed and mixed for 15 min at 4° C. and incubated at −20° C. for 2 hours to allow protein precipitation. The supernatant was collected after centrifugation and evaporated to dryness under nitrogen (Biotage Turbovap). The dry extracts were reconstituted with 200 μL 1:1 methanol:water before analysis.

Metabolic extracts were analyzed by reversed-phase liquid chromatographic (RPLC) MS, in both positive and negative ionization modes. RPLC separation was performed using a Zorbax SBaq column 2.1×50 mm, 1.8 μm (Agilent Technologies). The mobile phase solvents consisted of 0.06% acetic acid in water (phase A) and 0.06% acetic acid in methanol (phase B). A Thermo Q Exactive plus and Q Exactive mass spectrometers were operated in full MS scan mode for data acquisition. Pooled samples from pregnant women and within each batch were used for quality control. MS/MS data were acquired with different collision energies (NCE 25 and 50).

Plasma was prepared from whole blood treated with anti-clot EDTA and aliquoted and stored at ×80° C. 200 μL Plasma was treated with four volumes (800 μL) of an acetone:acetonitrile:methanol (1:1:1, v/v) solvent mixture with internal standards, mixed for 15 min at 4° C. and incubated for 2 h at −20° C. to allow protein precipitation. The supernatant was collected after centrifugation at 10,000 rpm for 10 min at 4° C. and evaporated under nitrogen to dryness. The dry extracts were reconstituted with 200 μL 50% methanol before analysis. A quality control sample (QC) was generated by pooling up all the plasma samples from 10 women and injected between every 10-15 sample injections to monitor the consistence of the retention time and the signal intensity. The QC sample was also diluted by 2, 4 and 8 times to determine the linear dilution effect of metabolic features.

### Bioinformatics and Statistics

Acquired data were processed using an analysis pipeline written in R. Metabolic features were extracted with a unique mass/charge ratio and retention time, then aligned and quantified with the Progenesis QI software (Nonlinear Dynamics). Linear normalization was applied to adjust the signal variations along the running process. In total, 9,651 features were included in the final analysis. Metabolite identification was performed by matching the accurate masses (m/z, +/−5 ppm) and retention time against in-house library, and further by matching the accurate masses and MS/MS spectra against public database, including HMDB, MoNA, MassBank, METLIN and MassBank. Then the MS/MS spectra match were manually checked to confirm the identifications, which were considered as the level 2 identification according to MSI. The metabolic features that have no match in the databases were further analyzed by MetDNA. Finally, he major machine-learning model predictors were confirmed with chemical standards by matching the accurate masses (5 ppm), retention time (30 seconds), and MS/MS spectra.

Section 1: Metabolomic features were extracted with a unique mass/charge ratio and retention time, then aligned and quantified with the Progenesis QI software (Nonlinear Dynamics, Durham, N.C., USA). Acquired data were processed using an analysis pipeline written in R. Progenesis Q output was then processed by removing all metabolites which were quantified in less than 30% of the samples or showed high signal to noise (median signal less than double the median signal in blank measurements). Data was globally normalized by applying a median correction for each run to correct for sample amount variation. Analyte levels were further normalized by fitting a linear regression to each batch to correct for linear changes in sensitivity and analyte degradation over time. A median correction was applied to normalize data between batches. In total, 9,651 features were included in the final analysis.

Section 2: PCA Analysis—Principal component analysis (PCA) was applied to examine the overall distribution of the sample data (with all 9651 features) as well as to check the run quality. The gestational ages (based on ultrasound measurements) were super-imposed to facilitate the analysis. During the analysis, vast majority of the samples were separated by pre- and postpartum in PCA space defined by two components which explained the largest variations (PC1 and 2, FIG. 6), while two samples of a same subject (last two in her collection, before and after child birth) displayed irregular behavior in PCA and unsupervised clustering analysis. The 2 samples were treated as outliers and excluded for further analysis.

Section 3: Identify Significantly Altered Features/Compounds—A statistical method specialized for multi-testing, SAM (Significance Analysis of Microarrays) was applied to identify metabolic features/compounds altered significantly in metabolome-wide analysis. For all SAM analyses, distribution-independent ranking tests (based on the Wilcoxon test) were used to ascertain significance (false discovery rate, FDR<0.05). The adjusted GAs were included in a number of plots to present the changes of metabolites among individuals, which were calculated by scaling all delivery event timing to 40 weeks.

Section 4: Machine Learning for Pregnancy Timing—Two cohorts of data collected and run at different years but from the same center were used to establish discovery (Subject N=21, sample N=507) and validation (Subject N=9, sample N=245) datasets. Lasso (R package: glmnet) was applied in the discovery dataset to 9651 features to build the linear regression model to predict GA. A 10-fold cross validation was performed to choose optimal lambda (penalty for the number of features). The model performance was evaluated using two different methods: 1) During the cross validation in the discovery dataset, for each fold, the predictions under the optimized lambda were recorded and pooled together. 2) The model was built using the optimized lambda and the full discovery datasets. This model was applied to the validation cohort for prediction and verification. A linear fitting from the two above evaluations were performed, between the predicted value and the actual values, with Pearson correlation coefficient (R) reported.

It was then tested whether the predicted GA was able to predict the delivery timing in the form of Δ (40-observed GA). The prediction from cross-validation in the discovery dataset and the independent validation was pooled together. Only the 18 women (out of 30) with natural labor onset were chosen, excluding subjects with events such as induction before labor onset and scheduled C-section (induction by oxytocin/membrane strip after the onset is allowed). In clinic, the prenatal visits are often recommended in a timed series (e.g., once every 2 weeks for week 28 to 36). To mimic the clinical setting, for each woman, a rolling window of 8 weeks was utilized, which were divided into 4×2 week sub-windows. In each 2 week window, the first sample was used to perform the GA prediction. No more than one missing test was allowed for these 4-test series. The medians of the predicted values from the 4-test series were taken to calculate the Δ (40-observed GA). The accuracy was calculated as the percentage of women (out of the 18) delivered within +/−1 week of the predicted A (40-observed GA) value. For a longitudinal comparison between the accuracies of blood metabolite prediction and ultrasound estimation, general ultrasound accuracy from 14-week to 30-week were calculated based on the published data (according to LMP), with the slop scaled according to the first trimester ultrasound accuracy in the present study (0.5).

For >28 weeks samples (the third trimester), we also started with 9651 features and used a similar discovery and validation pipeline described for GA prediction (above) to build logistic regression models predicting the categorical labels of GA>37 weeks or delivery within 2 weeks. For the prediction on delivery within 2 weeks, only the 18 women (out of 30) with natural labor onset were included, excluding subjects with induction before labor onset and scheduled C-section (induction by oxytocin/membrane strip after the onset is allowed).

Section 5: Metabolic Features Identification—Metabolite identification was performed using two-step approach. First, the in-house metabolite library was used to identify compounds, containing chemical standards and manually curated compound list based on accurate mass and spectral pattern. Second, further metabolites were putatively identified based on accurate mass, isotope pattern and fragmentation spectra matching using the MS/MS databases of METLIN3, NIST, CCS (Waters), Lipidblast4 (precursor tolerance: 5 ppm; isotope similarity>95). The Pearson correlation was examined for each pregnancy-related compound identified, using the intensities of metabolites across all samples.

Section 6: Pathway Analysis—The compound identification (standards, MS2 and in-silico m/z only) were pooled together. Each metabolic feature was allowed only to match to a single compound to avoid over-representation. When in the rare cases, a given metabolic feature was matched differently between different matching methods, the matching was choosen based on the identification level: standards>MS2>in-silico m/z only.

MetaboAnalystR was utilized to perform the metabolite set enrichment analysis (MSEA) as well as metabolic pathway analysis (MetPA) on all identified metabolites. To quantify the pathway activity, the intensities of all identified metabolites was averaged for each pathway and plotted on the heatmap (FIG. 35). The pathway activity before 14 weeks were averaged across all available samples and subtracted from all later time-points. The statistical significance of the alteration of a pathway's activity across pregnancy was evaluated by global test.

### Mass Spectroscopy Acquisition

MS acquisition was performed on an Q Exactive Hybrid Quadrupole-Orbitrap mass spectrometer (Thermo Scientific, San Jose, Calif., USA) cooperating in both the positive and negative ion mode (acquisition from m/z 500 to 2,000) using a resolution set at 30,000 (at m/z 400). The MS2 spectrum of the QC sample was acquired under different fragmentation energy (25 NCE and 50 NCE) of the top 10 parent ions. The resulting mass spectra were exported into Progenesis QI Software (Nonlinear Dynamics, Durham, N.C., USA) for further processing.

### Chromatographic Conditions

Zorbax SB columns (2.1×50 mm, 1.8 Micron, 600 Bar) were purchased from Agilent Technologies (Santa Clara, Calif., USA). Mobile phases for RPLC consisted of 0.06% acetic acid in water (phase A) and MeOH containing 0.06% acetic acid (phase B). Metabolites were eluted from the column at a flow rate of 0.6 mL/min, leading to a backpressure of 220-280 bar at 99% phase A. A linear 1-80% phase B gradient was applied over 9-10 min. The oven temperature was set to 60° C. and the sample injection volume was 5 μL.

### Example 2: Protein Dynamics and Human Pregnancy

During pregnancy, numerous molecules undergo systematic changes to interactively and coordinately advance progression and outcome. Measuring the molecular dynamics throughout pregnancy and the postpartum period likely provides insights regarding the biological processes that occur during pregnancy, and can enable monitoring of gestational progress, including identification of protein biomarkers associated with early maladaptive pregnancy. In some embodiments, a diagnostic or prognostic detection provides an actionable determination, which can be utilized to further assess and/or treat an individual. Various embodiments utilize biological fluids for diagnostics, such as plasma, which are generally considered to be rich and minimally invasive sources for monitoring dynamics of different types of molecules.

The proteome both directs and reflects physiological processes. The large variation in the abundance of plasma proteins, which spans at least 14 orders of magnitude, presents a significant technical challenge for detecting the full spectrum of proteins, particularly those in low abundance. To date, plasma protein studies in pregnancy have been limited to a handful of informative proteins. For instance, pregnancy-associated plasma protein A (PAPP-A) has shown clinical association with the development of preeclampsia and with stillbirth. Additional pregnancy studies of the plasma proteome using Somalogic and Luminex technologies identified numerous predictive proteins corresponding to gestational trimester and revealed maternal immunological adaptations over the course of gestation. The largest such study was analyzed with Somalogic 1,310-plex and Luminex 62-plex protein assays (for more on the study, see R. Romero, et al., American journal of obstetrics and gynecology 217, e61-67 (2017); and N. Aghaeepour, et al., Science immunology 2, (2017), the disclosures of which are incorporated herein by reference). Romero and colleagues used 200 samples collected in individual trimesters to identify a putative immune clock and Aghearrpour and colleagues used 81 samples found molecules correlating with gestational week.

In the present example, of the Danish cohort of pregnant women was utilized. Plasma was sampled weekly during pregnancy and once within 6 weeks after parturition. For this particular study the weekly sampled plasma specimens were extracted during the first trimester and monthly samples were extracted during the remaining pregnancy. This dense sampling provides an opportunity to observe high-resolution proteomic dynamics in plasma across pregnancy and postpartum. A highly robust, sensitive multiplex proximity extension assay was used to simultaneously analyze a diverse set of low- and high-abundance plasma proteins. Using this assay, the levels of 363 proteins across pregnant gestation in a total of 261 samples were measured. Furthermore, to study labor in greater detail 436 proteins were measured in the samples collected within a week of labor (n=30) and postpartum (n=29). In this study first-trimester spontaneous abortion samples were collected weekly and their first-trimester controls, these 436 proteins were detected in samples from these women having undergone spontaneous abortions (n=7, a total of 20 samples collected weekly), and statistically compared to levels in the control group of normal pregnancies (n=21, total 65 samples collected weekly in the first trimester) (Table F).

### Concordant Dynamics of the Plasma Proteome in Human Pregnancy

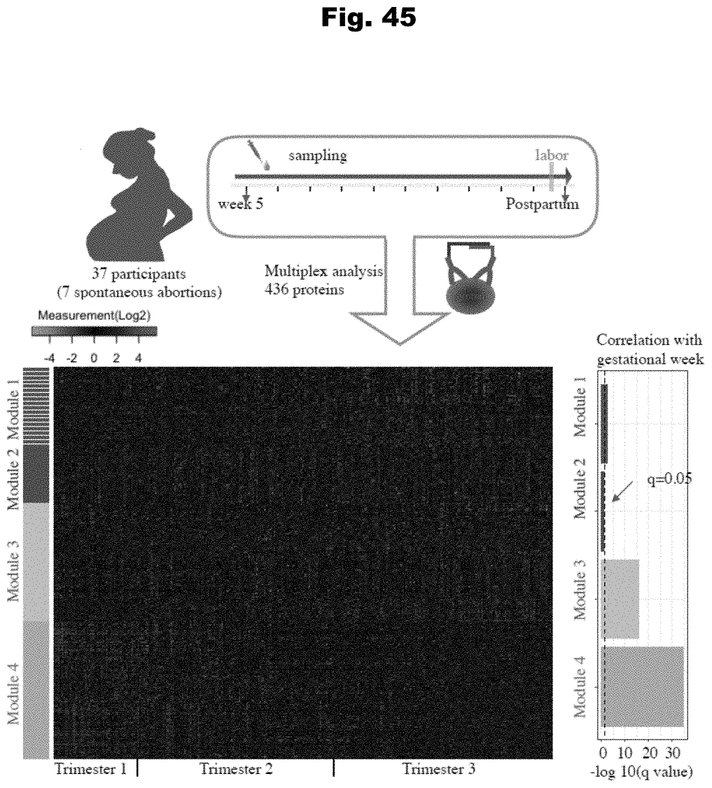

To understand the dynamic changes of protein levels from early pregnancy to parturition, the levels of 363 proteins in human plasma samples drawn monthly from 30 women during pregnancy were analyzed (FIG. 45). Protein levels were analyzed using highly sensitive proximity extension assays capable of detecting proteins that vary in concentration by over ten orders of magnitude. In multiplex proximity extension assays, pairs of oligonucleotide-conjugated antibodies are used to target each of 92 proteins and 4 controls in 1 μl plasma samples and the DNA extension of the pair of oligonucleotides that form upon target recognition are quantified by quantitative PCR. The assay was applied to the 261 pregnant samples and the data collected at each time point was normalized by quantile and Combat normalization to remove batch effects.

The protein levels (363 proteins in 261 pregnant samples) were grouped into discrete co-expression patterns using two different approaches: weighted correlation network analysis (WGCNA) and Fuzzy c means clustering. In the WGCNA approach, modules were identified with a topological overlap dissimilarity score via adjacency scores, followed by hierarchical clustering. Adjacency score in WGCNA is defined as the correlational strength between changes of expression levels of individual proteins in plasma across all gestational samples. As shown in the clustering analysis, the expression levels of all the proteins were highly correlated and their dynamics were concordant across the pregnancies (FIG. 45, see heat map). The significance of the correlations between individual four modules of highly correlated proteins and gestational week was calculated (Table 3), and three modules (modules 1, 2, and 4) proved significantly associated with gestational week (q<0.05; FIG. 45, see graph).

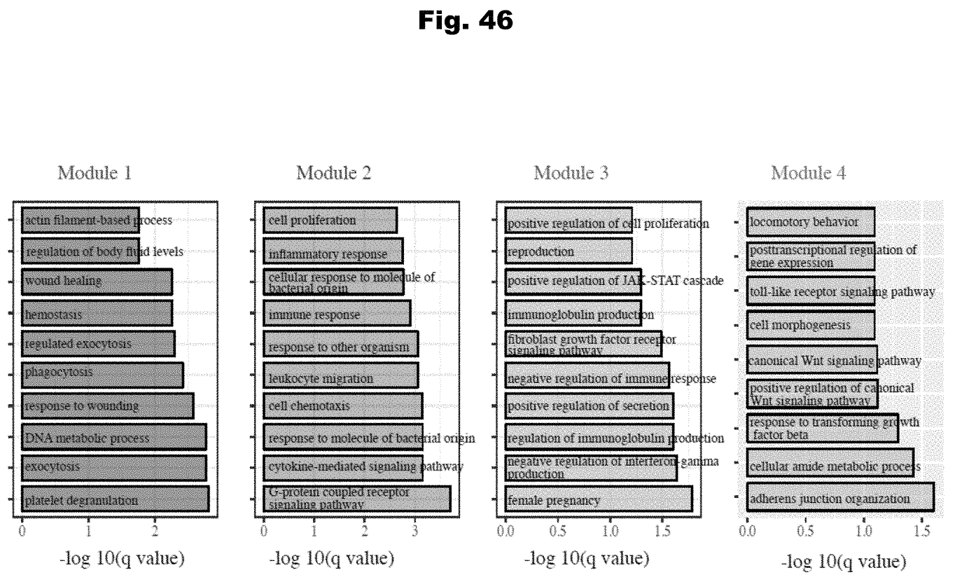

Enrichment of gene ontology (GO) terms were investigated for proteins within individual modules, and the enriched GO terms revealed a range of enriched biological processes (FIG. 46). For instance, expression levels of proteins in module 3 gradually decreased as pregnancy progressed, and this module included enriched GO terms for: biological processes functionally associated with pregnancy, negative regulation of immune responses, regulation of the JAK-STAT cascade and reproduction. Module 1 included plasma proteins that were highly expressed but gradually decreased during pregnancy, with their GO terms reflecting enrichment of DNA metabolic processes and platelet degranulation during pregnancy. Module 4 was comprised of plasma proteins that were weakly expressed in early pregnancy and slowly increased as pregnancy advanced. Proteins in this module are involved in the toll-like receptor signaling pathway, which plays key roles in the innate immune response, as well as in Wnt signaling pathway processes. Interestingly, module 2 was not significantly correlated with gestational weeks during pregnancy. Uniform elevated expression across pregnancy of proteins involved in cell proliferation, immune response, and cytokine-mediated signaling pathways was observed.

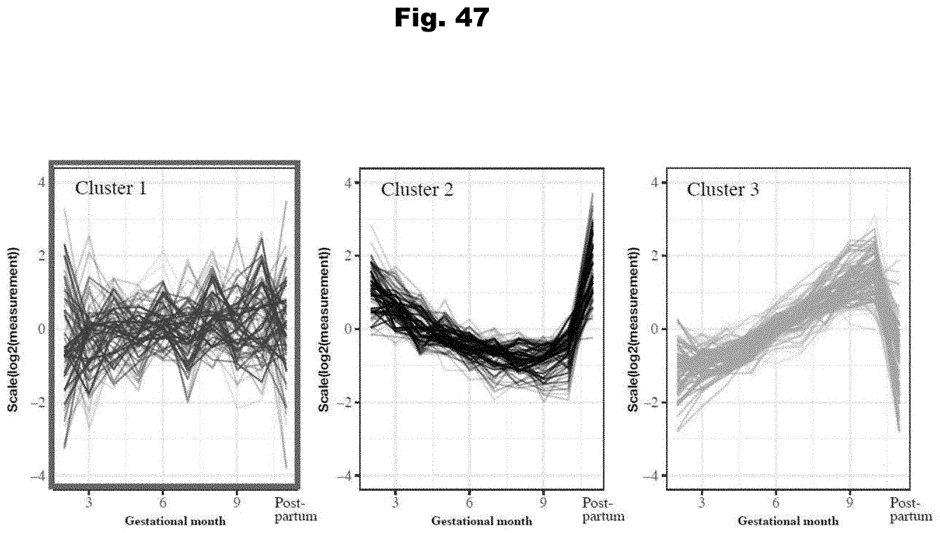

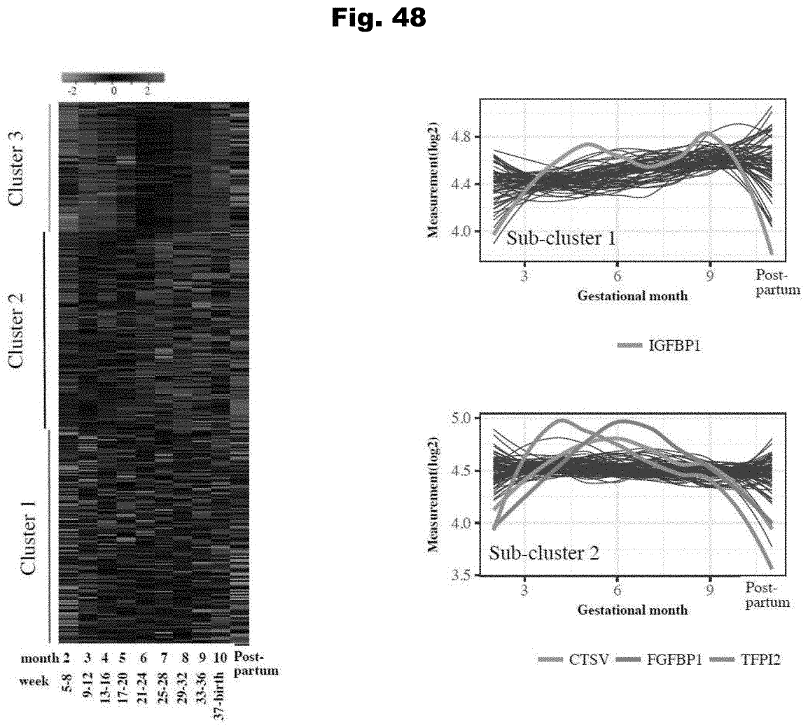

As a second approach, monthly changes of the 363 protein levels across pregnancy and the postpartum was examined using Fuzzy C-means (in total 290 samples). The optimal number of three clusters was determined using the bootstrap approach, with proteins grouped in individual patterns based on changes of their levels and co-expression (FIGS. 47 and 48, and Table 4). For instance, the levels of the C-X-C motif chemokine 13 (CXCL13), myeloperoxidase (MPO) and C-C motif chemokine 23 (CCL23) in the cluster 2 decreased across pregnancy but increased immediately after labor, whereas levels of von Willebrand factor (vWF), the C—C motif chemokine 28 (CCL28), Trefoil factor 3 (TFF3) and urokinase-type plasminogen activator (uPA) in cluster 3 increased during gestation but decreased following the postpartum. Monthly measures of proteins in cluster 1 revealed two distinct groups. In one group levels of IGFBP1 (FIG. 48) slowly increased during the course of pregnancy, remaining higher compared to its level of the postpartum, with two peaks at early second trimester and prior to labor. In the second group, levels of cathepsin V (CTSV), fibroblast growth factor-binding protein 1 (FGFBP1) and tissue factor pathway inhibitor-2 (TFPI2) peaked in the early or late second trimester.

### Protein Dynamics in Plasma Robustly Predict Chronology of Human Pregnancy

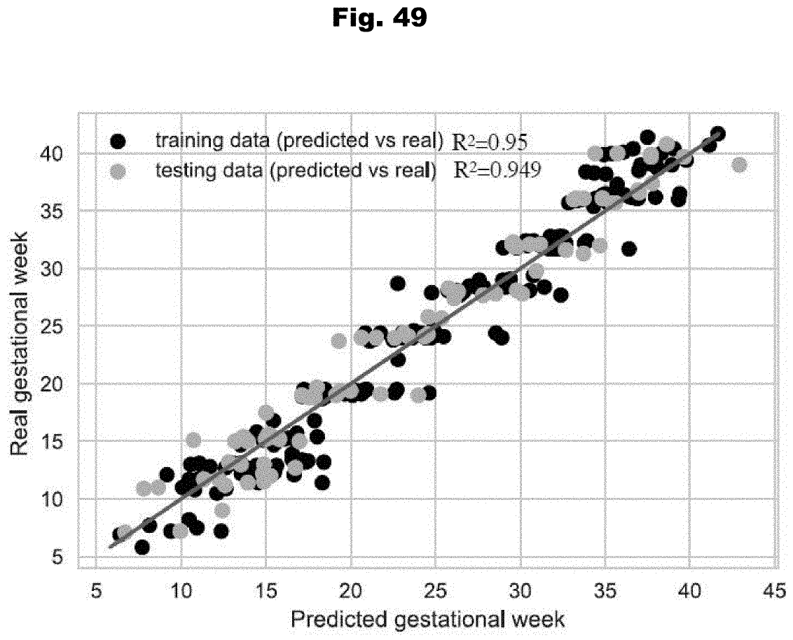

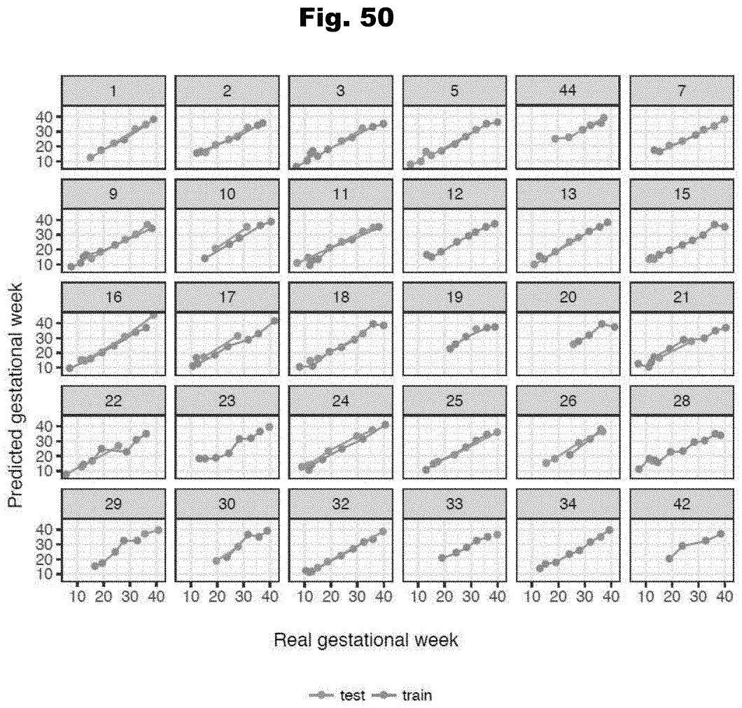

After characterizing molecular changes and identifying molecular patterns across pregnancy, the highly correlated plasma proteome data was utilized to predict gestational week of samples collected during pregnancy. The elastic net (EN) with regularization method was utilized to perform analysis due to the fact the data set is inter-correlated. The dataset was randomly divided into training and testing datasets (ratio of training dataset/testing dataset=70%/30%), and the EN regularized algorithm was applied, with 5-fold cross validation, to infer a regression module on the training dataset. The regression module was then applied to the testing dataset to evaluate its performance. The EN-based algorithm identified a predictive EN module for the training data (n=180), which drives the strong association between predicted gestational week and observed gestational week (R2=0.95, FIG. 49; root mean squared log error (RMSLE)=0.109). The EN module was then applied to the testing dataset (n=78), where it reliably predicted gestational week of samples (R2=0.949, FIG. 49; RMSLE=0.116). Such robust prediction was revealed at the individual level (FIG. 50); each of the multiple plots demonstrates the performance of the EN model on both the training and testing datasets derived from the same individuals.

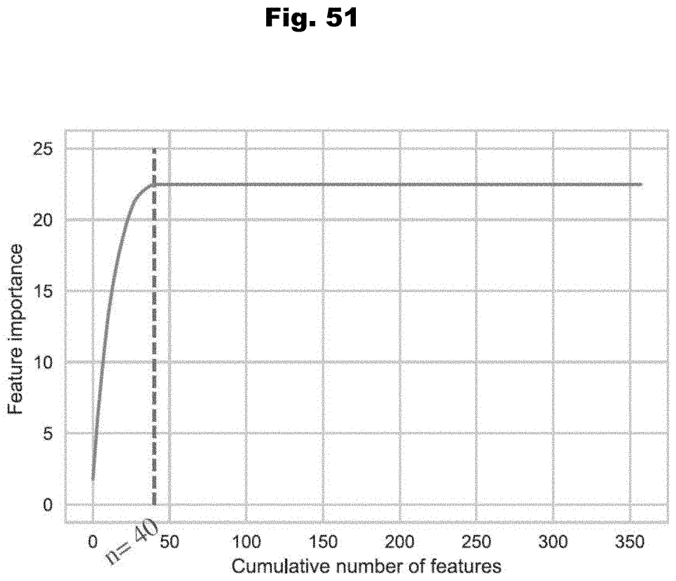

The EN model was made possible by attributing positive or negative coefficients to a group of essential proteins, termed features. For this analysis a panel of proteins (n=40, FIGS. 6 and 51) was selected and together produced the predictive model. These 40 essential proteins are involved in signaling response to stimulus and their regulation, including BDNFINT-3 growth factors receptors NTRK2, NTRK3, CCL28, IL2RA, CD200R1, uPA (urokinase-type plasminogen activator), uPAR (urokinase plasminogen activator surface receptor), CCL28, MCP/CCL8, ESM1 (endothelial cell-specific molecule 1), FcRL2 (Fc receptor like protein 2), and LAIR2 (leukocyte-associated immunoglobulin like receptor 2).

### Levels of Proteins Encoded Across Human Genome are Influenced by Labor

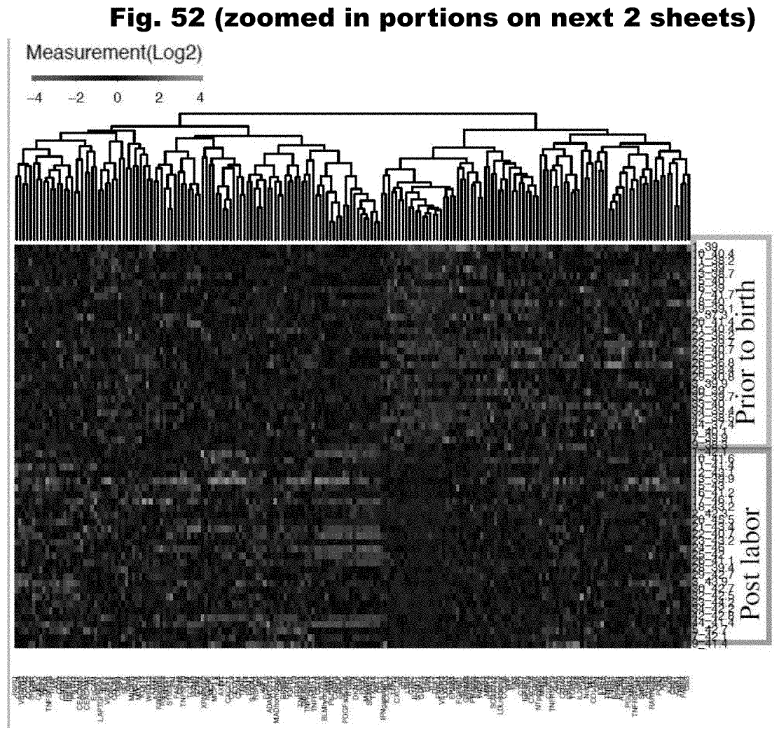

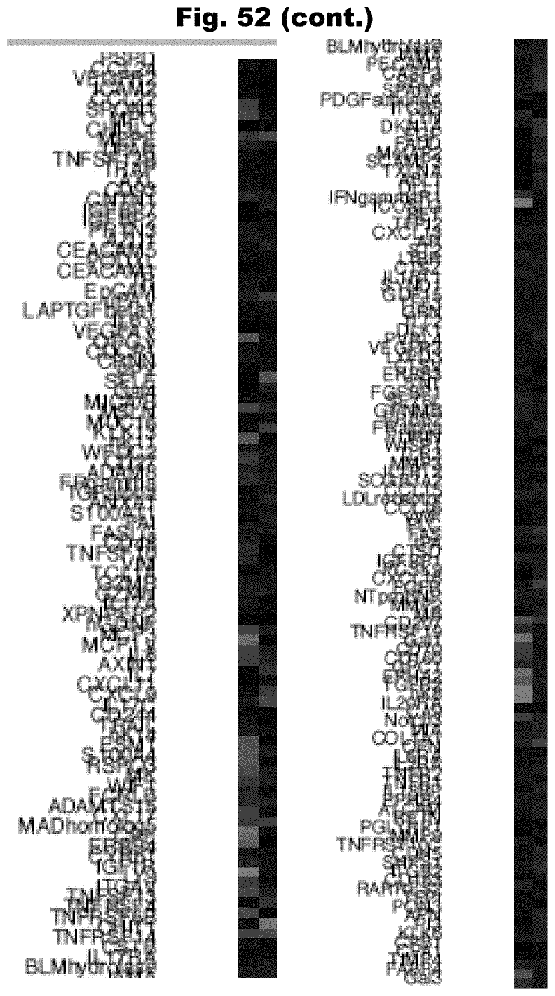

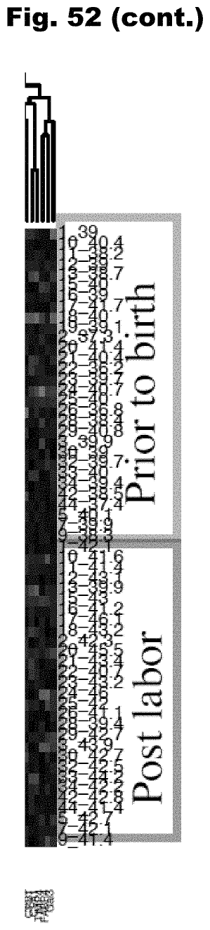

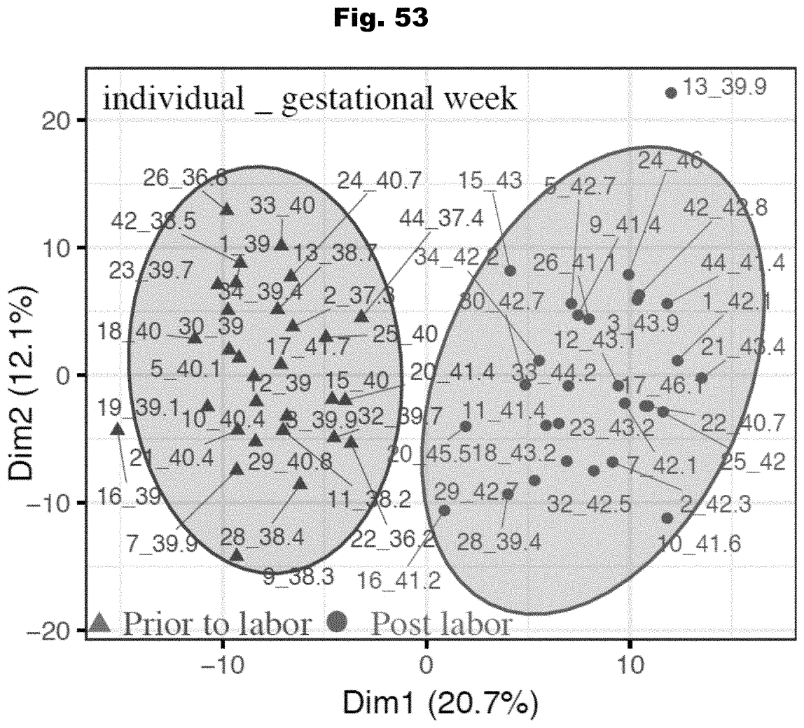

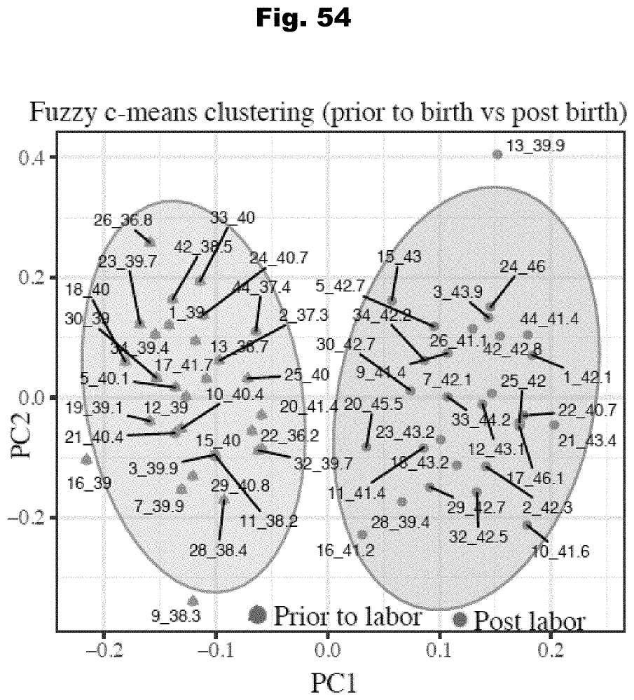

It was also sought to identify significantly changed proteins associated with labor. Samples (n=30) collected within a week before labor were compared with samples (n=29) collected at the first postpartum visit that usually occurred within 6 weeks following labor. Of the 436 total proteins, levels of 244 proteins were altered significantly (q<0.05) before and after parturition (Table 5). Since many proteins were co-expressed and interdependent, an attempt to identify groups with similar expression profiles was performed. Two methods were used: hierarchical and principal component analysis. Unsupervised hierarchical clustering revealed two major clusters of all proteins (FIG. 52), and the first dimension (PC1) of the principal component analysis (PCA) of all proteins clearly separated the samples prior to labor from the postpartum samples, whereas the second dimension (PC2) captured individual variation present in each of the groups (FIG. 53), agreeing with the result of fuzzy C-means clustering (FIG. 54).

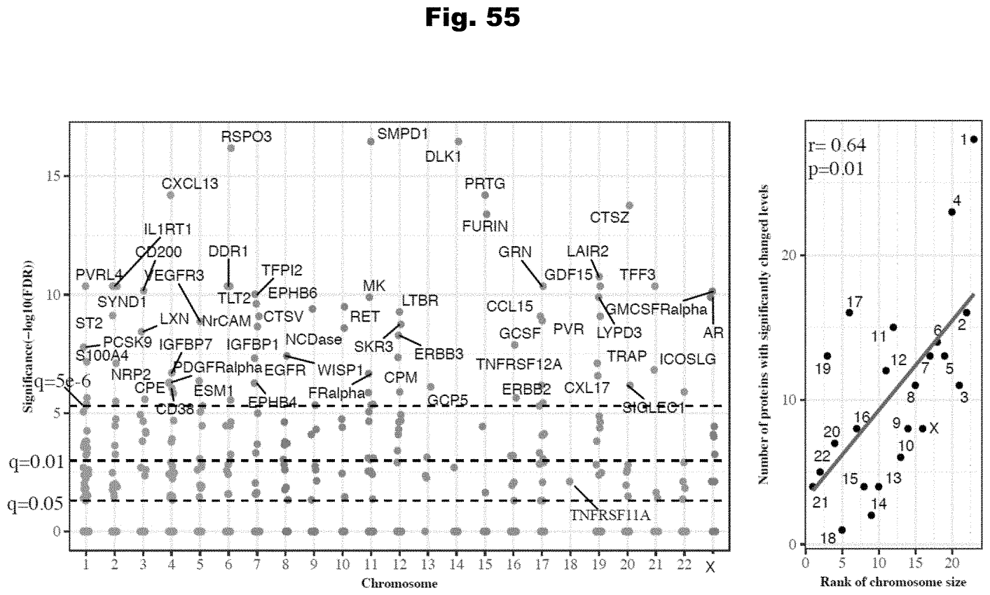

The genomic location of genes encoding all 436 proteins were examined and it was found that all 23 chromosomes were involved in encoding the proteins whose levels changed significantly before and after parturition (FIG. 55). For instance levels of CXCL13 from chromosome 4, IL1RT1 from chromosome 2 and GDF15 from chromosome 20 all changed significantly (q<0.01) before and after parturition. The number of significantly changed proteins from individual chromosome correlated with the sizes of individual chromosomes (r=0.64 and p=0.01, FIG. 55).

### Plasma Proteins Involved in Spontaneous Abortion

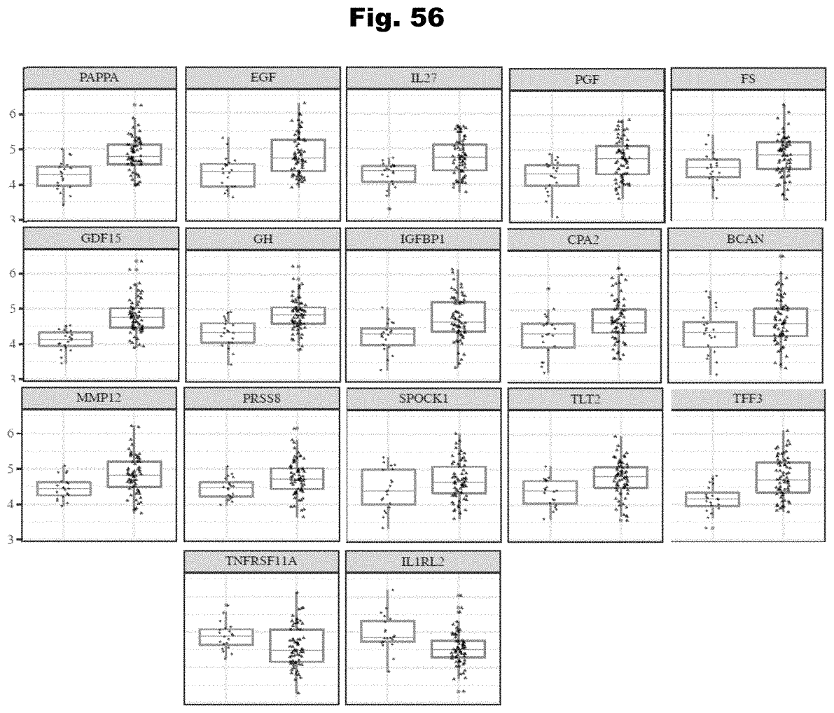

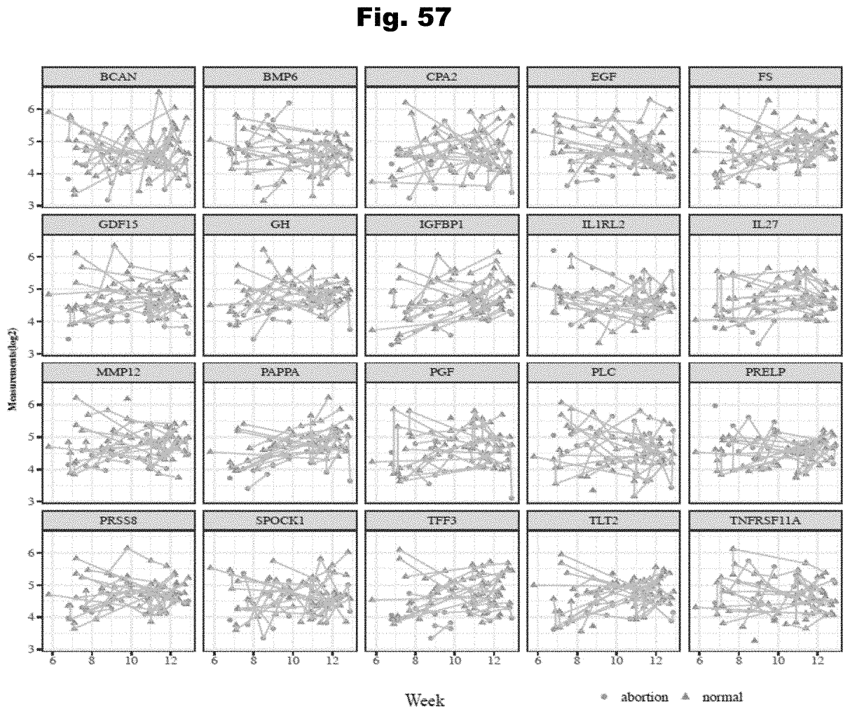

Two groups of samples, obtained from 7 women with spontaneous abortions in the first trimester and first trimester samples from 21 women with normal pregnancies (full-term singleton), were analyzed with respect to levels of 436 plasma proteins. Twenty proteins had levels that differed significantly between the abortion and control groups (FIG. 56) despite the heterogeneity in the two groups (FIG. 57). Fifteen proteins were significantly decreased in the abortion group (q<0.05), including pappalysin-1 (PAPPA), pro-epidermal growth factor (EGF), interleukin-27 (IL27), placenta growth factor (PGF), follistatin (FS), growth/differentiation factor 15 (GDF15), growth hormone (GH), insulin-like growth factor-binding protein 1 (IGFBP1), carboxypeptidase A2 (CPA2), brevican core protein (BCAN), matrix metalloproteinase-12 (MMP12), channel-activating protease 1 (PRSS8), testican-1 (SPOCK1), trem-like transcript 2 protein (TLT2), trefoil factor 3 (TFF3). Five proteins were significantly elevated in the abortion group compared to controls (q<0.05). perlecan (PLC), tumor necrosis factor receptor superfamily member 11A (TNFRSF11A), interleukin-1 receptor-like 2(IL1RL2), prolargin (PRELP) and BMP-6 were significantly elevated in the abortion group versus controls (q<0.05). Importantly, brevican core protein (BCAN), carboxypeptidase A2 (CPA2), trem-like transcript 2 (TLT2) and TNFRSF11A were identified as four novel protein candidates that may play roles in mechanisms underlying human spontaneous abortion.

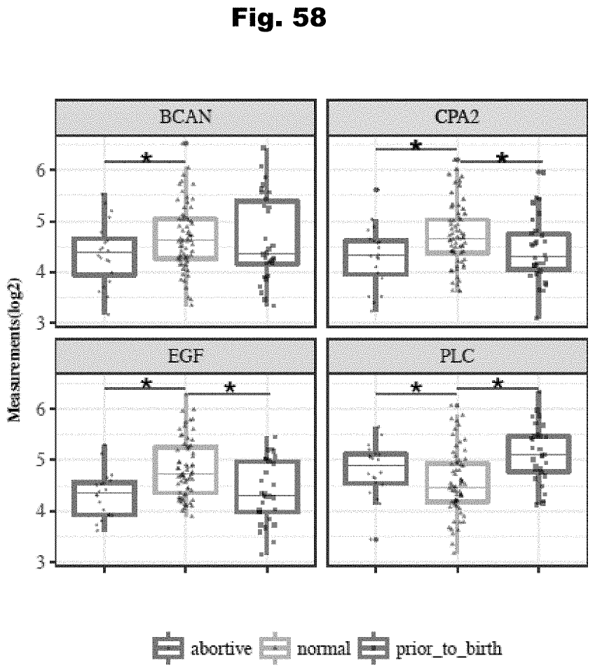

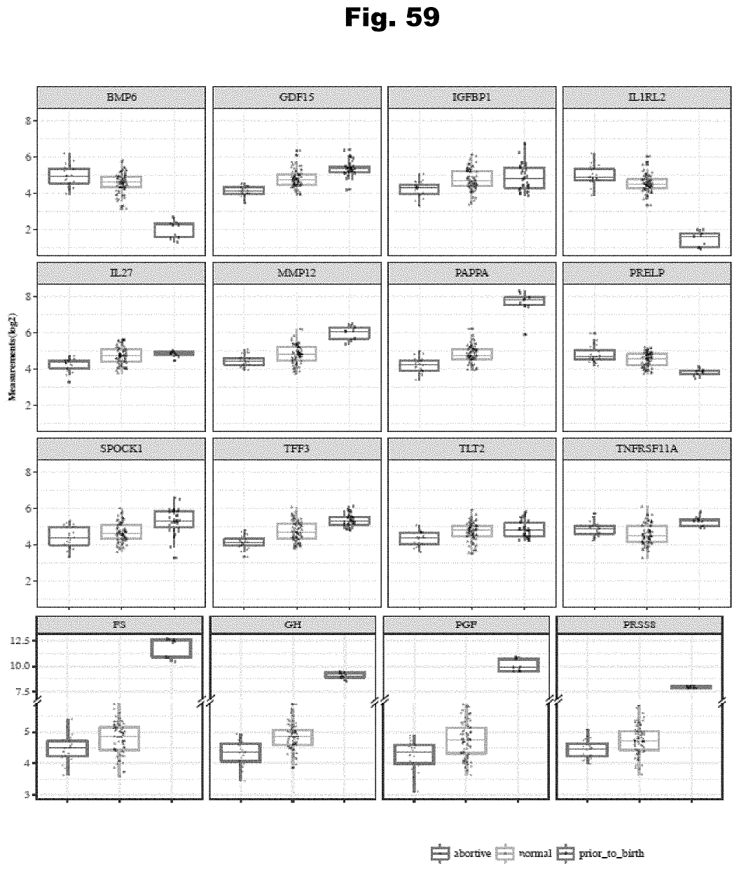

To explore whether these 20 proteins were specifically associated with spontaneous abortion or reflected conclusion of pregnancy more broadly, levels of the 20 proteins with their levels in samples collected one week before parturition in normal pregnancy were also compared. Four of the 20 proteins (BCAN, CPA2, EGF and PLC, FIG. 58) in the abortion group (abortive) were similar to those of samples collected prior to birth (prior-to-birth) but differed significantly compared to first trimester samples from normal pregnancies (normal), indicating that these proteins may play roles in the termination of pregnancy. In contrast, the other sixteen proteins showed significant changes (q<0.05) in the abortion group when compared with controls collected in the first trimester of normal pregnancies and their levels in samples prior to birth, respectively, namely BMP6, GDF15, IGFBP1, IL1RL2, IL27, MMP12, PAPPA, PRELP, SPOCK1, TFF3, TLT2, TNFRSF11A, FS, GH, PGF and PRSS8 (FIG. 59). These 16 proteins could play a role related to spontaneous abortion.

### Experimental Procedures

### Sample Preparation

Samples in this study originate from the pregnancy cohort “Biological Signals in Pregnancy” initiated by Statens Serum Institut (SSI), Denmark. In the study blood samples are collected weekly during pregnancy and once postpartum. The blood samples were collected into a K2EDTA-coated Vacutainer tube and processed within 24 hours of sample collection. Plasma was separated from blood using standard clinical blood centrifugation protocol. Sample collection and preparation were done at SSI. The Danish National Committee on Health Research Ethics has approved the study (j.no. H-3-2014-004), and written consent was collected for all participants. For this study sampling time and frequency for all participants as well as clinical information is listed in Table 4.

### Plasma Protein Profiling

Proteins were quantified in all plasma samples using multiplex proximity extension assays (Proseek Multiplex, Olink Biosciences) according to the manufacturer's instructions. For the longitudinal study four panels of a total 363 unique proteins were analyzed across pregnancy: cardiovascular disease (CVD) II, inflammation, oncology II and neurology. For the labor-associated study and that of spontaneous abortions, 436 proteins were measured with 5 panels: cardiovascular disease (CVD) II, CVD III, inflammation, oncology II and neurology. Because in addition to 6 controls in each run 90 samples were analyzed, several runs were performed to analyze all the samples in the studies. Briefly, all reactions were performed in wells of a 96-well plate, a 3 μL incubation solution, containing pairs of protein-specific antibodies conjugated with distinct barcoded oligonucleotides for each of 96 proteins and controls, was mixed with 1 μL of plasma sample and then incubated overnight at 4° C. Next, 96 μL of an extension solution containing extension enzymes and PCR reagents was added, and the plate was then incubated in a thermal cycler for extension (50° C., 20 min) and preamplification (95° C. 30 min, 17 cycles for 95° C. 30 sec, 54° C. 1 min and 60° C. 1 min). Meanwhile, a 96.96 dynamic array IFC (Fluidigm) was prepared and primed according to the manufacturer's instructions, and 2.8 μL of the extension mix was combined with 7.2 μL of detection solution in a new 96-well plate. Lastly, 5 μL of the mix was loaded to the primed 96.96 Dynamic Array IFC and 5 μL of each the 96 primer pairs were loaded to the other side of the 96.96 Dynamic Array IFC. The program for protein expression was run on a Fluidigm Biomark using the provided Proseek program (Olink Proteomics).

Ct-values (log 2 scale) of individual sample reaction were subtracted by the Ct value for the internal control for the corresponding samples, thus generating delta Ct (dCt). The dCt value was subtracted from the background reaction (a negative control), resulting in a ddCt values, and these were then used for subsequent data analyses in R and visualization with ggplot2, and in Python 3.

### Statistical Analysis

To remove batch effects, all protein data were normalized with quantile and combat normalizations. Significance calculation (q<0.05) in this study was performed with a nonparametric statistical test (Mann-Whitney U test) and Gene Ontology (GO) terms were analyzed with BiNGO (see S. Maere, K. Heymans, and M. Kuiper, Bioinformatics 21, 3448-3449 (2005), the disclosure of which is incorporated herein by reference) or by weighted gene co-expression network analysis (WGCNA) (B. Zhang and S. Horvath, Statistical applications in genetics and molecular biology 4, Article 17 (2005), the disclosure of which is incorporated herein by reference). For clustering analysis, the optimal clustering number was determined with a bootstrap approach unless otherwise noted.

WGCNA was performed for unsupervised co-expression module discovery. Considering the potential inhibitory and activating functions of proteins in this study, the scale-free overlap matrix was determined using the adjacency of unsigned network using an empirically defined soft threshold power of 6, and co-expressing modules were defined from the network. For individual identified modules of co-expressed proteins, eigengenes were computed with moduleEigengenes in WGCNA, then, correlations between the module eigengenes and clinical parameters were calculated and their corresponding p values were calculated and adjusted (Benjamini-Hochberg method) to be q values.

To analyze data on the basis of gestational month and identify groups of proteins based on their dynamic patterns across pregnancy and postpartum timepoint, average values for particular proteins of individual participants was considered in each gestational month, then analyzed using a fuzzy C-means clustering algorithm (R package “e1071”, default m value of 2) (N. R. Pal, J. C. Bezdek, and R. J. Hathaway, Neural Networks 9, 787-796 (1996), the disclosure of which is incorporated herein by reference), with clusters and patterns visualized using heatmaps. C-means membership value was assigned as the alpha value in ggplot2 and protein trends across pregnancy were visualized with an alpha value of more than 0.6.

Predictive analysis using EN algorithm was performed with scikit-learn library in Python (Jupyter notebook). First, data was divided into training and testing datasets (ratio=7:3). The training dataset was used to optimize Alpha and L1 values, and 40 essential features (proteins) were determined based on their coefficients in regression analysis. After developing the EN module with optimal alpha and L1 values, the module was validated on the testing dataset. Model predictive performance was evaluated using two matrices: Pearson correlation coefficient and root mean squared log error (RMSLE).

GO term analysis was performed in BiNGO and redundant GO terms were removed with GO trimming. To analyze labor associated proteins detected in 30 samples prior to labor and 29 postpartum samples, unsupervised hierarchical clustering, K-means and fuzzy C-means clustering were performed to determine the pattern and clusters of all protein levels before and after labor. For abortion case and controls, data was averaged for individual abortion cases and controls, and nonparametric statistical tests were performed to identify the significant proteins (q<0.05).

### Example 3: Combination of Metabolite and Protein Constituent Features

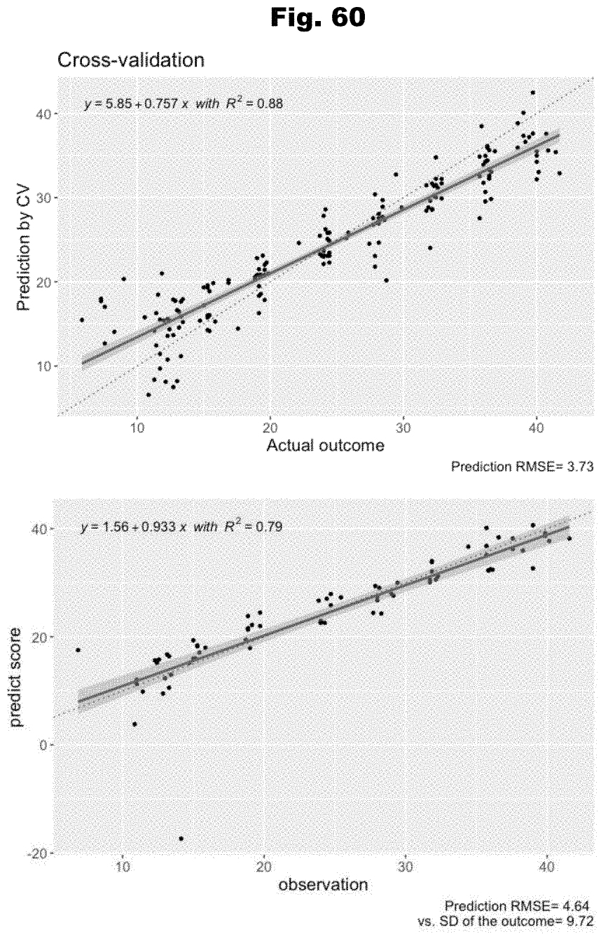

Provided in FIG. 60 are the results of model that combines metabolites and protein constituents to predict gestational age. Utilizing the Danish cohort of women, metabolite and protein samples were extracted and measured as described in Examples 1 and 2. Utilizing these measurements, a LASSO model was built combining metabolite and protein constituent features. As can be seen in FIG. 60, the combination of metabolites and protein constituents provides a robust prediction of gestational age (5 to 42 weeks).

In this model, a total of eight features were utilized, including four metabolites and four protein constituents. The four metabolites utilized were THDOC, progesterone, estriol-16-glucorinide, and DHEA-S. The four protein constituents utilized were LAIR-2, DLK-1, GRN, and PAI1. The contribution of each metabolite to the prediction power is shown in FIG. 61.

TABLE 1
Demographics and birth characteristics of the discovery and validation cohorts.
Values are means (SDs) or numbers (percentages).

| | Discovery | | Validation-1 | | Validation-2 | |
|---|---|---|---|---|---|---|
| | N = 21 | | N = 9 | | N = 8 | |
| Demographics | | | | | | |
| Maternal age at birth - years | 29.8 | (3.1) | 29.7 | (3.3) | 31.4 | (1.0) |
| Previous births - no. | | | | | | |
| 0 | 13 | (61.9) | 6 | (66.7) | 4 | (50) |
| 1 | 8 | (38.1) | 2 | (22.2) | 3 | (37.5) |
| >=2 | 0 | (0) | 1 | (11.1) | 1 | (12.5) |
| Pre-pregnancy BMI - kg/m2 | 22.1 | (2.9) | 21.2 | (3.4) | 21.1 | (1.6) |
| Smoking during pregnancy - no. | | | | | | |
| Yes | 0 | (0) | 0 | (0) | 1 | (12.5) |
| No | 18 | (85.7) | 9 | (100) | 6 | (75) |
| Missing | 3 | (14.3) | 0 | (0) | 1 | (12.5) |
| Alcohol during pregnancy - no. | | | | | | |
| Yes | 5 | (23.8) | 1 | (11.1) | 1 | (12.5) |
| Average number of units/week | 0.8 | | 1 | | 0.25 | |
| No | 13 | (61.9) | 8 | (88.9) | 6 | (75) |
| Missing | 3 | (14.3) | 0 | (0) | 1 | (12.5) |
| Birth characteristics | | | | | | |
| Gestational age - days | 281 | (8.4) | 280.7 | (8.3) | 279.3 | (9.5) |
| Mode of delivery - no. | | | | | | |
| Spontaneous vaginal birth | 10 | (47.6) | 5 | (55.6) | 4 | (50) |
| Induced vaginal birth | 7 | (33.3) | 1 | (11.1) | 3 | (37.5) |
| Sectio before onset of labour | 1 | (4.8) | 3 | (33.3) | 1 | (12.5) |
| Sectio during labour | 3 | (14.3) | 0 | (0) | 0 | (0) |
| Birth weight - gram | 3,638 | (500) | 3,803 | (662) | 3,362 | (493) |
| Birth length - centimeter | 52.4 | (2) | 53.3 | (2) | 51 | (2.3) |
| Gender of child - no. | | | | | | |
| Male | 9 | (42.9) | 5 | (55.6) | 5 | (62.5) |
| Female | 12 | (57.1) | 4 | (44.4) | 3 | (37.5) |

TABLE 2
Metabolites significantly asscoiated with pregnancy progression

| Metabolites | Pathways |
|---|---|
| Ketoisovaleric acid | Amino acid metabolism |
| Valylhistidine | Amino acid metabolism |
| Taurochenodeoxycholate | Bile acid biosythesis |
| Glycochenodeoxycholate | Bile acid biosythesis |
| 7alpha,24-Dihydroxy-4-cholesten-3-one | Bile acid biosythesis |
| Theobromine | Caffeine metabolism |
| Theophylline | Caffeine metabolism |
| 1-Methyoxanthine | Caffeine metabolism |
| Cyclo(leucylprolyl) | Caffeine metabolism |
| Caffiene | Caffeine metabolism |
| Hexadecadienoylcarnitine | Fatty acid metabolism |
| MG(20:0) | Fatty acid metabolism |
| MG(14:1) | Fatty acid metabolism |
| MG(24:1) | Fatty acid metabolism |
| MG(24:0) | Fatty acid metabolism |
| MG(18:1) | Fatty acid metabolism |
| Tetracosahexaenoic acid | Fatty acid metabolism |
| MG(22:2) | Fatty acid metabolism |
| Docosadienoic acid | Fatty acid metabolism |
| Tetracosapentaenoic acid | Fatty acid metabolism |
| Glycyrrhetinic acid | Fatty acid metabolism |
| 8,9-DHET | Fatty acid metabolism |
| beta-Glycyrrhetinic acid | Fatty acid metabolism |
| 17,18-EpETE | Fatty acid metabolism |
| Dodecanoylcarnitine | Fatty acid metabolism |
| Oleoylcarnitine | Fatty acid metabolism |
| C16 PAF (Platelet-activating factor) | Fatty acid metabolism |
| Erucic acid | Fatty acid metabolism |
| Tricosanoic acid | Fatty acid metabolism |
| Isobutyryl-L-carnitine | Fatty acid metabolism |
| 3-Hydroxyoleylcarnitine | Fatty acid metabolism |
| Tetracosatetraenoic acid | Fatty acid metabolism |
| 7-Methylguanine | Others |
| 2-Phenylbutryic acid | Others |
| Hydroxybupropion | Others |
| 3-Acetoxypyridine | Others |
| N-Acetyl-D-glucosamine | Others |
| Sinapyl alcohol | Others |
| Sphingosine | Others |
| LPC(P-18:1) | Phospholipid metabolism |
| PE(P-16:0e/0:0) | Phospholipid metabolism |
| LPC(P-16:0) | Phospholipid metabolism |
| LPC(24:0) | Phospholipid metabolism |
| LPE(22:2) | Phospholipid metabolism |
| LPC(18:2) | Phospholipid metabolism |
| LPE(22:1) | Phospholipid metabolism |
| LPE(22:4) | Phospholipid metabolism |
| LPE(20:3) | Phospholipid metabolism |
| LPE(20:0) | Phospholipid metabolism |
| LPE(20:1) | Phospholipid metabolism |
| PC(22:1/22:1) (Lecithin) | Phospholipid metabolism |
| LPC(P-18:0) | Phospholipid metabolism |
| LPC(17:0) | Phospholipid metabolism |
| PC(18:1(9Z)e/2:0) | Phospholipid metabolism |
| LPC(20:3) | Phospholipid metabolism |
| Corticosterone | Steroid hormone biosynthesis |
| Pregnenolone sulfate | Steroid hormone biosynthesis |
| Estriol-16-Glucuronide | Steroid hormone biosynthesis |
| Estrone 3-sulfate | Steroid hormone biosynthesis |
| Dehydroisoandrosterone sulfate (DHEA-S) | Steroid hormone biosynthesis |
| 3-Pregnane-3,17-diol-20-one 3-sulfate | Steroid hormone biosynthesis |
| Androsterone sulfate | Steroid hormone biosynthesis |
| 17alpha-Hydroxyprogesterone | Steroid hormone biosynthesis |
| THDOC | Steroid hormone biosynthesis |
| Androstane-3-17-diol | Steroid hormone biosynthesis |
| Progesterone | Steroid hormone biosynthesis |
| Cortisone | Steroid hormone biosynthesis |
| Cortisol | Steroid hormone biosynthesis |

TABLE 3
Metabolite Features selected by Gestational Age Machine Learning Model

| Metabolic feature | Contribution | m/z | RT/min | Polarity | Compound name | MSI confidence level |
|---|---|---|---|---|---|---|
| M1 | 0.15640256 | 399.148186 | 6.70403333 | negative | N,N′-Dicarbobenzyloxy- | 3 |
| | | | | | L-ornithine | |
| M2 | 0.10626678 | 438.297358 | 9.52088333 | positive | PE(P-16:0e/0:0) | 1 |
| M3 | 0.07564218 | 413.3057 | 10.9363333 | negative | delta4-Dafachronic acid | 2 |
| M4 | 0.06705228 | 529.241193 | 8.02455 | positive | C29H36O9 | 4 |
| M5 | 0.05015041 | 510.928964 | 5.38005 | positive | | |
| M6 | 0.0461547 | 417.335441 | 10.47695 | positive | 7alpha,24-Dihydroxy-4- | 2 |
| | | | | | cholesten-3-one | |
| M7 | 0.04528347 | 531.256239 | 7.73061667 | positive | C22H43O12P | 4 |
| M8 | 0.04310647 | 511.290422 | 7.818 | negative | C27H44O9 | 4 |
| M9 | 0.03430518 | 399.148108 | 5.71296667 | negative | C13H28O7S | 4 |
| M10 | 0.03073771 | 257.226065 | 8.3289 | positive | Androstane-3,17-diol | 3 |
| M11 | 0.02803161 | 315.23144 | 7.57591667 | positive | 21-Hydroxypregnenolone | 3 |
| M12 | 0.02468241 | 519.25644 | 8.63863333 | positive | | |
| M13 | 0.02443109 | 563.179693 | 6.59151667 | positive | | |
| M14 | 0.02436834 | 463.196759 | 6.66445 | negative | Estriol-16-Glucuronide | 1 |
| M15 | 0.02206127 | 353.208453 | 7.91116667 | positive | | |
| M16 | 0.01910817 | 487.193529 | 6.60698333 | positive | | |
| M17 | 0.01819669 | 483.259261 | 8.3339 | negative | C25H40O9 | 4 |
| M18 | 0.01672713 | 431.315278 | 9.77563333 | negative | C27H44O4 | 4 |
| M19 | 0.0141188 | 415.319161 | 9.4745 | positive | C27H42O3 | 4 |
| M20 | 0.0135292 | 301.252207 | 7.73576667 | positive | bilobol | 3 |
| M21 | 0.0133291 | 331.226501 | 7.92145 | positive | [1-(3,5-dihydroxyphenyl)- | 2 |
| | | | | | 12-hydroxytridecan-2-yl) ace | |
| M22 | 0.01275839 | 538.350052 | 8.87598333 | positive | C26H52NO8P | 4 |
| M23 | 0.01264703 | 493.279871 | 9.13475 | negative | C27H42O8 | 4 |
| M24 | 0.01202183 | 263.138753 | 1.891 | positive | Propylphenylalanine | 2 |
| M25 | 0.01187665 | 371.188422 | 9.70106667 | negative | N,N,Diacetyl-Lys-DAIs-DAIs | 2 |
| M26 | 0.00971169 | 465.344852 | 7.39026667 | positive | C23H49N2O5P | 4 |
| M27 | 0.00927896 | 297.220982 | 7.42121667 | positive | C21H29O | 4 |
| M28 | 0.00920426 | 593.369171 | 10.17005 | negative | C33H53O9 | 4 |
| M29 | 0.0090048 | 347.25906 | 9.52455 | negative | C22H35O3 | 4 |
| M30 | 0.00767647 | 498.303676 | 9.25845 | negative | C30H44NO3S | 4 |
| M31 | 0.0061576 | 319.164786 | 2.57961667 | positive | | |
| M32 | 0.00600745 | 401.16376 | 7.8948 | negative | Glycine, 1,1′-(1,8- | 3 |
| | | | | | dioxo-1,8- | |
| | | | | | octanediyl)bis[glycyl- | |
| M33 | 0.00583855 | 525.269422 | 6.33488333 | negative | C27H2O10 | 4 |
| M34 | 0.0050616 | 381.101128 | 5.38683333 | negative | 6-ketoestriol sulfate | 2 |
| M35 | 0.00356451 | 315.231485 | 9.29935 | positive | Progesterone | 1 |
| M36 | 0.00175375 | 821.296071 | 9.12933333 | negative | | |
| M37 | 0.00125418 | 527.285199 | 8.26256667 | negative | DAH-3-Keto-4-en | 3 |
| M38 | 0.00088668 | 653.286204 | 9.25845 | negative | | |
| M39 | 0.00081733 | 798.35665 | 8.45273333 | positive | | |
| M40 | 0.00032228 | 260.106419 | 9.77563333 | negative | | |
| M41 | 0.00027414 | 823.311537 | 9.25845 | negative | | |
| M42 | 0.00019631 | 337.21348 | 9.29935 | positive | Progesterone | 3 |

Participants, clinical data and
their analyses in protein panels

| Analysis of 4 | | | | | | | | | | | | | | | |
|---|---|---|---|---|---|---|---|---|---|---|---|---|---|---|---|
| panel | | | | | | | | | | | | | | | |
| proteins* | | | | | | | | | | | | | | | |
| Analysis of 5 | | | | | | | | | | | | | | | |
| panel | | | | Yes | | | | | | | | Yes | | | |
| proteins* | | | | Gestation week | | | | | | | | Gestation week | | | |
| Individual | | | | (Trimester 1) | | | | | | | | (Trimester 2) | | | |
| #1 | | | | | | | | | | | | | 15.1 | 19.1 | 24 |
| #2 | | | | | | | | | | | 12.3 | 13.9 | 15.6 | 19.4 | 24.4 |
| #3 | 6.9 | | | | 9.1 | | | | 11 | | 12.3 | 13.1 | 15 | 18.9 | 24 |
| #5 | 7.1 | | | | | | | | 11 | | | 13 | 15 | 18.9 | 23.9 |
| #7 | | | | | | | | | | | | 13.3 | 15.3 | 19 | 24 |
| #9 | 7.7 | | | | | | | | 11.4 | | 12.4 | 13.4 | 15.4 | 18.9 | 24.4 |
| #10 | | | | | | | | | | | | | 15.3 | 19.4 | 24.6 |
| #11 | 7.2 | | | | | | | | 11.4 | | 12.1 | 13.1 | 15.1 | 19.5 | 24.1 |
| #12 | | | | | | | | | | | | 13.3 | 15.1 | 18.7 | 24.7 |
| #13 | | | | | | | | 10.9 | | | 12.9 | | 14.7 | 19 | 24.3 |
| #15 | | | | | | | | | 11.4 | | 12.2 | 13.2 | 15.2 | 19.1 | 24 |
| #16 | 7.2 | | | | | | | | 11.5 | | 12 | 13 | 15.2 | 19.4 | 24 |
| #17 | | | | | | | | 10.8 | | v | 12.7 | | 15 | 19.1 | 24 |
| #18 | 8.2 | | | | 9.1 | 9.8 | 10 | 10.4 | | | 12 | 13.1 | 15.2 | 19.5 | 24 |
| #19 | | | | | | | | | | | | | | 22.1 | 24.1 |
| #20 | | | | | | | | | | | | | | | 25.8 |
| #21 | 7.2 | | | | | | | | 11.2 | | 12.2 | 13.2 | 15.2 | 19.2 | 24.4 |
| #22 | 5.8 | | | | | | | | | 11.7 | 12.4 | | 15.7 | 19.2 | 25.7 |
| #23 | | | | | | | | | | | | 13.2 | 15.4 | 19.5 | 24.4 |
| #24 | 9 | | | | | | | | 11.2 | 11.7 | 12.7 | | 16.8 | 19.1 | 24.2 |
| #25 | | | | | | | | | | | | 13 | 15.8 | 17.5 | 23.7 |
| #26 | | | | | | | | | | | | | 15.4 | 18.8 | 24.4 |
| #28 | 7.5 | | | | | | | | 11.4 | | 12.7 | 13.4 | 14.7 | 19.5 | 24.2 |
| #29 | | | | | | | | | | | | | 16.8 | 19.5 | 24.5 |
| #30 | | | | | | | | | | | | | | 19.7 | 23.7 |
| #32 | | | | | | | 10.5 | | | 11.8 | 12.8 | | 15 | 18.8 | 23.8 |
| #33 | | | | | | | | | | | | | | 19.1 | 24.4 |
| #34 | | | | | | | | | | | | 13.1 | 15.2 | 19.1 | 24.2 |
| #42 | | | | | | | | | | | | | | 19 | 24 |
| #44 | | | | | | | | | | | | | | 19 | 24.1 |
| #36 | 7.8 | 8 | 8.5 | 8.8 | 9 | 9.8 | | | 11 | 11.8 | 12.4 | N/I | N/I | N/I | N/I |
| #41 | 7.5 | | 8.5 | 8.8 | | 9.5 | | | | | | N/I | N/I | N/I | N/I |
| #58 | 7.5 | | 8.5 | 8.3 | | 9.8 | | | | | | N/I | N/I | N/I | N/I |
| #60 | | | | 8.7 | | 10 | 10.4 | 10.7 | 11.4 | | 12.4 | N/I | N/I | N/I | N/I |
| #63 | | | | 8.8 | 9.1 | 10 | | | 11.4 | | | N/I | N/I | N/I | N/I |
| #102 | | | | | | 9.8 | | | | 11.7 | | N/I | N/I | N/I | N/I |
| #6 | | | | | | | | 10.9 | | 11.7 | 12.9 | | | | |
| #14 | | | | 8.8 | | 9.8 | | | | | | | | | |
| #27 | | 8 | | | 9 | | | | | | | | | | |
| #43 | 7.7 | | | 8.7 | | | | | | | | | | | |
| #62 | | | | | 9 | 9.8 | | | | 11.8 | | | | | |
| #107 | 6.8 | | 8.5 | | | 9.5 | | 10.7 | | 11.8 | | | | | |
| #114 | 7.5 | | | | | | | | | | | | | | |

Participants, clinical data and
their analyses in protein panels

| Analysis of 4 | | | | | | | |
|---|---|---|---|---|---|---|---|
| panel | | | | | | | |
| proteins* | | | | Yes | | | |
| Analysis of 5 | | | | Gestation week | | | |
| panel | Yes | | | (Trimester 3) | | | Birth/ |
| proteins* | Gestation week | | | (prior-to birth | | Yes | Spontaneous |
| Individual | (Trimester 3) | | | sample) | | Postpartum | Abortion(SA) |
| #1 | 27.9 | 32.1 | 36.1 | | 39 | 42.1 | Singleton |
| #2 | 27.7 | 31.6 | 35.4 | 37.3 | | 42.3 | Singleton |
| #3 | 28.1 | 31.9 | 35.9 | | 39.9 | 43.9 | Singleton |
| #5 | 28 | 31.9 | 35.9 | | 40.1 | 42.7 | Singleton |
| #7 | 29 | 32 | 36 | | 39.9 | 42.1 | Singleton |
| #9 | 28.3 | 32.3 | 36.6 | 38.3 | | 41.4 | Singleton |
| #10 | 28.4 | 31.3 | 36.4 | | 40.4 | 41.6 | Singleton |
| #11 | 28.1 | 32.1 | 36.1 | 38.2 | | 41.4 | Singleton |
| #12 | 29.1 | 31.7 | 35.7 | 39 | | 43.1 | Singleton |
| #13 | 27.7 | 31.7 | 35.7 | 38.7 | | 39.9 | Singleton |
| #15 | 27.8 | 31.8 | 36.1 | | 40 | 43 | Singleton |
| #16 | 28.1 | 32.2 | 36.2 | 39 | | 41.2 | Singleton |
| #17 | 27.8 | 31.8 | 35.7 | | 41.7 | 46.1 | Singleton |
| #18 | 29 | 32.1 | 36 | | 40 | 43.2 | Singleton |
| #19 | 28.1 | 32 | 36.1 | 39.1 | 40.1 | | Singleton |
| #20 | 27.7 | 31.7 | 36.5 | | 41.4 | 45.5 | Singleton |
| #21 | 27.4 | 32.2 | 36.5 | | 40.4 | 43.4 | Singleton |
| #22 | 28.7 | 32.4 | 36.2 | | | 40.7 | Singleton |
| #23 | 28.4 | 32.8 | 36.2 | 39.7 | | 43.2 | Singleton |
| #24 | 29.8 | 32.1 | 35.7 | | 40.7 | 46 | Singleton |
| #25 | 28.1 | 32 | 36.1 | | 40 | 42 | Singleton |
| #26 | 27.8 | 32.1 | 36.2 | 36.8 | | 41.1 | Singleton |
| #28 | 28.4 | 32.4 | 36.4 | 38.4 | 40.8 | 39.4 | Singleton |
| #29 | 27.7 | 32.8 | 35.7 | | | 42.7 | Singleton |
| #30 | 28 | 31.7 | 35.8 | 39 | | 42.7 | Singleton |
| #32 | 23.5 | 32.5 | 36 | 39.7 | | 42.5 | Singleton |
| #33 | 28.4 | 32.2 | 36.4 | | 40 | 44.2 | Singleton |
| #34 | 28.1 | 32.2 | 36.1 | 39.4 | | 42.2 | Singleton |
| #42 | | 32.8 | | 38.5 | | 42.8 | Singleton |
| #44 | 29.4 | 32.4 | 36.4 | 37.4 | | 41.4 | Singleton |
| #36 | N/I | N/I | N/I | N/I | N/I | N/I | Singleton |
| #41 | N/I | N/I | N/I | N/I | N/I | N/I | Singleton |
| #58 | N/I | N/I | N/I | N/I | N/I | N/I | Singleton |
| #60 | N/I | N/I | N/I | N/I | N/I | N/I | Singleton |
| #63 | N/I | N/I | N/I | N/I | N/I | N/I | Singleton |
| #102 | N/I | N/I | N/I | N/I | N/I | N/I | Singleton |
| #6 | | | | | | | SA |
| #14 | | | | | | | SA |
| #27 | | | | | | | SA |
| #43 | | | | | | | SA |
| #62 | | | | | | | SA |
| #107 | | | | | | | SA |
| #114 | | | | | | | SA |

*N/I: sample not included
*4-panel proteins: cardiovascular disease (CVD) II, inflammation, oncology II and neurology
*5-panel proteins: cardiovascular disease (CVD) II, CV III, inflammation, oncology II and neurology

TABLE 4
Proteins in individual fuzzy c-means clusters

| | | |
|---|---|---|
| Protein | UniProt | Name |

Cluster 1

| | | |
|---|---|---|
| CEACAM1 | P13688 | Carcinoembrynic antigen-related cell adhesion molecule 1 |
| MSLN | Q13421 | Mesothelin |
| TNFRSF6B | Q95407 | TNF receptor superfamily member 6B |
| TGFR2 | P37173 | TGF-beta receptor type 2 |
| CD48 | P09326 | CD 48 antigen |
| hK11 | Q9UBX7 | Kallikrein-11 |
| GPC1 | P35052 | Glypican-1 |
| TFPI2 | P48307 | Tissue factor pathway inhibitor 2 |
| hK8 | O60259 | Kallikrein-8 |
| VEGFR2 | P15692 | Vascular endothelial growth factor A |
| PODXL | O00592 | Podocalyxin |
| IGF1R | P08069 | Insulin-like growth factor 1 receptor |
| SPARC | P09486 | Osteonectin |
| GZMH | P20718 | Granzyme H |
| TGFalpha | P01135 | Transforming growth factor alpha |
| FASLG | P48023 | Fas antigen ligand |
| EPHA2 | P29317 | Ephrin type-A receptor 2 |
| SEZ6L | Q9BYH1 | Seizure 6-like protein |
| CAP | Q16790 | Carbonic anhydrase IX |
| MIA | Q16674 | Melanoma-derived growth regulatory proteinÊ |
| CTSV | O60911 | Cathepsin L2 |
| CD27 | P26842 | CD27 antigen |
| XPNPEP2 | O43895 | Xaa-Pro aminopeptidase 2 |
| ERBB4 | Q15303 | Receptor tyrosine-protein kinase ErbB-4Ê |
| ADAM8 | P78325 | Disintegrin and metalloproteinase domain-containing protein 8 |
| DLL1 | O00548 | Delta-like protein 1 |
| FGFBP1 | Q14512 | Fibroblast growth factor-binding protein 1 |
| VIM | P08670 | Vimentin |
| CD160 | O95971 | CD160 antigen |
| MIC-A/B | Q29983, Q29980 | MHC class I polypeptide-related sequence A/BÊ |
| S100A11 | P31949 | Protein S100-A11 |
| AR | P10275 | Androgen receptor (Dihydrotestosterone receptor) |
| CD207 | Q9UJ71 | C-type lectin domain family 4 member K |
| ICOSLG | O75144 | ÊICOS ligand |
| WFDC2 | Q14508 | WAP four-disulfide core domain protein 2 |
| CXCL13 | O43927 | C-X-C motif chemokine 13 |
| CD70 | P32970 | CD70 antigen |
| FRgamma | P41439 | Folate receptor gammaÊ |
| CEACAM5 | P06731 | Carcinoembryonic antigen (CEA) |
| ANXA1 | P04083 | Annexin A1Ê |
| ITG82 | P05107 | Integrin beta-2 |
| TNFR2 | P20333 | Tumor necrosis factor receptor 2Ê |
| MMP9 | P14780 | Matrix metalloproteinase-9Ê |
| IL2RA | P01589 | Interleukin 2 receptor subunit alpha |
| ALCAM | Q13740 | CD166 antigen |
| Gal3 | P17931 | Galectin-3 |
| PLC | P98160 | Perlecan |
| CDH5 | ÊP33151 | Cadherin-5 |
| TNFRSF10C | ÊO14798 | Tumor necrosis factor receptor superfamily member 10CÊ |
| SELE | P16581 | E-selectin |
| AZU1 | P20160 | Azurocidin |
| IL6RA | P08887 | Interleukin-6 receptor subunit alpha |
| RETN | Q9HD89 | Resistin |
| IGFBP1 | P08833 | Insulin-like growth factor-binding protein 1Ê |
| CHIT1 | Q13231 | Chitotriosidase 1 |
| AXL | ÊP30530 | Tyrosine-protein kinase receptor UFOÊ |
| PRTN3 | P24158 | Myeloblastin |
| PGLYRP1 | O75594 | Peptidoglycan recognition protein 1 |
| CPA1 | ÊP15085 | Carboxypeptidase A1 |
| IL1RT2 | P14778 | Interleukin-1 receptor type 1 |
| SHPS1 | P78324 | Tyrosine-protein phosphatase non-receptor type substrate 1 |
| CPB1 | P15086 | Carboxypeptidase B |
| SCGB3A2 | Q96PL1 | Secretoglobin family 3A member 2 |
| MMP3 | P08254 | Matrix metalloproteinase-3 |
| RARRES2 | Q99969 | Retinoic acid receptor responder protein 2 |
| NTproBNP | | ÊN-terminal of the prohormone brain natriuretic peptide(NT-proBNP) |
| VEGFA | P15692 | Vascular endothelial growth factor A |
| MCP3 | P80098 | Monocyte chemotactic protein 3 |
| CDCP1 | Q9H5V8 | CUB domain-containing protein 1Ê |
| IL6 | P05231 | Interleukin-6Ê |
| IL17C | Q9P0M4 | Interleukin-17C (IL-17C) |
| IL17A | Q16552 | Interleukin-17A (IL-17A) |
| CXCL11 | O14625 | C-X-C motif chemokine 11 (CXCL11) |
| IL20RA | Q9UHF4 | Interleukin-20 receptor subunit alpha (IL-20RA) |
| CXCL9 | Q07325 | C-X-C motif chemokine 9 (CXCL9) |
| CST5 | P28325 | Cystatin D |
| IL2RB | P01589 | Interleukin-2 receptor subunit alpha (IL2-RA) |
| IL1alpha | P01583 | Interleukin-1 alpha (IL-1 alpha) |
| OSM | P13725 | Oncostatin-MÊ |
| IL2 | P60568 | Interleukin-2Ê |
| TSLP | Q969D9 | Thymic stromal lymphopoietin |
| CCL4 | P13236 | C-C motif chemokine 4 |
| CD6 | Q8WWJ7 | T cell surface glycoprotein CD6 isoform |
| IL18 | Q14116 | Interleukin-18 |
| SLAMF1 | Q13291 | Signaling lymphocytic activation moleculeÊ |
| TGFA | P01135 | Transforming growth factor alpha (TGF-alpha) |
| IL10RA | Q13651 | Interleukin-10 receptor subunit alpha (IL-10RA) |
| FGF5 | Q8NF90 | Fibroblast growth factor 5 (FGF-5) |
| MMP1 | P03956 | Matrix metalloproteinase-1 (MMP-1) |
| FGF21 | Q9NSA1 | Fibroblast growth factor 21 |
| IL15RA | Q13261 | Interleukin-15 receptor subunit alpha (IL-15RA) |
| IL18R1 | Q13478 | Interleukin-18 receptor 1 (IL-18R1) |
| IL24 | Q13007 | Interleukin-24 (IL-24) |
| IL13 | P35225 | Interleukin-13 (IL-13) |
| IL10 | P22301 | Interleukin-10 |
| TNF | ÊP01375 | Tumor necrosis factorÊ |
| CCL3 | P10147 | C-C motif chemokine 3 |
| Flt3L | P49771 | Fms-related tyrosine kinase 3 ligand |
| CXCL10 | P02778 | C-X-C motif chemokine 10 |
| IL20 | Q9NYY1 | Interleukin-20 (IL-20) |
| DNER | Q8NFT8 | Delta and Notch-like epidermal growth factor-related receptorÊ |
| ENRAGE | P80511 | Protein S100-A12 (EN-RAGE) |
| IL33 | O95760 | Interleukin-33 |
| IFNgamma | P01579 | Interferon gamma (IFN-gamma) |
| FGF19 | O95750 | Fibroblast growth factor 19 (FGF-19) |
| IL4 | P05112 | Interleukin-4 (IL-4) |
| LIF | P15018 | Leukemia inhibitory factor |
| NRTN | Q99748 | Neurturin |
| CCL25 | O15444 | C-C motif chemokine 25 |
| CX3CL1 | P78423 | Fractalkine |
| CCL20 | P78556 | C-C motif chemokine 20 |
| IL5 | P05113 | Interleukin-5Ê |
| TNFB | P01374 | TNF-betaÊ |
| CSF1 | P09603 | Macrophage colony-stimulating factor 1 (CSF-1) |
| NMNAT1 | Q9HAN9 | Nicotinamide/nicotinic acid mononucleotide adenylyltransferase 1 |
| BDNF | Q16620 | BDNF/NT-3 growth factors receptor (NTRK2) |
| CLM6 | Q08708 | CMRF35-like molecule 6 (CLM-6) |
| EZR | P15311 | Ezrin |
| NBL1 | P41271 | Neuroblastoma suppressor of tumorigenicity 1 |
| NCAN | O14594 | Neurocan core protein |
| CPA2 | P48052 | Carboxypeptidase A2 |
| Alpha2MRAP | P30533 | Alpha-2-macroglobulin receptor-associated protein (Alpha-2-MRAP) |
| RGM8 | Q6NW40 | RGM domain family member BÊ |
| ADAM22 | Q9P0K1 | Disintegrin and metalloproteinase domain-containing protein 22 |
| MATN3 | O15232 | Matrilin-3 |
| BCAN | Q96GW7 | Brevican core protein |
| NEP | P08473 | Neprilysin |
| THY1 | P04216 | Thy-1 membrane glycoproteinÊ |
| CDH3 | P22223 | Cadherin-3 |
| BetaNGF | P01138 | Beta-nerve growth factor (Beta-NGF) |
| CLEC10A | Q8IUN9 | C-type lectin domain family 10 member A |
| IL5Ralpha | Q01344 | Interleukin-5 receptor subunit alpha (IL-5R-alpha) |
| CDH6 | P55285 | Cadherin-6 |
| JAMB | P57087 | Junctional adhesion molecule B (JAM-B) |
| Dkk4 | Q9UBT3 | Dickkopf-related protein 4 (Dkk-4) |
| TNR | Q92752 | Tenascin-R (TN-R) |
| KYNU | Q16719 | Kynureninase |

Cluster 2

| | | |
|---|---|---|
| TXLNA | P40222 | Alpha-taxilin |
| CPE | P16870 | Carboxypeptidase E |
| KLK13 | Q9UKR3 | Kallikrein-13Ê |
| TNFSF13 | O75888 | Tumor necrosis factor ligand superfamily member 13Ê |
| EGF | P01133 | epidermal growth factor |
| SCAMP3 | O14828 | Secretory carrier-associated membrane protein 3Ê |
| LY9 | Q9HBG7 | T-lymphocyte surface antigen Ly-9 |
| ITGAV | P06756 | Integrin alpha-V |
| TNFSF10 | P50591 | Tumor necrosis factor ligand superfamily member 10 |
| S100A4 | P26447 | Protein S100-A4Ê |
| GZMH | P20718 | Granzyme H |
| hK14 | Q9P0G3 | Kallikrein-14 |
| FADD | Q13158 | FAS-associated death domain protein |
| MetAP2 | P50579 | Methionine aminopeptidase 2 |
| ITGB5 | P18084 | Integrin beta-5 |
| Gal1 | P09382 | Galectin-1 |
| DKN1A | P38936 | Cyclin-dependent kinase inhibitor 1 (CDKN1A) |
| MK | P21741 | MidkineÊ |
| ABL1 | P00519 | Tyrosine-protein kinase ABL1 |
| LYN | P07948 | Tyrosine-protein kinase Lyn |
| TNFRSF19 | Q9NS68 | Tumor necrosis factor receptor superfamily member 19 |
| TCL1A | P56279 | T-cell leukemia/lymphoma protein 1A |
| TNFRSF4 | P43489 | Tumor necrosis factor receptor superfamily member 4Ê |
| CXL17 | Q6UXB2 | VEGF-co regulated chemokine 1 (CXCL17) |
| PPY | P01298 | Pancreatic prohormone |
| ESM1 | Q9NQ30 | Endothelial cell-specific molecule 1 |
| MADhomolog5 | Q99717 | Mothers against decapentaplegic homolog 5 (MAD homolog 5) |
| ADAMTS15 | Q8TE58 | A disintegrin and metalloproteinase with thrombospondin motifs 15 |
| | | (ADAM-TS 15) |
| RSPO3 | Q9BXY4 | R-spondin-3 |
| MUC16 | Q8WXI7 | Mucin-16Ê |
| WIF1 | Q9Y5W5 | Wnt inhibitory factor 1 |
| GZMB | P10144 | Granzyme BÊ |
| CD5 | P06127 | T-cell surface glycoprotein CD5 |
| CNTN1 | Q12860 | Contactin-1Ê |
| FABP4 | P15090 | Fatty acid-binding protein, adipocyte |
| CCL24 | O00175 | C-C motif chemokine 24 |
| SPON1 | Q9HCB6 | Spondin-1 |
| MPO | P05164 | Myeloperoxidase |
| TRAP | P13686 | Tartrate-resistant acid phosphatase type 5 (TR-AP) |
| CCL22 | O00626 | C-C motif chemokine 22 |
| PSPD | P35247 | Pulmonary surfactant-associated protein D |
| PI3 | P19957 | Elafin |
| EpCAM | P16422 | Epithelial cell adhesion molecule (Ep-CAM) |
| APN | P15144 | Aminopeptidase N |
| MMP2 | P08253 | Matrix metalloproteinase-2 |
| MB | P02144 | Myoglobin |
| TNFSF13B | Q9Y275 | Tumor necrosis factor ligand superfamily member 13B |
| UPAR | Q03405 | Urokinase plasminogen activator surface receptor |
| JAMA | Q9Y624 | Junctional adhesion molecule A (JAM-A) |
| CASP3 | P42574 | Caspase-3 (CASP-3) |
| CHI3L1 | P36222 | Chitinase-3-like protein 1 |
| IGFBP7 | Q16270 | Insulin-like growth factor-binding protein 7 (IBP-7) |
| CD93 | Q9NPY3 | Complement component C1q receptor |
| COL1A1 | P02452 | Collagen alpha-1(I) chain (Alpha-1 type I collagen) |
| PON3 | Q15166 | Serum paraoxonase/lactonase 3 |
| KLK6 | Q92876 | Kallikrein-6 |
| PDGFsubunitA | P04085 | Platelet-derived growth factor subunit A (PDGF subunit A) (PDGF-1) |
| IGFBP2 | P18065 | Insulin-like growth factor-binding protein 2 (IBP-2) |
| PECAM1 | P16284 | Platelet endothelial cell adhesion molecule (PECAM-1) |
| IL8 | P10145 | Interleukin-8 (IL-8) |
| hGDNF | P39905 | Glial cell line-derived neurotrophic factor |
| CD244 | Q9BZW8 | Natural killer cell receptor 2B4 |
| IL7 | P13232 | Interleukin-7Ê |
| MCP1 | P13500 | Monocyte chemotactic protein 1(MCP-1) |
| AXIN1 | O15169 | Axin-1 |
| CXCL6 | P80162 | C-X-C motif chemokine 6 |
| SIRT2 | Q8IXJ6 | SIR2-like protein 2Ê |
| CD40 | ÊP25942 | CD40L receptorÊ |
| MCP2 | P80075 | Monocyte chemotactic protein 2 (MCP-2) |
| CASP8 | P42574 | Caspase-3 (CASP-3) |
| TNFRSF9 | Q07011 | Tumor necrosis factor receptor superfamily member 9Ê |
| NT3 | P20783 | Neurotrophin-3 (NT-3) |
| TWEAK | O43508 | Tumor necrosis factor (Ligand) superfamily, member 12 |
| ST1A1 | P50225 | Sulfotransferase 1A1 |
| STAMP5 | O95630 | STAM-binding protein |
| ADA | P00813 | Adenosine DeaminaseÊ |
| CADM3 | Q8N126 | Cell adhesion molecule 3Ê |
| GDNF | P39905 | Glial cell line-derived neurotrophic factorÊ |
| VWC2 | Q2TAL6 | BrorinÊ |
| Siglec9 | Q9Y336 | Sialic acid-binding Ig-like lectin 9 (Siglec-9) |
| ROBO2 | Q9HCK4 | Roundabout homolog 2 |
| CRTAM | O95727 | Cytotoxic and regulatory T-cell molecule |
| RGMA | Q96B86 | Repulsive guidance molecule A |
| PLXNB3 | Q9ULL4 | Plexin-B3Ê |
| CD38 | P28907 | ADP-ribosyl cyclase/cyclic ADP-ribose hydrolase 1 |
| CNTN5 | O94779 | Contactin-5 |
| CLEC1B | Q9P126 | C-type lectin domain family 1 member B |
| RSPO1 | Q2MKA7 | R-spondin-1 |
| HAGH | Q16775 | Hydroxyacylglutathione hydrolase |
| LXN | Q9BS40 | Latexin |
| gal8 | O00214 | Galectin-8 (Gal-8) |
| GDF8 | O14793 | Growth/differentiation factor 8 (GDF-8) |
| TMPRSS5 | Q9H3S3 | Transmembrane protease serine 5 |
| GFRalpha1 | P56159 | GDNF family receptor alpha-1 (GDNF receptor alpha-1) |
| SCARA5 | | |
| NTRK2 | Q16620 | BDNF/NT-3 growth factors receptor |
| GZMA | P12544 | Granzyme A |
| DRAXIN | Q8NBI3 | Draxin |
| 4EBP1 | Q13541 | Eukaryotic translation initiation factor 4E-binding protein 1 (4E-BP1) |
| SCARF2 | Q96GP6 | Scavenger receptor class F member 2 |
| GDNFRalpha3 | O60609 | GDNF family receptor alpha-3 (GDNF receptor alpha-3) |
| FLRT2 | O43155 | Leucine-rich repeat transmembrane protein FLRT2 |
| GCP5 | P78333 | Glypican-5 |
| BMP4 | P12644 | Bone morphogenetic protein 4 (BMP-4) |
| MDGA1 | Q8NFP4 | MAM domain-containing glycosylphosphatidylinositol |
| | | anchor protein 1 |
| IL12B.IL12A | P29459 | Interleukin-12 subunit alpha (IL-12A) |
| LAT | O43561 | Linker for activation of T-cells family member 1 |
| NTRK3 | Q16288 | NT-3 growth factor receptor |
| LAIR2 | Q6ISS4 | Leukocyte-associated immunoglobulin-like receptor 2 (LAIR-2) |
| MANF | P55145 | Mesencephalic astrocyte-derived neurotrophic factor |
| CD200R1 | Q8TD46 | Cell surface glycoprotein CD200 receptor 1 |
| TRAIL | P50591 | TNF-related apoptosis-inducing ligand |
| CXCL1 | P09341 | Growth-regulated alpha protein (C-X-C motif chemokine 1) |
| SCF | P21583 | Stem cell factor |
| MCP4 | Q99616 | Monocyte chemotactic protein 4 (MCP-4) |
| CCL11 | P51671 | Eotaxin |
| TNFSF14 | O43557 | Tumor necrosis factor ligand superfamily member 14Ê |
| CCL19 | Q99731 | C-C motif chemokine 19 |
| CXCL5 | P42830 | C-X-C motif chemokine 5 |
| TRANCE | O14788 | TNF-related activation-induced cytokine |
| IL12B | P29460 | Interleukin-12 subunit beta (IL-12B) |
| MMP10 | P09238 | Stromelysin-2 (SL-2) |
| CCL23 | P55773 | C-C motif chemokine 23 |
| FCRLB | Q6BAA4 | Fc receptor-like B |
| TNFRSF14 | Q92956 | Tumor necrosis factor receptor superfamily member 14 |
| IL17RA | Q96F46 | Interleukin-17 receptor A (IL-17RA) |
| SELP | P16109 | P-selectin |
| CSTB | P04080 | Cystatin-B (CPI-B) |
| MEPE | Q9NQ76 | Matrix extracellular phosphoglycoprotein |
| BLMhydrolase | Q13867 | Bleomycin hydrolase |
| Notch3 | Q9UM47 | Neurogenic locus notch homolog protein 3 |
| TIMP4 | Q99727 | Metalloproteinase inhibitor 4 |

Cluster 3

| | | |
|---|---|---|
| SYND1 | P18827 | Syndecan-1 |
| IFNgammaR1 | P15260 | Interferon gamma receptor 1 (IFN-gamma receptor 1) |
| LYPD3 | O95274 | Ly6/PLAUR domain-containing protein 3 |
| ERBB2 | P04626 | Receptor tyrosine-protein kinase erbB-2 |
| ERBB3 | P21860 | Receptor tyrosine-protein kinase erbB-3 |
| FURIN | P09958 | Furin |
| CYR61 | O00862 | Protein CYR61 |
| PVRL4 | Q96NY8 | Nectin-4 |
| GPNMB | Q14956 | Transmembrane glycoprotein NMB |
| 5′NT | P21589 | 5′-nucleotidase (5′-NT) |
| TLR3 | O15455 | Toll-like receptor 3 |
| RET | PG7949 | Proto-oncogene tyrosine-protein kinase receptor Ret |
| CRNN | Q9UBG3 | CornulinÊ |
| WISP1 | O95388 | WNT1-inducible-signaling pathway protein 1 |
| VEGFR3 | P35916 | Vascular endothelial growth factor receptor 3 |
| FRalpha | P15328 | Folate receptor alphaÊ |
| LDLreceptor | P01130 | Low-density lipoprotein receptor (LDL receptor) |
| EPHB4 | P54760 | Ephrin type-B receptor 4 |
| TFF3 | Q07654 | Trefoil factor 3 |
| CD163 | Q86VB7 | Scavenger receptor cysteine-rich type 1 protein M130Ê |
| GRN | P28799 | Granulins |
| LTBR | P36941 | Tumor necrosis factor receptor superfamily member 3 |
| | | (Lymphotoxin-beta receptor) |
| TLT2 | Q5T2D2 | Trem-like transcript 2 protein (TLT-2) |
| TFPI | P10646 | Tissue factor pathway inhibitor |
| PAI | P05121 | Plasminogen activator inhibitor 1 |
| TR | P02786 | Transferrin receptor protein 1 |
| GDF15 | Q99988 | Growth/differentiation factor 15 (GDF-15) |
| DLK1 | P80370 | Protein delta homolog 1 (DLK-1) |
| vWF | P04275 | von Willebrand factor |
| CCL16 | O15467 | C-C motif chemokine 16 |
| OPG | O00300 | Tumor necrosis factor receptor superfamily member 11B |
| LAPTGFbeta1 | P01137 | Latency-associated peptide transforming growth factor beta-1 |
| | | (LAP TGF-beta-1) |
| uPA | P00749 | Urokinase-type plasminogen activator |
| PDL1 | Q9NZQ7 | Programmed cell death 1 ligand 1 (PD-L1) |
| HGF | P14210 | Hepatocyte growth factor |
| ARTN | Q5T4W7 | Artemin |
| CCL28 | Q9NR13 | C-C motif chemokine 28 |
| NRP2 | O60462 | Neuropilin-2 |
| UNC5C | O95185 | Netrin receptor UNC5C (protein unc-5 homolog C) |
| SMOC2 | Q9H3U7 | SPARC-related modular calcium-binding protein 2Ê |
| EFNA4 | P52798 | EPH-related receptor tyrosine kinase ligand 4 (Ephrin-A4) |
| SCARB2 | Q14108 | Lysosome membrane protein 2 |
| PRTG | Q2VWP7 | Protogenin (Protein Shen-Dan) |
| SMPD1 | P17405 | Sphingomyelin phosphodiesterase |
| MSR1 | P21757 | Macrophage scavenger receptor types I and IIÊ |
| sFRP3 | Q92765 | Secreted frizzled-related protein 3 (sFRP-3) |
| EPHB6 | O15197 | Ephrin type-8 receptor 6 (HEP) |
| SIGLEC1 | Q9BZZ2 | Sialoadhesin (Siglec-1) |
| LAYN | Q6UX15 | Layilin |
| WFIKKN1 | Q96N28 | WAP, Kazal, immunoglobulin, Kunitz and NTR domain-containing |
| | | protein 1 |
| GMCSFRalpha | P15509 | Granulocyte-macrophage colony-stimulating factor receptor |
| | | subunit alpha (GM-CSF-R-alpha) |
| BetaNGF | P01138 | Beta-nerve growth factor (Beta-NGF) |
| CD200 | P41217 | OX-2 membrane glycoprotein (CD antigen CD200) |
| GCSF | P09919 | Granulocyte colony-stimulating factor (G-CSF) |
| PVR | P15151 | Poliovirus receptor (Nectin-like protein 5) |
| TNFRSF12A | Q9NP84 | Tumor necrosis factor receptor superfamily member 12A |
| CXCL16 | Q9H2A7 | C-X-C motif chemokine 16 |
| IL1RT1 | P14778 | Interleukin-1 receptor type 1 (IL-1R-1) (IL-1RT-1) |
| FAS | P25445 | Tumor necrosis factor receptor superfamily member 6 (Apo-1 antigen) |
| PCSK9 | Q8NBP7 | Proprotein convertase subtilisin/kexin type 9 |
| OPN | P10451 | Osteopontin |
| CTSD | P07339 | Cathepsin D |
| Gal4 | P56470 | Galectin-4 (Gal-4) |
| CCL15 | Q16663 | C-C motif chemokine 15 |
| ST2 | Q01638 | Interleukin-1 receptor-like 1 |
| tPA | P00750 | Tissue-type plasminogen activator (t-PA) |
| EGFR | P00533 | Epidermal growth factor receptor |
| IL18BP | O95998 | Interleukin-18-binding protein (IL-18BP) |
| CTSZ | Q9UBR2 | Cathepsin Z |
| TNFR1 | P19438 | Tumor necrosis factor receptor superfamily member 1A |
| | | (Tumor necrosis factor receptor 1) (TNF-R1) |
| SKR3 | P37023 | Serine/threonine-protein kinase receptor R3 (SKR3) |
| CPM | P14384 | Carboxypeptidase M (CPM) |
| PDGFRalpha | P16234 | Platelet-derived growth factor receptor alpha (PDGF-R-alpha) |
| CTSC | Q99895 | Chymotrypsin-C |
| DDR1 | Q08345 | Epithelial discoidin domain-containing receptor 1 |
| CTSS | P25774 | Cathepsin S |
| NCDase | Q9NR71 | Neutral ceramidase (N-CDase) (NCDase) |
| NAAA | Q02083 | N-acylethanolamine-hydrolyzing acid amidase |
| N2DL2 | Q9BZM5 | UL16-binding protein 2 (ALCAN-alpha) |
| PLXNB1 | O43157 | Plexin-B1 |
| TNFRSF21 | O75509 | Tumor necrosis factor receptor superfamily member 21 |
| CLM1 | Q8TDQ1 | CMRF35-like molecule 1 (CLM-1) |
| SPOCK1 | Q08629 | Testican-1 |
| EDA2P | Q9HAV5 | Tumor necrosis factor receptor superfamily member 27 |
| NrCAM | Q92823 | Neuronal cell adhesion molecule (Nr-CAM) |

TABLE 5
Labor-associated proteins that significantly changed expression levels

| Proteins | Uniprot ID | Protein name |
|---|---|---|
| 4EBP1 | Q13541 | Eukaryotic translation initiation factor 4E-binding protein 1 (4E-BP1) |
| | | (eIF4E-binding protein 1) |
| 5′NT | P21589 | 5′-nucleotidase (5′-NT) |
| ABL1 | P00519 | Tyrosine-protein kinase ABL1 |
| ADAM22 | Q9P0K1 | Disintegrin and metalloproteinase domain-containing protein 22 |
| ADAMTS15 | Q8TE58 | A disintegrin and metalloproteinase with thrombospondin motifs 15 |
| ALCAM | Q13740 | CD166 antigen (Activated leukocyte cell adhesion molecule) |
| Alpha2MRAP | P30533 | Alpha-2-macroglobulin receptor-associated protein (Alpha-2-MRAP) |
| AR | P10275 | Androgen receptor (Dihydrotestosterone receptor) |
| ARTN | Q5T4W7 | Artemin |
| BetaNGF | P01138 | Beta-nerve growth factor (Beta-NGF) |
| CASP3 | P42574 | Caspase-3 (CASP-3) |
| CCL15 | Q16663 | C-C motif chemokine 15 |
| CCL16 | O15467 | C-C motif chemokine 16 |
| CCL22 | O00626 | C-C motif chemokine 22 |
| CCL23 | P55773 | C-C motif chemokine 23 |
| CCL24 | O00175 | C-C motif chemokine 24 |
| CD200 | P41217 | OX-2 membrane glycoprotein (CD antigen CD200) |
| CD200R1 | Q8TD46 | Cell surface glycoprotein CD200 receptor 1 |
| CD244 | Q9BZW8 | Natural killer cell receptor 2B4 |
| CD38 | P28907 | ADP-ribosyl cyclase/cyclic ADP-ribose hydrolase 1 |
| CD48 | P09326 | CD48 antigen |
| CD93 | Q9NPY3 | Complement component C1q receptor |
| CDH5 | P33151 | Cadherin-5 |
| CHI3L1 | P36222 | Chitinase-3-like protein 1 |
| CLEC10A | Q8IUN9 | C-type lectin domain family 10 member A |
| CLEC1B | Q9P126 | C-type lectin domain family 1 member B |
| CLM1 | Q8TDQ1 | CMRF35-like molecule 1 (CLM-1) |
| CNTN1 | Q12860 | Contactin-1 (Glycoprotein gp135) |
| COL1A1 | P02452 | Collagen alpha-1(I) chain (Alpha-1 type I collagen) |
| CPE | P16870 | Carboxypeptidase E |
| CPM | P14384 | Carboxypeptidase M (CPM) |
| CRTAM | O95727 | Cytotoxic and regulatory T-cell molecule |
| CSF1 | P09603 | Macrophage colony-stimulating factor 1 (CSF-1) (M-CSF) (MCSF) |
| CSTB | P04080 | Cystatin-B (CPI-B) |
| CTSC | Q99895 | Chymotrypsin-C |
| CTSD | P07339 | Cathepsin D |
| CTSS | P25774 | Cathepsin S |
| CTSV | O60911 | Cathepsin L2 |
| CTSZ | Q9UBR2 | Cathepsin Z |
| CXCL1 | P09341 | Growth-regulated alpha protein (C-X-C motif chemokine 1) |
| CXCL13 | O43927 | C-X-C motif chemokine 13 |
| CXCL16 | Q9H2A7 | C-X-C motif chemokine 16 |
| CXCL5 | P42830 | C-X-C motif chemokine 5 |
| CXCL6 | P80162 | C-X-C motif chemokine 6 |
| CXL17 | Q6UXB2 | C-X-C motif chemokine 17 |
| DDR1 | Q08345 | Epithelial discoidin domain-containing receptor 1 |
| DKN1A | P38936 | Cyclin-dependent kinase inhibitor 1 |
| DLK1 | P80370 | Protein delta homolog 1 (DLK-1) |
| DLL1 | O00548 | Delta-like protein 1 |
| DNER | Q8NFT8 | Delta and Notch-like epidermal growth factor-related receptor |
| DRAXIN | Q8NBI3 | Draxin |
| EDA2R | Q9HAV5 | Tumor necrosis factor receptor superfamily member 27 |
| EGF | P01133 | Pro-epidermal growth factor (EGF) |
| EGFR | P00533 | Epidermal growth factor receptor |
| EpCAM | P16422 | Epithelial cell adhesion molecule (Ep-CAM) |
| EPHB4 | P54760 | Ephrin type-B receptor 4 |
| EPHB6 | O15197 | Ephrin type-B receptor 6 (HEP) |
| ERBB2 | P04626 | Receptor tyrosine-protein kinase erbB-2 |
| ERBB3 | P21860 | Receptor tyrosine-protein kinase erbB-3 |
| ERBB4 | Q15303 | Receptor tyrosine-protein kinase erbB-4 |
| ESM1 | Q9NQ30 | Endothelial cell-specific molecule 1 (ESM-1) |
| FADD | Q13158 | FAS-associated death domain protein |
| FAS | P25445 | Tumor necrosis factor receptor superfamily member 6 (Apo-1 antigen) |
| FCRLB | Q6BAA4 | Fc receptor-like B |
| FGFBP1 | Q14512 | Fibroblast growth factor-binding protein 1 |
| FRalpha | P15328 | Folate receptor alpha (FR-alpha) |
| FURIN | P09958 | Furin |
| Gal1 | P09382 | Galectin-1 (Gal-1) |
| Gal4 | P56470 | Galectin-4 (Gal-4) |
| gal8 | O00214 | Galectin-8 (Gal-8) |
| GCP5 | P78333 | Glypican-5 |
| GCSF | P09919 | Granulocyte colony-stimulating factor (G-CSF) |
| GDF15 | Q99988 | Growth/differentiation factor 15 (GDF-15) |
| GDF8 | O14793 | Growth/differentiation factor 8 (GDF-8) |
| GDNFRalpha3 | O60609 | GDNF family receptor alpha-3 (GDNF receptor alpha-3) |
| GFRalpha1 | P56159 | GDNF family receptor alpha-1 (GDNF receptor alpha-1) |
| GMCSFRalpha | P15509 | Granulocyte-macrophage colony-stimulating factor receptor subunit alpha |
| | | (GM-CSF-R-alpha) |
| GPNMB | Q14956 | Transmembrane glycoprotein NMB |
| GRN | P28799 | Granulins |
| GZMA | P12544 | Granzyme A |
| HAGH | Q16775 | Hydroxyacylglutathione hydrolase |
| hK11 | Q9UBX7 | Kallikrein-11 (hK11) |
| hK14 | Q9P0G3 | Kallikrein-14 (hK14) |
| ICAM2 | P13598 | Intercellular adhesion molecule 2 (ICAM-2) |
| ICOSLG | O75144 | ICOS ligand (B7 homolog 2) |
| IFNgammaR1 | P15260 | Interferon gamma receptor 1 (IFN-gamma receptor 1) |
| IGF1R | P08069 | Insulin-like growth factor 1 receptor |
| IGFBP1 | P08833 | Insulin-like growth factor-binding protein 1 (IBP-1) |
| IGFBP2 | P18065 | Insulin-like growth factor-binding protein 2 (IBP-2) |
| IGFBP7 | Q16270 | Insulin-like growth factor-binding protein 7 (IBP-7) |
| IL10RB | Q08334 | Interleukin-10 receptor subunit beta (IL-10 receptor subunit beta) |
| IL12B.IL12A | P29459 | Interleukin-12 subunit alpha (IL-12A) |
| IL18BP | O95998 | Interleukin-18-binding protein (IL-18BP) |
| IL1RT1 | P14778 | Interleukin-1 receptor type 1 (IL-1R-1) (IL-1RT-1) |
| IL1RT2 | P27930 | Interleukin-1 receptor type 2 (IL-1R-2) (IL-1RT-2) |
| IL8 | P10145 | Interleukin-8 (IL-8) |
| ITGB5 | P18084 | Integrin beta-5 |
| JAMA | Q9Y624 | Junctional adhesion molecule A (JAM-A) |
| KYNU | Q16719 | Kynureninase |
| LAIR2 | Q6ISS4 | Leukocyte-associated immunoglobulin-like receptor 2 (LAIR-2) |
| LAT | O43561 | Linker for activation of T-cells family member 1 |
| LAYN | Q6UX15 | Layilin |
| LDLreceptor | P01130 | Low-density lipoprotein receptor (LDL receptor) |
| LIFR | P42702 | Leukemia inhibitory factor receptor (LIF receptor) |
| LTBR | P36941 | Tumor necrosis factor receptor superfamily member 3 |
| | | (Lymphotoxin-beta receptor) |
| LXN | Q9BS40 | Latexin |
| LY9 | Q9HBG7 | T-lymphocyte surface antigen Ly-9 (Cell surface molecule Ly-9) |
| LYN | P07948 | Tyrosine-protein kinase Lyn |
| LYPD3 | O95274 | Ly6/PLAUR domain-containing protein 3 |
| MADhomolog5 | Q99717 | Mothers against decapentaplegic homolog 5 (MAD homolog 5 |
| MANF | P55145 | Mesencephalic astrocyte-derived neurotrophic factor |
| MDGA1 | Q8NFP4 | MAM domain-containing glycosylphosphatidylinositol anchor protein 1 |
| MEPE | Q9NQ76 | Matrix extracellular phosphoglycoprotein |
| MetAP2 | P50579 | Methionine aminopeptidase 2 |
| MK | P21741 | Midkine (MK) (Amphiregulin-associated protein) (ARAP) |
| MMP10 | P09238 | Stromelysin-2 (SL-2) |
| MMP3 | P08254 | Stromelysin-1 (SL-1) |
| MMP9 | P14780 | Matrix metalloproteinase-9 (MMP-9) |
| MPO | P05164 | Myeloperoxidase (MPO) |
| N2DL2 | Q9BZM5 | UL16-binding protein 2 (ALCAN-alpha) |
| NAAA | Q02083 | N-acylethanolamine-hydrolyzing acid amidase |
| NCDase | Q9NR71 | Neutral ceramidase (N-CDase) (NCDase) |
| NrCAM | Q92823 | Neuronal cell adhesion molecule (Nr-CAM) |
| NRP2 | O60462 | Neuropilin-2 |
| NTRK2 | Q16620 | BDNF/NT-3 growth factors receptor |
| NTRK3 | Q16288 | NT-3 growth factor receptor |
| OPG | O00300 | Tumor necrosis factor receptor superfamily member 11B |
| PAI | P05121 | Plasminogen activator inhibitor 1 |
| PCSK9 | Q8NBP7 | Proprotein convertase subtilisin/kexin type 9 |
| PDGFRalpha | P16234 | Platelet-derived growth factor receptor alpha (PDGF-R-alpha) |
| PDGFsubunitA | P04085 | Platelet-derived growth factor subunit A (PDGF subunit A) (PDGF-1) |
| PDL1 | Q9NZQ7 | Programmed cell death 1 ligand 1 (PD-L1) |
| PECAM1 | P16284 | Platelet endothelial cell adhesion molecule (PECAM-1) |
| PLC | P98160 | Basement membrane-specific heparan sulfate proteoglycan core protein |
| | | (Perlecan) |
| PLXNB1 | O43157 | Plexin-B1 |
| PLXNB3 | Q9ULL4 | Plexin-B3 |
| PON3 | Q15166 | Serum paraoxonase/lactonase 3 |
| PPY | P01298 | Pancreatic prohormone (Pancreatic polypeptide) |
| PRTG | Q2VWP7 | Protogenin (Protein Shen-Dan) |
| PVR | P15151 | Poliovirus receptor (Nectin-like protein 5) |
| PVRL4 | Q96NY8 | Nectin-4 |
| RET | P07949 | Proto-oncogene tyrosine-protein kinase receptor Ret |
| RETN | Q9HD89 | Resistin |
| ROBO2 | Q9HCK4 | Roundabout homolog 2 |
| RSPO1 | Q2MKA7 | R-spondin-1 |
| RSPO3 | Q9BXY4 | R-spondin-3 |
| S100A11 | P31949 | Protein S100-A11 |
| S100A4 | P26447 | Protein S100-A4 |
| SCAMP3 | O14828 | Secretory carrier-associated membrane protein 3 |
| SCARB2 | Q14108 | Lysosome membrane protein 2 |
| SCARF2 | Q96GP6 | Scavenger receptor class F member 2 |
| SCGB3A2 | Q96PL1 | Secretoglobin family 3A member 2 |
| SELP | P16109 | P-selectin |
| SEZ6L | Q9BYH1 | Seizure 6-like protein |
| sFRP3 | Q92765 | Secreted frizzled-related protein 3 (sFRP-3) |
| SIGLEC1 | Q9BZZ2 | Sialoadhesin (Siglec-1) |
| Siglec9 | Q9Y336 | Sialic acid-binding Ig-like lectin 9 (Siglec-9) |
| SKR3 | P37023 | Serine/threonine-protein kinase receptor R3 (SKR3) |
| SLAMF1 | Q13291 | Signaling lymphocytic activation molecule |
| SMPD1 | P17405 | Sphingomyelin phosphodiesterase |
| SPARC | P09486 | SPARC (Secreted protein acidic and rich in cysteine) |
| SPOCK1 | Q08629 | Testican-1 |
| SPON1 | Q9HCB6 | Spondin-1 (F-spondin) |
| ST2 | Q01638 | Interleukin-1 receptor-like 1 |
| STAMPB | O95630 | STAM-binding protein |
| SYND1 | P18827 | Syndecan-1 |
| TCL1A | P56279 | T-cell leukemia/lymphoma protein 1A |
| TFF3 | Q07654 | Trefoil factor 3 |
| TFPI2 | P48307 | Tissue factor pathway inhibitor 2 (TFPI-2) |
| TGFalpha | P01135 | Protransforming growth factor alpha |
| THY1 | P04216 | Thy-1 membrane glycoprotein |
| TLR3 | O15455 | Toll-like receptor 3 |
| TLT2 | Q5T2D2 | Trem-like transcript 2 protein (TLT-2) |
| TMPRSS5 | Q9H3S3 | Transmembrane protease serine 5 |
| TNFR1 | P19438 | Tumor necrosis factor receptor superfamily member 1A |
| | | (Tumor necrosis factor receptor 1) (TNF-R1) |
| TNFR2 | P20333 | Tumor necrosis factor receptor superfamily member 1B |
| | | (Tumor necrosis factor receptor 2) (TNF-R2) |
| TNFRSF10C | O14798 | Tumor necrosis factor receptor superfamily member 10C |
| TNFRSF12A | Q9NP84 | Tumor necrosis factor receptor superfamily member 12A |
| TNFRSF4 | P43489 | Tumor necrosis factor receptor superfamily member 4 |
| TNFSF10 | P50591 | Tumor necrosis factor ligand superfamily member 10 |
| TNFSF13 | O75888 | Tumor necrosis factor ligand superfamily member 13 |
| TNFSF13B | Q9Y275 | Tumor necrosis factor ligand superfamily member 13B |
| tPA | P00750 | Tissue-type plasminogen activator (t-PA) |
| TRAIL | P50591 | TNF-related apoptosis-inducing ligand |
| TRANCE | O14788 | TNF-related activation-induced cytokine |
| TRAP | P13686 | Tartrate-resistant acid phosphatase type 5 |
| TWEAK | O43508 | Tumor necrosis factor (Ligand) superfamily, member 12 |
| UPAR | Q03405 | Urokinase plasminogen activator surface receptor |
| VEGFR2 | P35968 | Vascular endothelial growth factor receptor 2 |
| VEGFR3 | P35916 | Vascular endothelial growth factor receptor 3 |
| VIM | P08670 | Vimentin |
| vWF | P04275 | von Willebrand factor |
| WFDC2 | Q14508 | WAP four-disulfide core domain protein 2 |
| WIF1 | Q9Y5W5 | Wnt inhibitory factor 1 |
| WISP1 | O95388 | WNT1-inducible-signaling pathway protein 1 |
| ACE2 | Q9BYF1 | Angiotensin-converting enzyme 2¾ |
| ADM | P35318 | Adrenomedullin¾ |
| ANG-1 | Q15389 | Angiopoietin-1 |
| BMP-6 | P22004 | Bone morphogenetic protein 6 |
| CCL17 | Q92583 | C-C motif chemokine 17 |
| CD4 | P01730 | T-cell surface glycoprotein CD4 |
| CD40-L | P29965 | CD40 ligand |
| CD84 | Q9UIB8 | SLAM family member 5 |
| CTRC | Q99895 | Chymotrypsin C |
| CTSL1 | P07711 | Cathepsin L1 |
| CXCL1 | P09341 | C-X-C motif chemokine 1 |
| DECR1 | Q16698 | 2,4-dienoyl-CoA reductase |
| FS | P19883 | Follistatin |
| GDF-2 | Q9UK05 | Growth/differentiation factor 2 |
| GLO1 | Q04760 | Lactoylglutathione lyase |
| GT | P51161 | Gastrotropin |
| HB-EGF | Q99075 | Proheparin-binding EGF-like growth factor |
| HO-1 | P09601 | Heme oxygenase 1 |
| IDUA | P35475 | Alpha-L-iduronidase |
| IL-17D | Q8TAD2 | Interleukin -17D |
| IL-1ra | P18510 | Interleukin -1 receptor antagonist protein |
| IL-4RA | P24394 | Interleukin -4 receptor subunit alpha |
| IL1RL2 | Q9HB29 | Interleukin-1 receptor-like 2 |
| IL27 | Q14213 | Interleukin -27 |
| ITGB1BP2 | Q9UKP3 | Melusin |
| LEP | P41159 | leptin |
| LOX-1 | P78380 | Lectin-like oxidized LDL receptor 1¾ |
| LPL | P06858 | Lipoprotein lipase |
| MMP-12 | P39900 | Matrix metalloproteinase -12 |
| NEMO | Q9Y6K9 | NF-kappa-B essential modulator¾ |
| PAPPA | Q13219 | Pappalysin-1 |
| PAR-1 | P25116 | Proteinase-activated receptor 1 |
| PD-L2 | Q9BQ51 | Programmed cell death 1 ligand 2¾ |
| PDGF subunit B | P01127 | Platelet-derived growth factor subunit B |
| PIGF | P49763 | Placenta growth factor |
| PRSS8 | Q16651 | Prostasin |
| RAGE | Q15109 | Receptor for advanced glycosylation end products |
| REN | P00797 | Renin |
| SOD2 | P04179 | Superoxide dismutase [Mn], mitochondrial (SOD2) |
| SPON2 | Q9BUD6 | Spondin-2 |
| STK4 | Q13043 | Serine/threonine-protein kinase 4 |
| TF | P13726 | Tissue factor |
| THBS2 | P35442 | Thrombospondin-2 |
| TIE2 | Q02763 | Angiopoietin-1 receptor |
| TM | P07204 | Thrombomodulin |
| TNFRSF10A | O002200 | Tumor necrosis factor receptor superfamily member 10A¾ |
| TNFRSF11A | Q9Y6Q6 | Tumor necrosis factor receptor superfamily member 11A¾ |
| TRAIL-R2 | O14763 | TNF-related apoptosis-inducing ligand receptor 2 |
| VEGF-D | O43915 | Vascular endothelial growth factor D¾ |

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

1.-40. (canceled)

41. A method for determining that a pregnant subject has or is at an elevated risk of having a pregnancy complication, comprising:
(a) obtaining a biological sample of the pregnant subject;
(b) assaying the biological sample of the pregnant subject for at least two members selected from the group consisting of a protein biomarker, a metabolite biomarker, and a lipid biomarker, to yield biomarker data;
(c) computer processing the biomarker data generated in (b); and
(d) determining that the pregnant subject has or is at the elevated risk of having the pregnancy complication, based at least in part on the computer processing in (c).

42. The method of claim 41, wherein (b) comprises assaying the biological sample of the pregnant subject for the protein biomarker and the metabolite biomarker.

43. The method of claim 41, wherein (b) comprises assaying the biological sample of the pregnant subject for the protein biomarker and the lipid biomarker.

44. The method of claim 41, wherein (b) comprises assaying the biological sample of the pregnant subject for the metabolite biomarker and the lipid biomarker.

45. The method of claim 41, wherein (b) comprises assaying metabolites of the biological sample.

46. The method of claim 45, wherein the metabolites comprise a sugar, an amino acid, a nucleotide, an antioxidant, an organic acid, a polyol, or a vitamin.

47. The method of claim 45, wherein the metabolites comprise a member selected from the group listed in Table 2.

48. The method of claim 41, wherein (b) comprises assaying proteins of the biological sample.

49. The method of claim 48, wherein the proteins are assayed using an immunoassay.

50. The method of claim 48, wherein the proteins are assayed using a multiplex proximity extension assay (PEA) or a microsphere-based multiplex assay.

51. The method of claim 48, wherein the proteins comprise one or more members selected from the group consisting of: NTRK2, LAIR2, CD200R1, LXN, DRAXIN, R0B02, CD93, NTRK3, MDGA1, CRTAM, IL12B/IL12A, RGMA, IL2RA, ESM1, FcRL2, UPAR, MCP2, IL5Ralpha, CLM1, uPA, CCL28, PCSK9, PDGFRalpha, SMPD1, SKR3, DLK1, NRP2, MSR1, GMCSFRalpha, CTSC, RET, SMOC2, PRTG, PVRL4, ST2, NrCAM, SYND1, TNFRSF12A, DDR1, CD200, GRN, and PAI1.

52. The method of claim 41, wherein the computer processing in (c) comprises applying a computational model to the biomarker data generated in (b).

53. The method of claim 52, wherein the computational model comprises a regression model.

54. The method of claim 41, wherein (d) further comprises performing a clinical assessment on the pregnant subject, wherein the clinical assessment is selected from the group consisting of: a medical imaging, a fetal monitoring, a chorionic villus sampling, an amniocentesis, an evaluation for preeclampsia, an evaluation for gestational hypertension, an evaluation for gestational diabetes, an evaluation for preterm labor, an evaluation for signs of preterm rupture of membranes, and a glucose screening.

55. The method of claim 41, wherein (d) comprises determining a gestational progress or a gestational health of a fetus of the pregnant subject based at least in part on the computer processing in (c), and determining that the pregnant subject has or is at the elevated risk of having the pregnancy complication based at least in part on the gestational progress or the gestational health of the fetus.

56. The method of claim 55, wherein the gestational progress or the gestational health is selected from the group consisting of: a gestational age of the fetus, a time to delivery, a labor onset, and any combination thereof.

57. The method of claim 56, wherein the gestational progress or the gestational health comprises the gestational age of the fetus.

58. The method of claim 56, wherein the gestational progress or gestational health comprises the time to delivery.

59. The method of claim 55, wherein the pregnancy complication is selected from the group consisting of: early maladaptive pregnancy, spontaneous abortion, gestational diabetes, gestational hypertension, gestational trophoblastic disease, preeclampsia, hyperemesis gravidarum, pre-term labor, post-term pregnancy, post-term labor, and any combination thereof.

60. The method of claim 59, wherein the pregnancy complication comprises the pre-term labor.

61. The method of claim 59, wherein the gestational complication comprises the preeclampsia.

62. The method of claim 41, further comprising computer processing biomarker data generated from biological samples obtained or derived from the pregnant subject at a plurality of different time points; and comparing the computer processed biomarker data to each other to determine that the pregnant subject has or is at the elevated risk of having the pregnancy complication.

63. The method of claim 62, wherein the plurality of different time points comprise a member selected from the group consisting of: first missed menstruation, fertilization, birth, first trimester, second trimester, third trimester, 4 weeks gestation, 6 weeks gestation, 8 weeks gestation, 10 weeks gestation, 12 weeks gestation, 16 weeks gestation, 24 weeks gestation, 28 weeks gestation, 32 weeks gestation, 36 weeks gestation, 40 weeks gestation, 1 week before delivery, 2 weeks before delivery, 3 weeks before delivery, 4 weeks before delivery, 6 weeks before delivery, and 8 weeks before delivery.

64. The method of claim 41, further comprising administering a treatment to the pregnant subject for the pregnancy complication based on the determining in (d).

65. The method of claim 64, wherein the treatment is selected from the group consisting of: a medication, an intravenous fluid, an antibiotic, a cervical cerclage, folic acid, iron, calcium, vitamin D, docosahexaenoic acid (DHA), iodine, a dietary supplement, estrogen, progestogen, progesterone, dydrogesterone, an induction of labor, a delivery of the fetus, a Caesarian delivery of the fetus, and a surgical procedure.

66. The method of claim 65, wherein the medication comprises a tocolytic medication.

67. The method of claim 66, wherein the tocolytic medication is selected from the group consisting of: indomethacin, magnesium sulfate, orciprenaline, ritodrine, terbutaline, salbutamol, nifedipine, fenoterol, nylidrin, isoxsuprine, hexoprenaline, and atosiban.

68. The method of claim 41, wherein the biological sample is selected from the group consisting of: a blood sample, a plasma sample, a stool sample, a urine sample, a saliva sample, and a biopsy sample.

69. The method of claim 68, wherein the biological sample is the plasma sample.

70. A method for determining that a pregnant subject has or is at an elevated risk of having a pregnancy complication, comprising:
(a) obtaining a biological sample of the pregnant subject;
(b) assaying the biological sample of the pregnant subject for at least one protein biomarker to yield biomarker data based on a presence of the at least one protein biomarker in the biological sample, wherein the at least one protein is assayed using a multiplex proximity extension assay (PEA) or a microsphere-based multiplex assay;
(c) computer processing the biomarker data generated in (b); and
(d) determining that the pregnant subject has or is at the elevated risk of having the pregnancy complication, based at least in part on the computer processing in (c).
